(19) United States
(12) Patent Application Publication
(10) Pub. No.: US 2019/0070178 A1
(43) Pub. Date: Mar. 7, 2019

(54) METHODS FOR TREATING OR PREVENTING CARDIOVASCULAR DISORDERS AND LOWERING RISK OF CARDIOVASCULAR EVENTS

(71) Applicants: DALCOR PHARMA UK LTD., STOCKPORT ZUG BRANCH, Zug (CH); MONTREAL HEART INSTITUTE, Montreal (CA)

(72) Inventors: Marie-Pierre DUBÉ, Montreal (CA); Jean-Claude TARDIF, Montreal (CA); Eric RHÉAUME, Montreal (CA)

(21) Appl. No.: 16/115,135

(22) Filed: Aug. 28, 2018

Related U.S. Application Data
(60) Provisional application No. 62/558,137, filed on Sep. 13, 2017.
(60) Provisional application No. 62/551,692, filed on Aug. 29, 2017.

(51) Int. Cl. A61K 31/517; A61P 9/10; A61K 31/265; A61P 3/06; A61P 9/14; A61P 3/10; C12Q 1/6827; A61K 31/167; A61K 31/122

(52) U.S. Cl. CPC A61K 31/517; A61P 9/10; A61K 31/265; A61P 3/06; A61P 9/14; A61K 31/4245; C12Q 1/6827; A61K 31/167; A61K 31/122; A61K 2300/00; A61P 3/10

(57) ABSTRACT

The invention provides compositions and methods useful for treating or preventing cardiovascular disorders and lowering risk of cardiovascular events.

### CROSS-REFERENCE TO RELATED APPLICATIONS

This application claims the benefit of U.S. provisional application No. 62/558,137, filed Sep. 13, 2017, and U.S. provisional application No. 62/551,692, filed Aug. 29, 2017, the disclosure of each of which is incorporated by reference herein in its entirety.

### FIELD OF THE INVENTION

The present disclosure provides compositions and methods useful for treating or preventing cardiovascular disorders and lowering risk of cardiovascular events.

### BACKGROUND

Lowing low-density lipoproteins (LDL) is an important therapeutic strategy in the management of cardiovascular disease. Indeed statin drugs, which lower LDL, such as Crestor, Lipitor, Pravachol, and Zocar are widely used and among the most prescribed drugs. For some time it has also been generally accepted that increasing High-density lipoproteins (HDL) could also be therapeutic in cardiovascular disease. Several HDL-raising drugs have been developed including: niacin and CETP inhibitors such as torcetrapib, anacetrapib, evacetrapib and dalcetrapib.

Cholesteryl ester transfer protein (CETP) also called plasma lipid transfer protein is a hydrophobic glycoprotein that is synthesized in several tissues but mainly in the liver. CETP promotes bidirectional transfer of cholesteryl esters and triglyceride between all plasma lipoprotein particles. The first evidence of the effect of CETP activity on plasma lipoproteins was provided by observations in people with genetic deficiencies of CETP. The first CETP mutation was identified in Japan in 1989 as a cause of markedly elevated HDL-C. Ten mutations associated with CETP deficiency have since been identified in Asians and one in Caucasians. It was found in Japan that 57% of subjects with levels of HDL-C>100 mg/dL have mutations of the CETP gene. In addition, 37% of Japanese with levels HDL-C between 75-100 mg/dL have mutations of the CETP gene. Subsequently, studies of animals treated with an anti-CETP antibody showed that CETP inhibition resulted in a substantial increase in the concentration of HDL-C. Consistent with these observations in CETP deficient patients and rabbits treated with an anti-CETP antibody, it has since been found that treatment of humans with CETP inhibitor drugs increases the concentration of HDL cholesterol and apoA-I (the major apolipoprotein in HDLs). Numerous epidemiologic studies have correlated the effects of variations in CETP activity with coronary heart disease risk including studies of human mutations (Hirano et al., Curr. Opin. Lipido. (2000) 11(4), 389-396).

Atherosclerosis and its clinical consequences, including coronary heart disease (CHD), stroke and peripheral vascular disease represents an enormous burden on health care systems internationally. CETP inhibitors have been under development for some time with the expectation that they will be useful for treating or preventing atherosclerosis. A number of classes of CETP inhibitor drugs have been shown to increase HDL, decrease LDL in humans and to have therapeutic effects for treating atherosclerosis and cardiovascular disease including dalcetrapib, torcetrapib, anacetrapib, evacetrapib, BAY 60-5521 and others. However, there is evidence that these drugs may not be safe and effective in all patients. The clinical trial for torcetrapib was terminated in Phase III due to incidence of mortality in patient to whom torcetrapib and atorvastatm were administered concomitantly compared to patients treated with atorvastatin alone. The clinical trial for dalcetrapib was also halted in Phase III in this case due to a lack of efficacy relative to statins alone.

The dalcetrapib mortality and morbidity trial (dal-OUTCOMES) was a double-blind, randomized, placebo-controlled, parallel group, multi-centre study in stable CHD patients recently hospitalized for acute coronary syndrome (ACS). The study was conducted to test the hypothesis that CETP inhibition will reduce the risk of recurrent cardiovascular events in patients with recent ACS by raising levels of HDL-C through CETP inhibition. Eligible patients entered a single-blind placebo run-in period of approximately 4 to 6 weeks to allow for patients to stabilize and for completion of planned revascularization procedures. At the end of the run-in period, eligible patients in stable condition were randomized in a 1:1 ratio to 600 mg of dalcetrapib or placebo on top of evidence-based medical care for ACS. The dal-OUTCOMES study resulted in unexpected observations related to cardiovascular disease progression. Despite a marked increase in HDL-c, patients on treatment did not show a significant reduction in cardiovascular events and the study was terminated.

A pharmacogenomic study of the dal-OUTCOMES study population was conducted to study the inter-individual variation in dalcetrapib response and to identify genetic markers for predicting therapeutic response to dalcetrapib, or other CETP inhibitors, for patient stratification and for treatment selection. It was discovered that the effects of dalcetrapib on atherosclerotic outcomes are determined by correlated polymorphisms in the Adenylate Cyclase Type 9 (ADCY9) gene. Specifically, 20 SNPs that occur in the ADCY9 gene on chromosome 16, including rs11647778, rs1967309, rs12595857, rs2239310, rs11647828, rs8049452, rs12935810, rs74702385, rs17136707, rs8061182, rs111590482, rs4786454, rs2283497, rs2531967, rs3730119, rs2531971, rs2238448, rs11599911, rs12920508, and rs13337675 were found to be associated with response to a CETP inhibitor, dalcetrapib. In particular rs11647778 or rs1967309 are both in strong linkage disequilibrium together (r2=0.79) and strongly associated with response to dalcetrapib. This finding was disclosed in WO2014154606A1, WO2016016157A1, Tardif et al., Circulation: Cardiovascular Genetics, (2015) 8:372-382, and Tardif et al., Circulation: Cardiovascular Genetics, (2016) 9:340-348, each of which is incorporated by reference herein.

Adenylate cyclases (ADCYs) catalyze the formation of cyclic adenosine-3′,5′-monophosphate (cAMP), the universal second messenger, from adenosine triphosphate (ATP). The adenylate cyclase family consists of 10 members (ADCY1-ADCY10) with high primary sequence similarity at the catalytic site. ADCY9 is the ninth, least well characterized and widely distributed isoform of adenylate cyclase. Polymorphisms in the ADCY9 gene have been shown to affect individual response to inhaled therapy in patients with asthma. (Kim et al., J Clin Pharm Ther. (2011) 36:399-405) Recently, another ADCY9 gene polymorphism was shown to be associated with bodymass index and clinical classes of obesity. (Berndt et al., Nat Genet. (2013) 45:501-512) However, the underlying mechanisms responsible for the interaction between ADCY9 and CETP has not yet been previously disclosed. There is still a need to develop novel therapeutic compositions and methods for treatment or prophylaxis of subject with cardiovascular disorder.

### SUMMARY OF THE INVENTION

One aspect of the invention provides methods for treating or preventing a cardiovascular disorder, comprising administering to a subject in need thereof an effective amount of: a) a CETP inhibitor; and b) an ADCY inhibitor.

Another aspect of the invention provides methods for reducing risk of a cardiovascular event, comprising administering to a subject in need thereof an effective amount of: a) a CETP inhibitor; and b) an ADCY inhibitor.

Another aspect of the invention provides compositions comprising a) an effective amount of a CETP inhibitor and an ADCY inhibitor; and b) a pharmaceutically acceptable carrier or vehicle.

Another aspect of the invention provides methods for treating or preventing a cardiovascular disorder, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor.

Another aspect of the invention provides methods for reducing the risk of a cardiovascular event, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor.

### BRIEF DESCRIPTION OF THE FIGURES

FIG. 1A shows that Adcy9 mRNA expression (analyzed by RT-qPCR) in the heart was decreased by 50% in Adcy9^{WT/Gt} mice and by at least 90% in Adcy9^{Gt/Gt} mice compared to WT (wild type). FIG. 1E shows that ADCY9 protein expression was abolished in Adcy9^{Gt/Gt} mice compared to WT in the skeletal muscle.

FIG. 1B shows the plasma PCSK9 concentration of WT and Adcy9^{Gt/Gt} mice infected with AAV8-Pcsk9^{D377Y} at baseline, one week after infection and before the start of the atherogenic diet, after 8 weeks and 12 weeks of atherogenic diet.

FIG. 1C shows that PCSK9^{D377Y} induced a reduction of 90% in hepatic LDL receptor expression that was similar in WT and Adcy9^{Gt/Gt}.

FIG. 1D shows the plasma cholesterol levels of WT and Adcy9^{Gt/Gt} mice infected with AAV8-Pcsk9^{D377Y} at baseline, one week after infection and before the start of the atherogenic diet, after 4 weeks, 8 weeks and 12 weeks of atherogenic diet.

FIG. 2A and FIG. 2B show the lipoprotein profile by chromatography of WT (FIG. 2A) and Adcy9^{Gt/Gt} (FIG. 2B) mice at baseline and after 16 weeks of atherogenic diet.

FIG. 3A shows that 11.3±2.1% of the surface of the intima covered with atherosclerotic lesions in WT mice compared to 3.8±0.6% in Adcy9^{Gt/Gt} mice at sacrifice. Atherosclerosis lesions were quantified on whole aorta dissected en face. FIG. 3D shows atherosclerotic lesions in the aorta of WT and Adcy9^{Gt/Gt} mice as revealed by Oil Red 0 staining.

FIG. 3B shows that the cross-sectional quantification of plaque area all along the aortic root of WT and Adcy9^{Gt/Gt} mice. FIG. 3E shows the atherosclerotic lesions in cross-sections of the aortic valve of WT and Adcy9^{Gt/Gt} mice.

FIG. 3C and FIG. 3F show that all WT animals had plaques in the brachiocephalic arteries in contrast to Adcy9^{Gt/Gt} mice (100%, versus 50%, P<0.05). The plaques in WT mice were also larger and presented more frequently fibrin deposits at their surface (P<0.05) and cap ruptures compared to Adcy9^{Gt/Gt}mice.

FIG. 4A shows that CD68-positive foam cells, a key constituent of atherosclerotic plaques, represented 19.0±1.9% and 11.4±2.1% of the lesion area in WT and Adcy9^{Gt/Gt} mice respectively (P<0.05). FIG. 4D shows immune-fluorescent detection of CD68-positive macrophages (foam cells).

FIG. 4B shows in situ hybridization analysis of Adcy9 expression in atherosclerotic lesions at sites of foam cell accumulation in WT and Adcy9^{Gt/Gt} mice.

FIG. 4C and FIG. 4E show immunofluorescence detection of Ki67 which revealed that proliferation of CD68-positive foam cells was significantly decreased in Adcy9^{Gt/Gt} compared to WT (P<0.05).

FIG. 5A shows that endothelial-dependent vasodilatation to ACh in untreated animals (no AAV8-Pcsk9^{D377Y} and no atherogenic diet) was potentiated in the femoral arteries of Adcy9^{Gt/Gt} mice compared to WT (P<0.01); in contrast, endothelial-independent vasodilation to the nitric oxide donor SNP was similar in both groups (FIG. 5E).

FIG. 5B shows that Adcy9 inactivation also potentiated endothelial-dependent vasodilatation in response to increased shear stress (15 dynes/cm²) compared to WT (42.3±7.1% versus 28.2±6.3%, P=0.08).

FIG. 5C and FIG. 5F show that in femoral arteries from atherosclerotic animals (AAV8-Pcsk9^{D377Y} and atherogenic diet) both endothelial-dependent (Ach) and endothelial-independent vasodilatation (SNP) were increased in Adcy9^{Gt/Gt} mice compared to WT mice (P<0.05).

FIG. 5D and FIG. 5G show that the relaxant dose response to ACh (FIG. 5D) and SNP (FIG. 5G) of aorta of animals treated with the AAV8-Pcsk9^{D377Y} and atherogenic diet when comparing Adcy9^{Gt/Gt} to WT mice.

FIG. 6A and FIG. 6D show that the nitric oxide synthase blocker L-NNA inhibited significantly ACh-induced vasodilatation both in WT and Adcy9^{Gt/Gt} mice (P<0.01), but flow-mediated vasodilatation was numerically reduced only in Adcy9^{Gt/Gt} mice.

FIG. 6B and FIG. 6E show that the cyclooxygenase blocker meclofenamate or the cocktail of endothelial-dependent hyperpolarization blockers (TRAM-34 and apamin), inhibited ACh-induced endothelial-dependent vasodilatation in Adcy9^{Gt/Gt} mice (P<0.01 for both pathways) but not in WT mice.

FIG. 6C shows the specificity of the Adcy9 mRNA signal by in situ hybridization in the femoral artery wall was confirmed by absence of dots with the negative control probe.

FIG. 7A shows that Adcy9^{Gt/Gt} mice gained more weight than WT animals during the 16 weeks of atherogenic diet, to reach weights of 45.1±2.4 g and 33.5±1.2 g respectively (P<0.01).

FIG. 7B shows that MM performed at week 14 showed that Adcy9^{Gt/Gt} mice presented more adipose tissue than WT mice.

FIG. 7C shows a doubling of total body adipose tissue volume in Adcy9^{Gt/Gt} mice (9.4±1.2 cm³) compared to WT (4.3±0.4 cm³, P<0.01).

FIG. 7D shows larger inguinal, perirenal, epididymal and interscapular fat depots in Adcy9^{Gt/Gt} mice compared to WT.

FIG. 7E and FIG. 7F show histological sections showing that Adcy9 inactivation resulted in hypertrophic adipocytes in epididymal white adipose tissue (FIG. 7E) and larger lipid droplets in interscapular brown adipose tissue (FIG. 7F).

FIG. 8A shows that with the atherogenic diet, Adcy9Gt/Gt mice showed a significant increase in feed efficiency (1.2±0.1 g/100 kCal) compared to WT (0.6±0.1 g/100 kCal, P<0.01).

FIG. 8B shows RR interval (representing heart rate variability by telemetry) was increased in Adcy9Gt/Gt mice (135±5 ms) compared to WT mice (123±4 ms, P=0.05).

FIG. 8C shows that nocturnal heart rate was lower in Adcy9Gt/Gt mice (447±10 bpm) compared to WT mice (475±10 bpm, P=0.06). pNN(6), which represents the percentage of RR intervals exceeding preceding ones by 6 ms or more and reflects parasympathetic nervous system activity 17, was increased during the night period in Adcy9Gt/Gt mice (21.6±2.4%) compared to WT (14.5±2.2%, P<0.05).

FIG. 9A and FIG. 9C show that CETP^{WT} and CETP^{Gt} mice developed similar atherosclerotic lesions that covered 11.8±1.8% and 8.6±1.9% of the intima area, respectively (P=NS).

FIG. 9B shows percentage of lesion area in the whole aorta in WT, Adcy9^{Gt/Gt} CETP^{WT} and CETP^{Gt} mice.

FIG. 10A shows vasodilatation to ACh in femoral arteries from atherosclerotic CETP^{WT} and CETP^{Gt} mice.

FIG. 10B shows vasodilatation to SNP in femoral arteries from atherosclerotic CETP^{WT} and CETP^{Gt} mice.

FIG. 11A shows progression of body weight upon atherogenic treatment in WT, Adcy9^{Gt/Gt}, CETP^{WT} and CETP^{Gt} mice.

FIG. 11B shows adipose tissue volumes on Mill in WT, Adcy9^{Gt/Gt}, CETP^{WT} and CETP^{Gt} mice.

### DETAILED DESCRIPTION OF THE INVENTION

### Definitions

An “effective amount,” when used in connection with a CETP inhibitor and an ADCY inhibitor, is the total amount of CETP inhibitor and ADCY inhibitor that is effective for treating or preventing a cardiovascular disorder or reducing risk of a cardiovascular event in a subject.

A “gene” is an ordered sequence of nucleotides located in a particular position on a particular chromosome that encodes a specific functional product and may include untranslated and untranscribed sequences in proximity to the coding regions. Such non-coding sequences may contain regulatory sequences needed for transcription and translation of the sequence or introns etc. or may as yet to have any function attributed to them beyond the occurrence of the SNP of interest.

The term “genotyping” refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, at rs 1967309 the nucleotides may be an A in some individuals and a G in other individuals. Those individuals who have an A at the position have the A allele and those who have a G have the G allele. In a diploid organism the individual will have two copies of the sequence containing the polymorphic position so the individual may have an A allele and a G allele or alternatively, two copies of the A alleles or two copies of the G allele. Those individuals who have two copies of the G allele are homozygous for the G allele, those individuals who have two copies of the A allele are homozygous for the A allele, and those individuals who have one copy of each allele are heterozygous. The alleles are often referred to as the A allele, often the major allele, and the B allele, often the minor allele. The genotypes may be AA (homozygous A), BB (homozygous B) or AB (heterozygous).

The term “about” when used in connection with a referenced numeric indication means the referenced numeric indication plus or minus up to 10% of that referenced numeric indication. For example, the language “about 50” means from 45 to 55.

The term “subject,” as used herein unless otherwise defined, is a mammal, e.g., a human, mouse, rat, guinea pig, dog, cat, horse, cow, pig, or non-human primate, such as a monkey, chimpanzee, or baboon. In some embodiments, the subject is a human. In some embodiments, the subject is an adult human. In some embodiments, the subject is a pediatric human.

As used herein, the term “adult human” refers to a human that is 18 years or older.

As used herein, the term “pediatric human” refers to a human that is 1 year to 18 years old.

### CEPT Inhibitors

CETP inhibitors that are useful in the compositions and methods of the invention include small molecules, anti-CETP antibodies and peptides that inhibit or suppress CETP activity.

CETP inhibitors that are useful in the compositions and methods of the invention include, but are not limited to, dalcetrapib, anacetrapib, evacetrapib, torcetrapib, BAY 60-5521, obicetrapib, BMS-795311, CP-800,569, DRL-17822, JNJ-28545595, JNJ-28614872, BAY 19-4789, BAY 38-1315, DLB S-1449 (Dexa Medica) and ATH-03 (Affris), and pharmaceutically acceptable salts of any of the foregoing.

“Dalcetrapib” refers to S-[2-({[1-(2-Ethylbutyl)cyclohexyl]carbonyl}amino)phenyl]-2-methylpropanethioate, and is also known as JTT-705 or CAS 211513-37-0. Dalcetrapib has the structure:

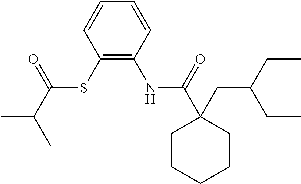 [SMILES: CCC(CC)CC1(C(=O)Nc2ccccc2SC(=O)C(C)C)CCCCC1]

“Anacetrapib” refers to (4 S, 5R)-5-[3,5-bi s(trifluoromethyl)phenyl]-3-{[4′-fluoro-2′-methoxy-5′-(propan-2-yl)-4-(trifluoromethyl)[1,1′-biphenyl]-2-yl]methyl}-4-methyl-1,3-oxazolidin-2-one, and is also known as (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one; MK-0859; or CAS 875446-37-0. Anacetrapib has the structure:

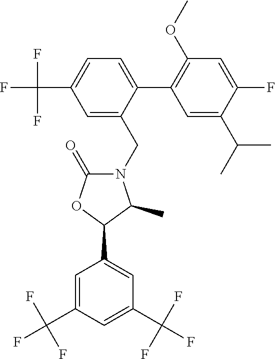 [SMILES: COc1cc(F)c(C(C)C)cc1-c1ccc(C(F)(F)F)cc1CN1C(=O)O[C@H](c2cc(C(F)(F)F)cc(C(F)(F)F)c2)[C@@H]1C]

“Evacetrapib” refers to trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl)cyclohexanecarboxylic acid, and is also known as LY2484595 or CAS 1186486-62-3. Evacetrapib has the structure:

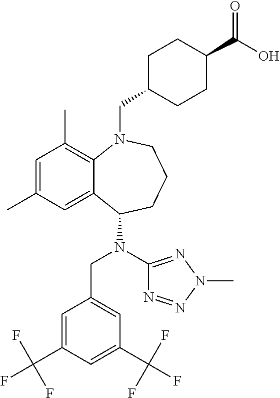 [SMILES: Cc1cc(C)c2c(c1)[C@@H](N(Cc1cc(C(F)(F)F)cc(C(F)(F)F)c1)c1nnn(C)n1)CCCN2C[C@H]1CC[C@H](C(=O)O)CC1]

“Torcetrapib” refers to (2R,4S)-4-[(3,5-bistrifluoromethylbenzyl) methoxycarbonylamino]-2-ethyl-6-trifluoromethyl-3,4-dihydro-2H-quinoline-1-carboxylic acid ethyl ester, and is also known as Ethyl (2R,4S)-4-({[3,5-bis(trifluoromethyl)phenyl]methyl}(methoxycarbonyl)amino)-2-ethyl-6-(trifluoromethyl)-1,2,3,4-tetrahydroquinoline-1-carboxylate; CP-529,414; or CAS 262352-17-0. Torcetrapib has the structure:

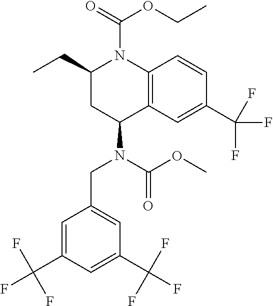 [SMILES: CCOC(=O)N1c2ccc(C(F)(F)F)cc2[C@@H](N(Cc2cc(C(F)(F)F)cc(C(F)(F)F)c2)C(=O)OC)C[C@H]1CC]

“BAY 60-5521” refers to (S)-4-cyclohexyl-2-cyclopentyl-3-((S)-fluoro(4-(trifluoromethyl)phenyl)methyl)-7,7-dimethyl-5,6,7,8-tetrahydroquinolin-5-ol, and is also known as CAS 893409-49-9. BAY 60-5521 has the structure:

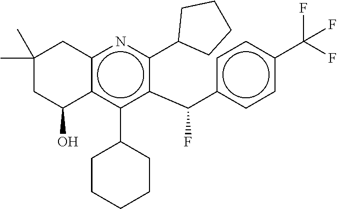

“Obicetrapib” refers to 4-((2-((3,5-bis(trifluoromethyl)benzyl)((2R,4S)-1-(ethoxycarbonyl)-2-ethyl-6-(trifluoromethyl)-1,2,3,4-tetrahydroquinolin-4-yl)amino)pyrimidin-5-yl)oxy)butanoic acid, and is also known as AMG-899, DEZ-001, TA-8995 or CAS 866399-87-3. Obicetrapib has the structure:

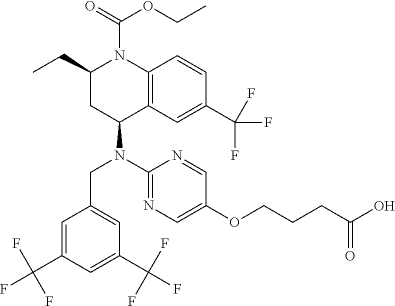 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

“BMS795311” refers to (R)—N-(1-(3-cyclopropoxy-4-fluorophenyl)-1-(3-fluoro-5-(2,2,3,3-tetrafluoropropanoyl)phenyl)-2-phenylethyl)-4-fluoro-3-(trifluoromethyl)benzamide, and is also known as CAS 939390-99-5. BMS795311 has the structure:

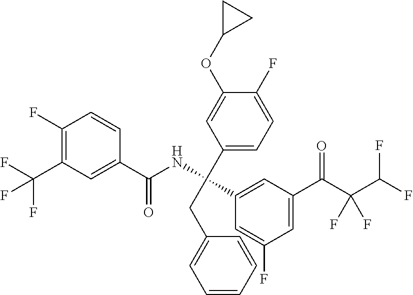 [SMILES: O=C(N[C@@](Cc1ccccc1)(c1cc(F)cc(C(=O)C(F)(F)C(F)F)c1)c1ccc(F)c(OC2CC2)c1)c1ccc(F)c(C(F)(F)F)c1]

“CP-800,569” refers to (2R)-3-[3-(4-chloro-3-ethylphenoxy)-n-[[3-(1,1,2,2-tetrafluoroethoxy)phenyl]methyl]anilino]-1,1,1-trifluoropropan-2-ol . CP-800,569 has the structure:

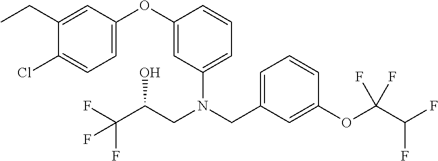 [SMILES: CCc1cc(Oc2cccc(N(Cc3cccc(OC(F)(F)C(F)F)c3)C[C@@H](O)C(F)(F)F)c2)ccc1Cl]

“DRL-17822” refers to CAS 1454689-50-9 and is disclosed in WO 2014128564 and WO 2014076568. DRL-17822 has the structure:

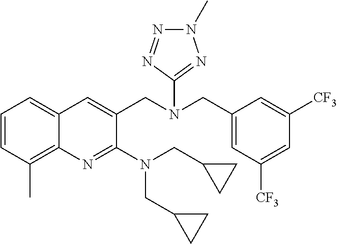 [SMILES: Cc1cccc2cc(CN(Cc3cc(C(F)(F)F)cc(C(F)(F)F)c3)c3nnn(C)n3)c(N(CC3CC3)CC3CC3)nc12]

“JNJ-28545595” refers to 1,1,1-Trifluoro-3-[2-[3-(1,1,2,2-tetra-fluoroethoxy)phenyl]-5-(3-trifluoromethoxyphenyl)-3,4-dihydro-2H-quinolin-1-yl]-propan-2-ol.

“JNJ-28614872” refers to 1,1,1-Trifluoro-3-[3-[3-(1,1,2,2-tetrafluoro-ethoxy)-phenyl]-8-(3-trifluoromethoxy-phenyl)-2,3-dihydro-benzo[1,4]oxazin-4-yl]-propan-2-ol.

The structure of JNJ-28545595 and JNJ-28614872 is set forth below:

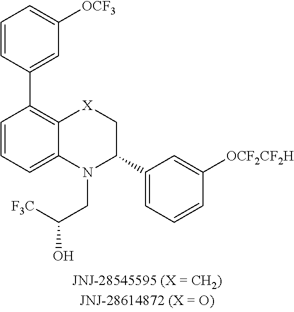

The structure of “BAY 19-4789” and “BAY 38-1315” is set forth below:

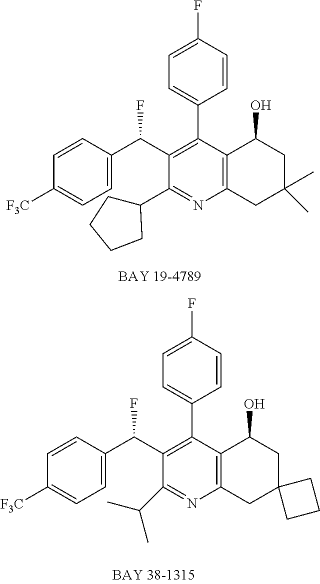 [SMILES: CC1(C)Cc2nc(C3CCCC3)c([C@@H](F)c3ccc(C(F)(F)F)cc3)c(-c3ccc(F)cc3)c2[C@@H](O)C1 ; CC(C)c1nc2c(c(-c3ccc(F)cc3)c1[C@@H](F)c1ccc(C(F)(F)F)cc1)[C@@H](O)CC1(CCC1)C2]

Additional CETP inhibitors useful in the compositions and methods of the invention include those disclosed in WO 2016/086453 or Chen et al., European Journal of Medicinal Chemistry, (2017) 139:201-213, and have the structure:

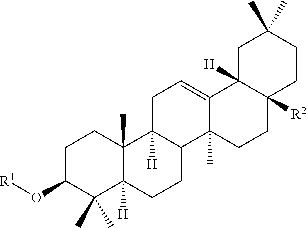

| R¹ | R² |
|---|---|
| H | —CO₂H |
| —COCH₃ | —CO₂H |
| —COCH₂CH₃ | —CO₂H |
| —CO(CH₂)₂CH₃ | —CO₂H |
| —CO(CH₂)₇CH₃ | —CO₂H |
| —CO(CH₂)₁₄CH₃ | —CO₂H |
| 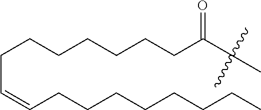 [SMILES: *C(=O)CCCCCCC/C=C\CCCCCCCC] | —CO₂H |
| 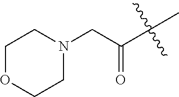 [SMILES: *C(=O)CN1CCOCC1] | —CO₂H |
| 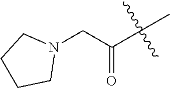 [SMILES: *C(=O)CN1CCCC1] | —CO₂H |
| 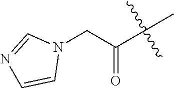 [SMILES: *C(=O)Cn1ccnc1] | —CO₂H |
| 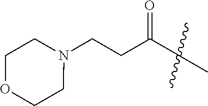 [SMILES: *C(=O)CCN1CCOCC1] | —CO₂H |
| 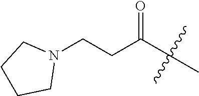 [SMILES: *C(=O)CCN1CCCC1] | —CO₂H |
| 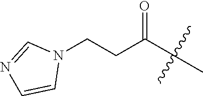 [SMILES: *C(=O)CCn1ccnc1] | —CO₂H |
| —CO(CH₂)₂CO₂H | —CH₃ |
| —CO(CH₂)₃CO₂H | —CH₃ |
| —CO(CH₂)₂CO₂H | —CO₂H |
| —CO(CH₂)₃CO₂H | —CO₂H |
| —CO(CH₂)₄CO₂H | —CO₂H |
| 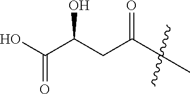 [SMILES: *C(=O)C[C@H](O)C(=O)O] | —CO₂H |
| —CO(CH₂)₂CONH₂ | —CO₂H |
| —CO(CH₂)₂CON(CH₃)₂ | —CO₂H |
| 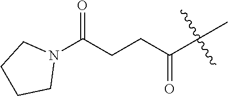 [SMILES: *C(=O)CCC(=O)N1CCCC1] | —CO₂H |
| 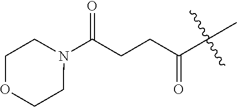 [SMILES: *C(=O)CCC(=O)N1CCOCC1] | —CO₂H |
| 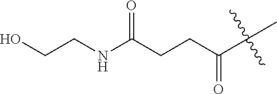 [SMILES: *C(=O)CCC(=O)NCCO] | —CO₂H |
| 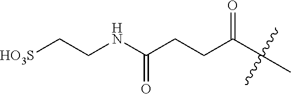 [SMILES: *C(=O)CCC(=O)NCCS(=O)(=O)O] | —CO₂H |
| —CO(CH₂)₃CONH₂ | —CO₂H |
| —CO(CH₂)₃CON(CH₃)₂ | —CO₂H |
| 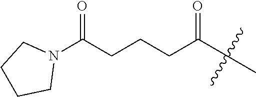 [SMILES: *C(=O)CCCC(=O)N1CCCC1] | —CO₂H |
| 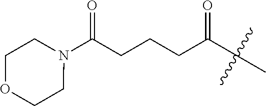 [SMILES: *C(=O)CCCC(=O)N1CCOCC1] | —CO₂H |
| 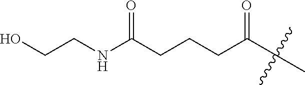 [SMILES: *C(=O)CCCC(=O)NCCO] | —CO₂H |
| 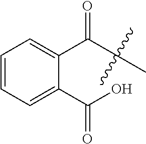 [SMILES: *C(=O)c1ccccc1C(=O)O] | —CO₂H |
| —CO(CH₂)₃CO₂H | —CO₂CH₂CO₂H |
| —CO(CH₂)₃CO₂H | —CO₂CH₃ |
| H | —CONH₂ |
| H | —CO₂CH₂CO₂H |

and pharmaceutically acceptable salts of the foregoing;

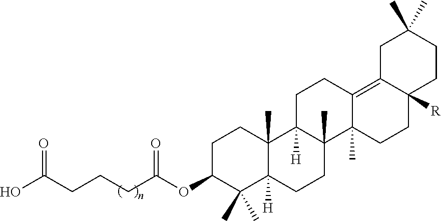

| n | R |
|---|---|
| 0 | —CO₂H |
| 0 | —CO₂CH₃ |
| 1 | —CO₂H |
| 1 | —CO₂CH₃ |
| 2 | —CO₂H |
| 2 | —CO₂CH₃ |

and pharmaceutically acceptable salts of the foregoing;

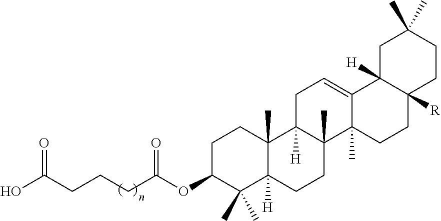

| n | R |
|---|---|
| 0 | —CONH₂ |
| 0 | —CON(CH₃)₂ |
| 1 | —CONH₂ |
| 1 | —CON(CH₃)₂ |
| 1 | —CONHCH₂CO₂H |
| 1 | —CONHCH₂CO₂CH₃ |
| 1 | —COCH₂CO₂H |
| 2 | —CONH₂ |
| 2 | —CON(CH₃)₂ |
| 2 | —CONHCH₂CO₂H |
| 2 | —CONHCH₂CO₂CH₃ |

and pharmaceutically acceptable salts of the foregoing;

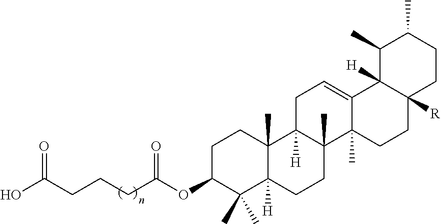

| n | R |
|---|---|
| 1 | —CO₂H |
| 1 | —CO₂CH₃ |
| 2 | —CO₂H |
| 2 | —CO₂CH₃ |
| 0 | —CON(CH₃)₂ |

and pharmaceutically acceptable salts of the foregoing;

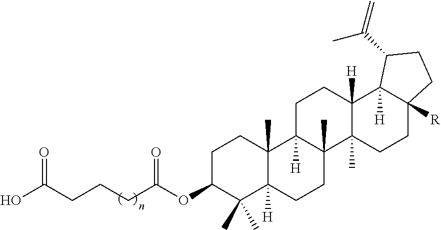

| n | R |
|---|---|
| 0 | —CON(CH₃)₂ |
| 0 | —CONH₂ |
| 1 | —CO₂H |
| 2 | —CO₂H |
| 2 | —CO₂CH₃ |

and pharmaceutically acceptable salts of the foregoing;

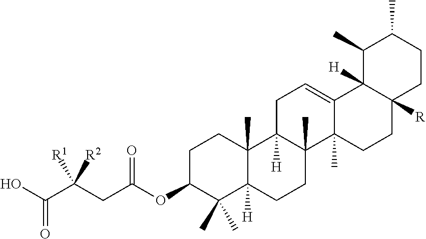

| R¹ | R² | R |
|---|---|---|
| H | OH | CH₃ |
| CH₃ | CH₃ | CH₃ |
| H | OH | —CO₂H |
| H | OH | —CONH₂ |
| CH₃ | CH₃ | —CONH₂ |
| H | OH | —CON(CH₃)₂ |
| CH₃ | CH₃ | —CON(CH₃)₂ |

and pharmaceutically acceptable salts of the foregoing;

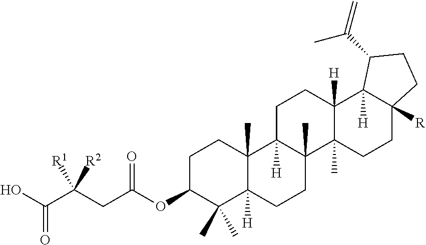

| R1 | R2 | R |
|---|---|---|
| H | OH | CH₃ |
| H | OH | —CO₂H |
| H | OH | —CONH₂ |
| CH₃ | CH₃ | —CONH₂ |
| H | OH | —CON(CH₃)₂ |
| CH₃ | CH₃ | —CON(CH₃)₂ |

and pharmaceutically acceptable salts of the foregoing;

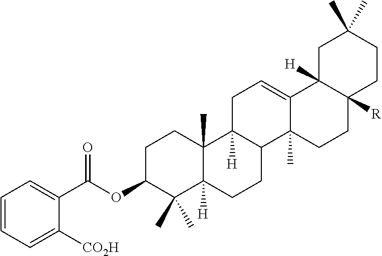

R
—CO₂CH₃
—CONH₂
—CON(CH₃)₂
—CONHCH₂CO₂H
—CONHCH₂CO₂CH₃

and pharmaceutically acceptable salts of the foregoing; and

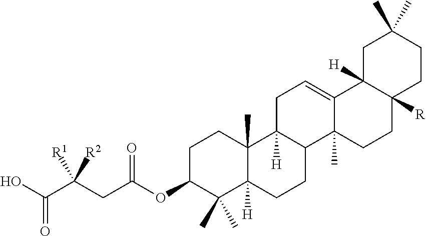

| R¹ | R² | R |
|---|---|---|
| H | OH | CH₃ |
| CH₃ | CH₃ | CH₃ |
| H | OH | —CONH₂ |
| CH₃ | CH₃ | —CONH₂ |
| H | OH | —CON(CH₃)₂ |
| CH₃ | CH₃ | —CON(CH₃)₂ |

and pharmaceutically acceptable salts of the foregoing.

Additional CETP inhibitors useful in the compositions and methods of the invention are disclosed in WO 2016/086453 or Chen et al. and include, but are not limited to:

Structure

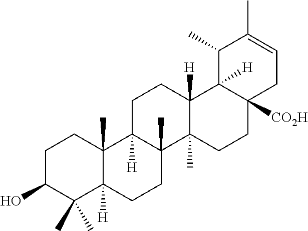

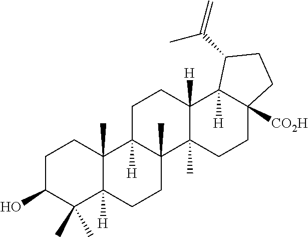

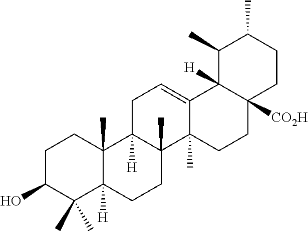

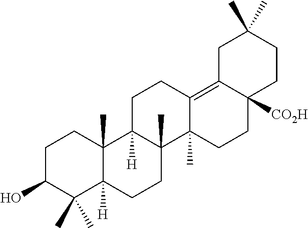

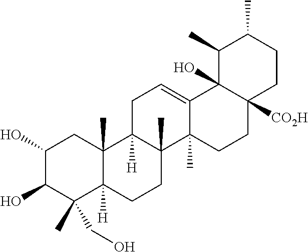

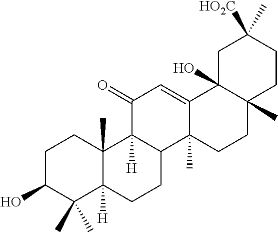

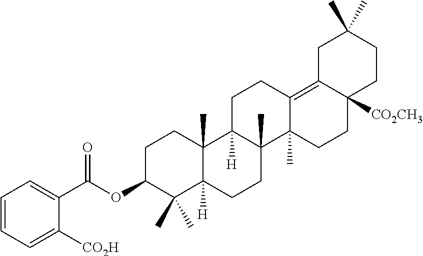

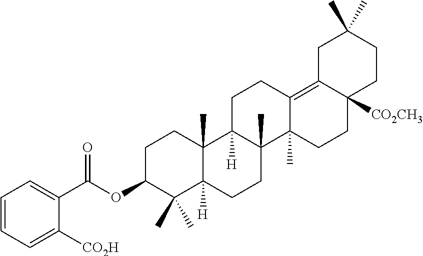

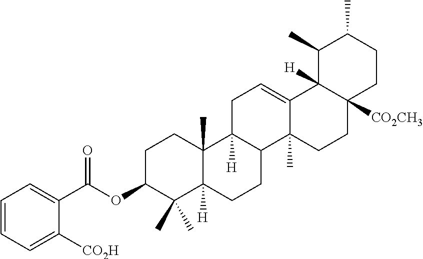

and pharmaceutically acceptable salts of the foregoing.

Further CETP inhibitors useful in the compositions and methods of the invention include those disclosed in WO 2017/011279, and have the structure:

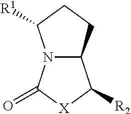

| X | R¹ | R² |
|---|---|---|
| S | 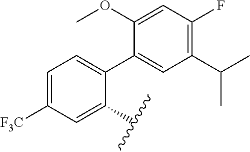 | 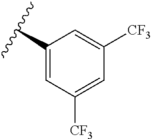 |
| S | 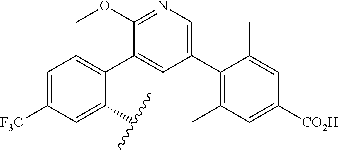 | 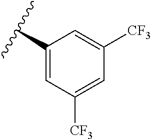 |
| S | 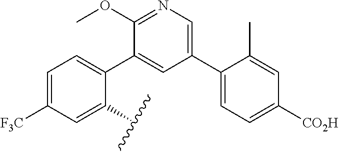 | 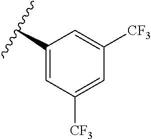 |
| S | 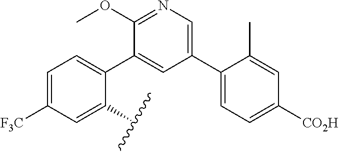 | 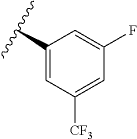 |
| S | 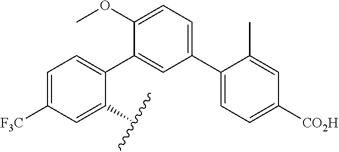 | 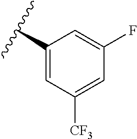 |
| S | 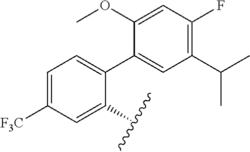 | 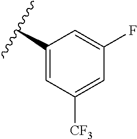 |
| S | 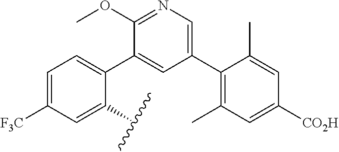 | 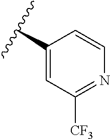 |
| S | 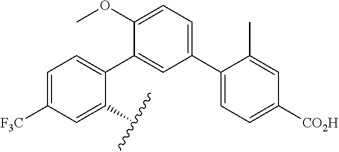 | 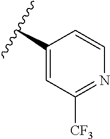 |
| S | 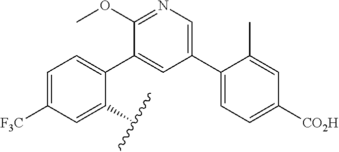 | 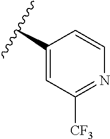 |
| S | 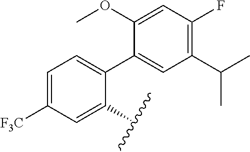 | 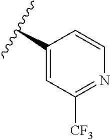 |
| S | 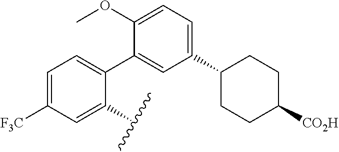 | 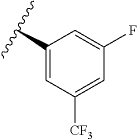 |
| S | 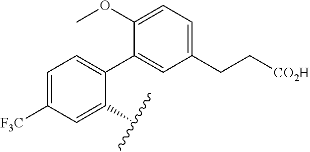 | 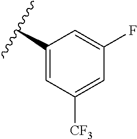 |
| CH₂ | 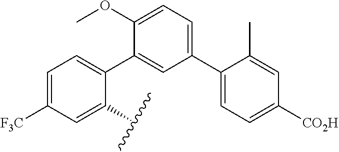 | 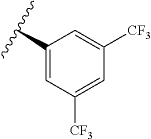 |
| CH₂ | 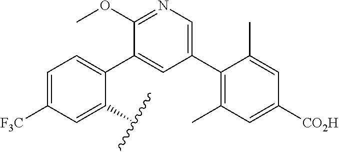 | 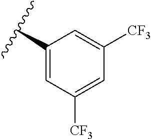 |
| CH₂ | 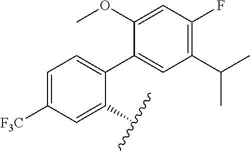 | 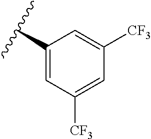 |
| CH₂ | 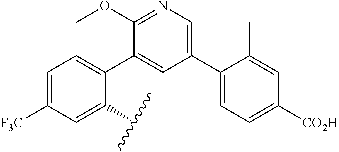 | 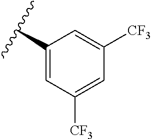 |
| CH₂ | 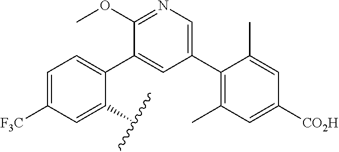 | 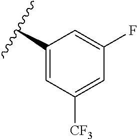 |
| CH₂ | 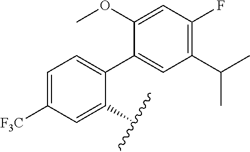 | 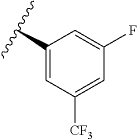 |
| CH₂ | 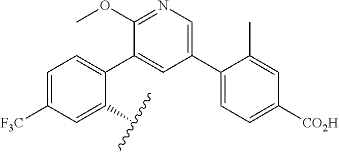 | 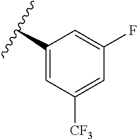 |
| CH₂ | 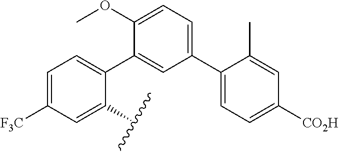 | 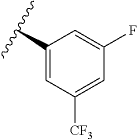 |
| CH₂ | 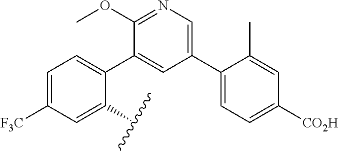 | 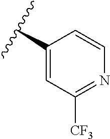 |

and pharmaceutically acceptable salts of the foregoing.

Still other CETP inhibitors useful in the compositions and methods of the invention include those disclosed in WO 2016/018729, and have a structure according to the following:

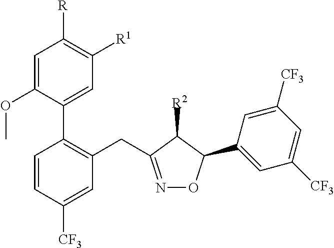

| R | R¹ | R² |
|---|---|---|
| F | 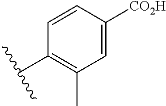 [SMILES: *c1ccc(C(=O)O)cc1C] | H |
| F | 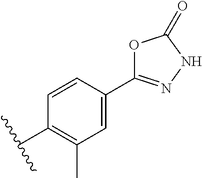 [SMILES: *c1ccc(-c2n[nH]c(=O)o2)cc1C] | H |
| H | 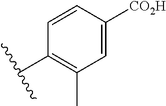 [SMILES: *c1ccc(C(=O)O)cc1C] | CH₃ |
| F | 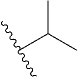 [SMILES: *C(C)C] | H |
| H | 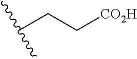 [SMILES: *CCC(=O)O] | H |

and pharmaceutically acceptable salts of the foregoing;

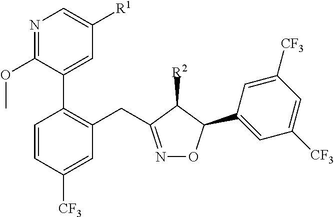

| R¹ | R² |
|---|---|
| 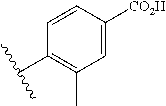 [SMILES: *c1ccc(C(=O)O)cc1C] | H |
| 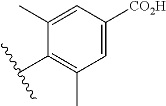 [SMILES: *c1c(C)cc(C(=O)O)cc1C] | CH₃ |
| 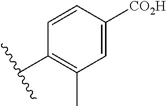 [SMILES: *c1ccc(C(=O)O)cc1C] | CH₃ |

and pharmaceutically acceptable salts of the foregoing;

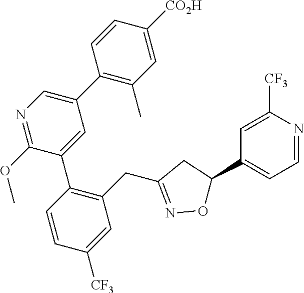 [SMILES: COc1ncc(-c2ccc(C(=O)O)cc2C)cc1-c1ccc(C(F)(F)F)cc1CC1=NO[C@H](c2ccnc(C(F)(F)F)c2)C1]

and pharmaceutically acceptable salts thereof;

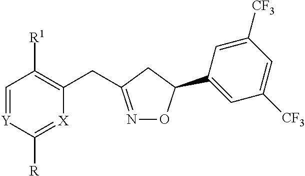

| X | Y | R | R¹ |
|---|---|---|---|
| CH | N | CF₃ | 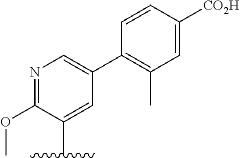 [SMILES: *c1cc(-c2ccc(C(=O)O)cc2C)cnc1OC] |
| CH | N | CF₃ | 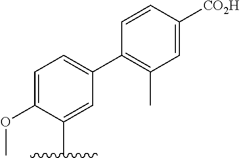 [SMILES: *c1cc(-c2ccc(C(=O)O)cc2C)ccc1OC] |
| N | CH | CF₃ | 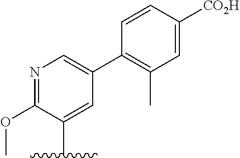 [SMILES: *c1cc(-c2ccc(C(=O)O)cc2C)cnc1OC] |
| N | CH | OCH₃ | 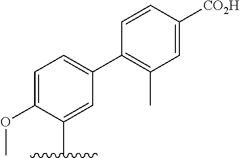 [SMILES: *c1cc(-c2ccc(C(=O)O)cc2C)ccc1OC] |
| N | CH | OCH₃ | 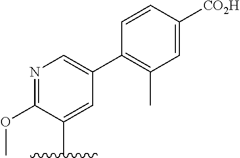 [SMILES: *c1cc(-c2ccc(C(=O)O)cc2C)cnc1OC] |

and pharmaceutically acceptable salts of the foregoing; and

| 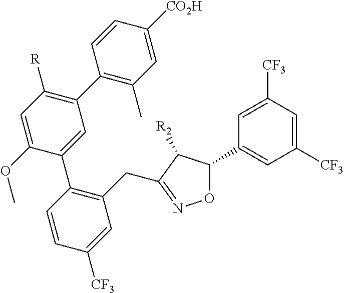 | |
|---|---|
| R | R² |
| F | H |
| H | CH₃ |

and pharmaceutically acceptable salts of the foregoing.

Additional CETP inhibitors useful in the compositions and methods of the invention are disclosed in U.S. Pat. No. 7,781,426, including, but not limited to:

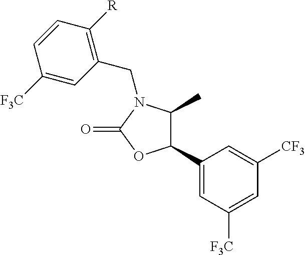

R

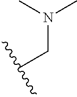 [SMILES: *CN(C)C]

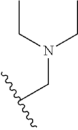 [SMILES: *CN(CC)CC]

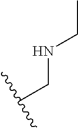 [SMILES: *CNCC]

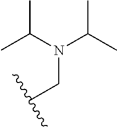 [SMILES: *CN(C(C)C)C(C)C]

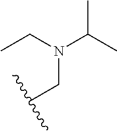 [SMILES: *CN(CC)C(C)C]

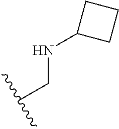 [SMILES: *CNC1CCC1]

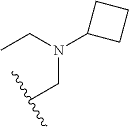 [SMILES: *CN(CC)C1CCC1]

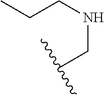 [SMILES: *CNCCC]

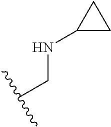 [SMILES: *CNC1CC1]

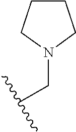 [SMILES: *CN1CCCC1]

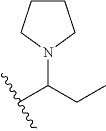 [SMILES: *C(CC)N1CCCC1]

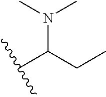 [SMILES: *C(CC)N(C)C]

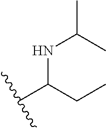 [SMILES: *C(CC)NC(C)C]

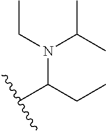 [SMILES: *C(CC)N(CC)C(C)C]

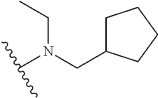 [SMILES: *N(CC)CC1CCCC1]

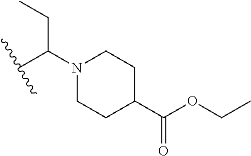 [SMILES: *C(CC)N1CCC(C(=O)OCC)CC1]

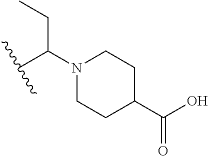 [SMILES: *C(CC)N1CCC(C(=O)O)CC1]

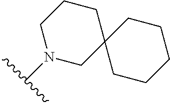 [SMILES: *N1CCCC2(CCCCC2)C1]

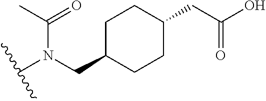 [SMILES: *N(C[C@H]1CC[C@H](CC(=O)O)CC1)C(C)=O]

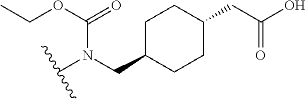 [SMILES: *N(C[C@H]1CC[C@H](CC(=O)O)CC1)C(=O)OCC]

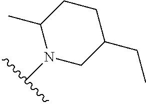 [SMILES: *N1CC(CC)CCC1C]

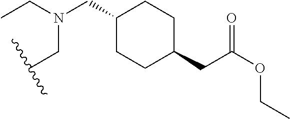 [SMILES: *CN(CC)C[C@H]1CC[C@H](CC(=O)OCC)CC1]

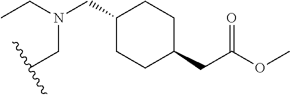 [SMILES: *CN(CC)C[C@H]1CC[C@H](CC(=O)OC)CC1]

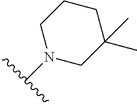 [SMILES: *N1CCCC(C)(C)C1]

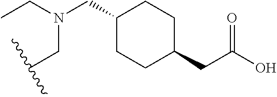 [SMILES: *CN(CC)C[C@H]1CC[C@H](CC(=O)O)CC1]

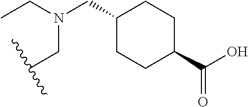 [SMILES: *CN(CC)C[C@H]1CC[C@H](C(=O)O)CC1]

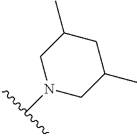 [SMILES: *N1CC(C)CC(C)C1]

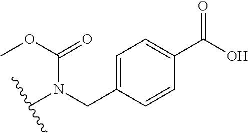 [SMILES: *N(Cc1ccc(C(=O)O)cc1)C(=O)OC]

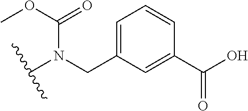 [SMILES: *N(Cc1cccc(C(=O)O)c1)C(=O)OC]

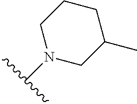 [SMILES: *N1CCCC(C)C1]

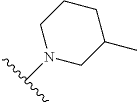 [SMILES: *N1CCCC(C)C1]

and pharmaceutically acceptable salts of the foregoing;

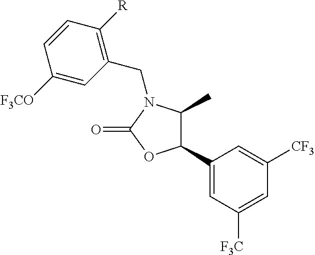

R

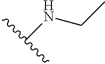 [SMILES: *NCC]

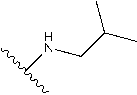 [SMILES: *NCC(C)C]

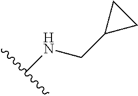 [SMILES: *NCC1CC1]

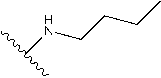 [SMILES: *NCCCC]

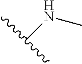 [SMILES: *NC]

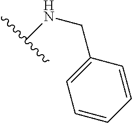 [SMILES: *NCc1ccccc1]

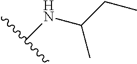 [SMILES: *NC(C)CC]

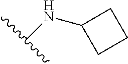 [SMILES: *NC1CCC1]

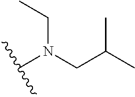 [SMILES: *N(CC)CC(C)C]

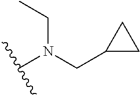 [SMILES: *N(CC)CC1CC1]

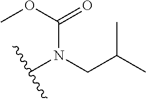 [SMILES: *N(CC(C)C)C(=O)OC]

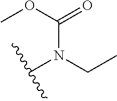 [SMILES: *N(CC)C(=O)OC]

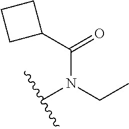 [SMILES: *N(CC)C(=O)C1CCC1]

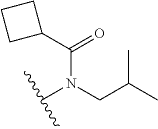 [SMILES: *N(CC(C)C)C(=O)C1CCC1]

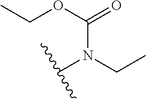 [SMILES: *N(CC)C(=O)OCC]

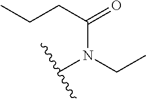 [SMILES: *N(CC)C(=O)CCC]

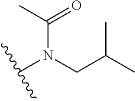 [SMILES: *N(CC(C)C)C(C)=O]

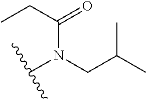 [SMILES: *N(CC(C)C)C(=O)CC]

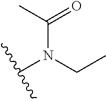 [SMILES: *N(CC)C(C)=O]

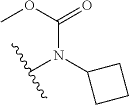 [SMILES: *N(C(=O)OC)C1CCC1]

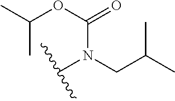 [SMILES: *N(CC(C)C)C(=O)OC(C)C]

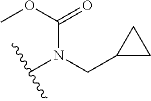 [SMILES: *N(CC1CC1)C(=O)OC]

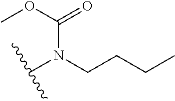 [SMILES: *N(CCCC)C(=O)OC]

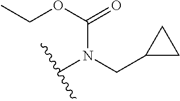 [SMILES: *N(CC1CC1)C(=O)OCC]

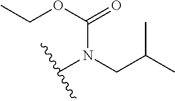 [SMILES: *N(CC(C)C)C(=O)OCC]

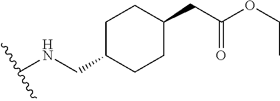 [SMILES: *NC[C@H]1CC[C@H](CC(=O)OCC)CC1]

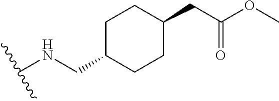 [SMILES: *NC[C@H]1CC[C@H](CC(=O)OC)CC1]

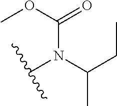 [SMILES: *N(C(=O)OC)C(C)CC]

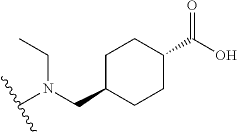 [SMILES: *N(CC)C[C@H]1CC[C@H](C(=O)O)CC1]

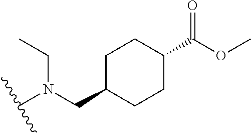 [SMILES: *N(CC)C[C@H]1CC[C@H](C(=O)OC)CC1]

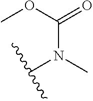 [SMILES: *N(C)C(=O)OC]

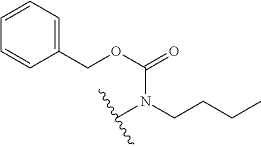 [SMILES: *N(CCCC)C(=O)OCc1ccccc1]

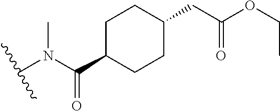 [SMILES: *N(C)C(=O)[C@H]1CC[C@H](CC(=O)OCC)CC1]

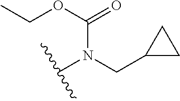 [SMILES: *N(CC1CC1)C(=O)OCC]

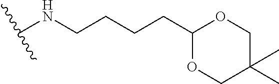 [SMILES: *NCCCCC1OCC(C)(C)CO1]

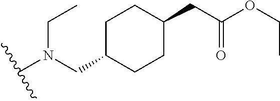 [SMILES: *N(CC)C[C@H]1CC[C@H](CC(=O)OCC)CC1]

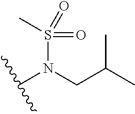 [SMILES: *N(CC(C)C)S(C)(=O)=O]

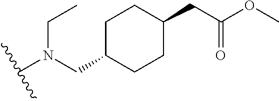 [SMILES: *N(CC)C[C@H]1CC[C@H](CC(=O)OC)CC1]

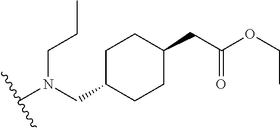 [SMILES: *N(CCC)C[C@H]1CC[C@H](CC(=O)OCC)CC1]

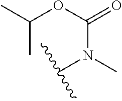 [SMILES: *N(C)C(=O)OC(C)C]

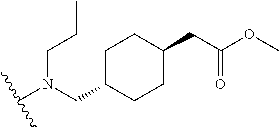 [SMILES: *N(CCC)C[C@H]1CC[C@H](CC(=O)OC)CC1]

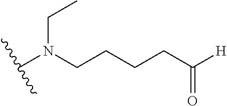 [SMILES: *N(CC)CCCCC=O]

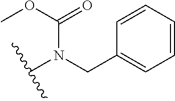 [SMILES: *N(Cc1ccccc1)C(=O)OC]

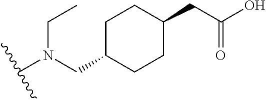 [SMILES: *N(CC)C[C@H]1CC[C@H](CC(=O)O)CC1]

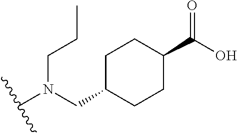 [SMILES: *N(CCC)C[C@H]1CC[C@H](C(=O)O)CC1]

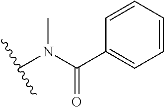 [SMILES: *N(C)C(=O)c1ccccc1]

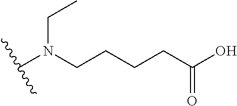 [SMILES: *N(CC)CCCCC(=O)O]

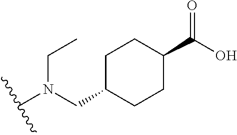 [SMILES: *N(CC)C[C@H]1CC[C@H](C(=O)O)CC1]

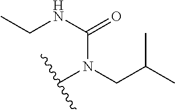 [SMILES: *N(CC(C)C)C(=O)NCC]

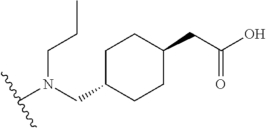 [SMILES: *N(CCC)C[C@H]1CC[C@H](CC(=O)O)CC1]

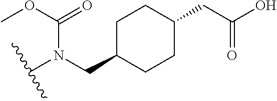 [SMILES: *N(C[C@H]1CC[C@H](CC(=O)O)CC1)C(=O)OC]

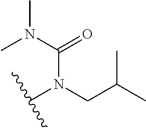 [SMILES: *N(CC(C)C)C(=O)N(C)C]

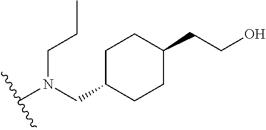 [SMILES: *N(CCC)C[C@H]1CC[C@H](CCO)CC1]

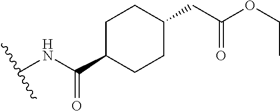 [SMILES: *NC(=O)[C@H]1CC[C@H](CC(=O)OCC)CC1]

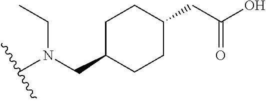 [SMILES: *N(CC)C[C@H]1CC[C@H](CC(=O)O)CC1]

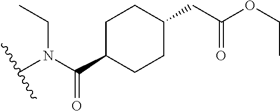 [SMILES: *N(CC)C(=O)[C@H]1CC[C@H](CC(=O)OCC)CC1]

and pharmaceutically acceptable salts of the foregoing;

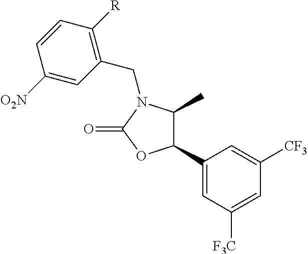

R

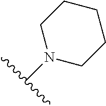 [SMILES: *N1CCCCC1]

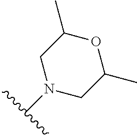 [SMILES: *N1CC(C)OC(C)C1]

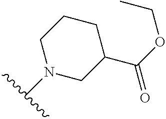 [SMILES: *N1CCCC(C(=O)OCC)C1]

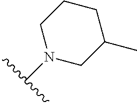 [SMILES: *N1CCCC(C)C1]

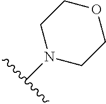 [SMILES: *N1CCOCC1]

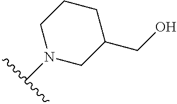 [SMILES: *N1CCCC(CO)C1]

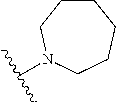 [SMILES: *N1CCCCCC1]

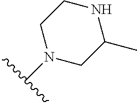 [SMILES: *N1CCNC(C)C1]

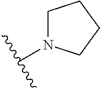 [SMILES: *N1CCCC1]

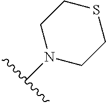 [SMILES: *N1CCSCC1]

and pharmaceutically acceptable salts of the foregoing; and

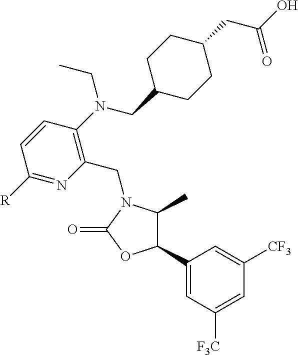

R
H

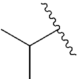 [SMILES: *C(C)C]

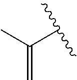 [SMILES: *C(=C)C]

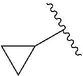 [SMILES: *C1CC1]

and pharmaceutically acceptable salts of the foregoing.

Additional CETP inhibitors useful in the compositions and methods of the invention are disclosed in U.S. Pat. No. 7,652,049, including, but not limited to:

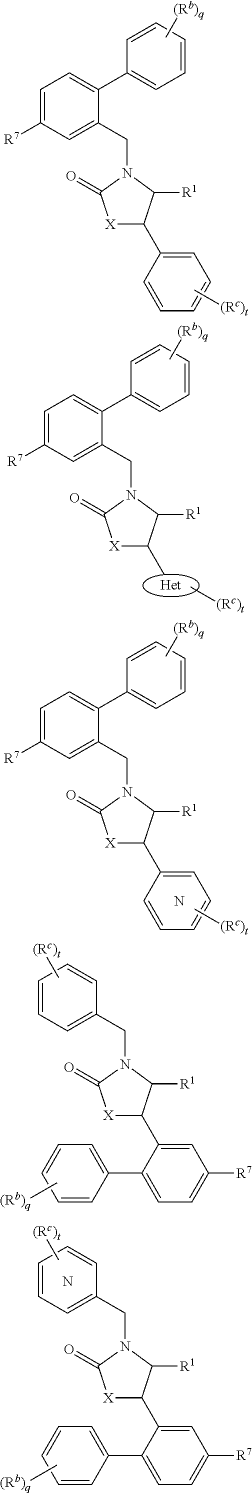

and pharmaceutically acceptable salts of the foregoing;

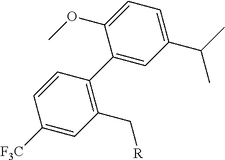

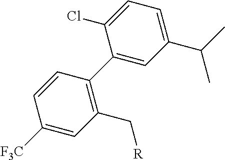

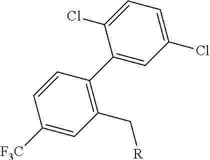

R

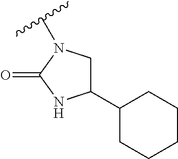 [SMILES: *N1CC(C2CCCCC2)NC1=O]

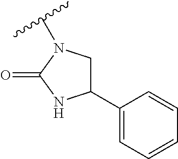 [SMILES: *N1CC(c2ccccc2)NC1=O]

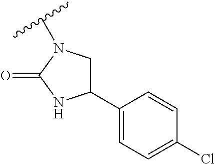 [SMILES: *N1CC(c2ccc(Cl)cc2)NC1=O]

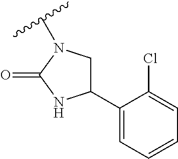 [SMILES: *N1CC(c2ccccc2Cl)NC1=O]

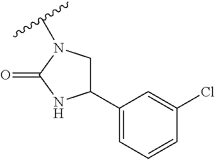 [SMILES: *N1CC(c2cccc(Cl)c2)NC1=O]

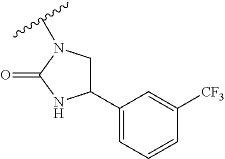 [SMILES: *N1CC(c2cccc(C(F)(F)F)c2)NC1=O]

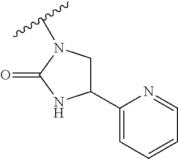 [SMILES: *N1CC(c2ccccn2)NC1=O]

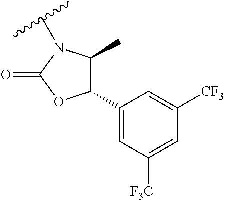 [SMILES: *N1C(=O)O[C@@H](c2cc(C(F)(F)F)cc(C(F)(F)F)c2)[C@@H]1C]

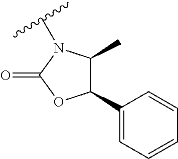 [SMILES: *N1C(=O)O[C@H](c2ccccc2)[C@@H]1C]

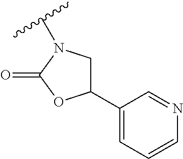 [SMILES: *N1CC(c2cccnc2)OC1=O]

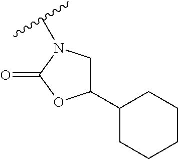 [SMILES: *N1CC(C2CCCCC2)OC1=O]

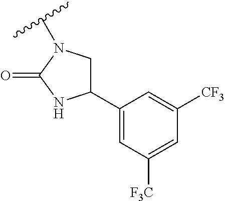 [SMILES: *N1CC(c2cc(C(F)(F)F)cc(C(F)(F)F)c2)NC1=O]

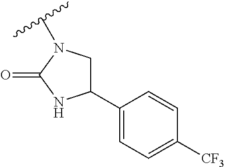 [SMILES: *N1CC(c2ccc(C(F)(F)F)cc2)NC1=O]

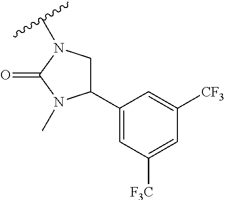 [SMILES: *N1CC(c2cc(C(F)(F)F)cc(C(F)(F)F)c2)N(C)C1=O]

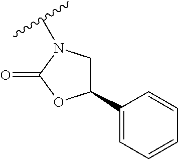 [SMILES: *N1C[C@@H](c2ccccc2)OC1=O]

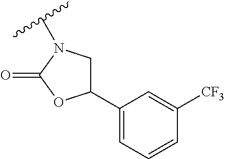 [SMILES: *N1CC(c2cccc(C(F)(F)F)c2)OC1=O]

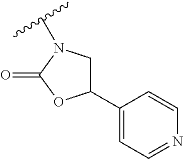 [SMILES: *N1CC(c2ccncc2)OC1=O]

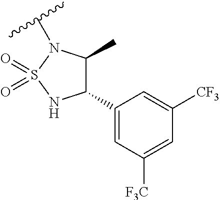 [SMILES: *N1[C@@H](C)[C@H](c2cc(C(F)(F)F)cc(C(F)(F)F)c2)NS1(=O)=O]

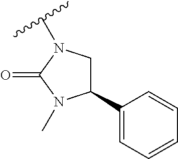 [SMILES: *N1C[C@@H](c2ccccc2)N(C)C1=O]

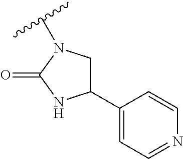 [SMILES: *N1CC(c2ccncc2)NC1=O]

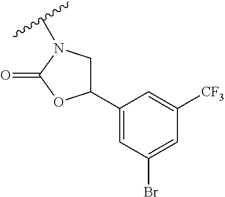 [SMILES: *N1CC(c2cc(Br)cc(C(F)(F)F)c2)OC1=O]

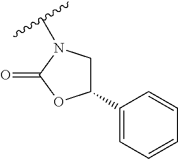 [SMILES: *N1C[C@H](c2ccccc2)OC1=O]

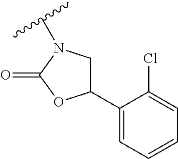 [SMILES: *N1CC(c2ccccc2Cl)OC1=O]

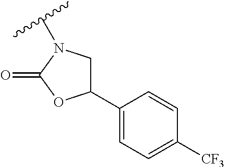 [SMILES: *N1CC(c2ccc(C(F)(F)F)cc2)OC1=O]

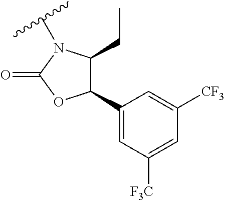 [SMILES: *N1C(=O)O[C@H](c2cc(C(F)(F)F)cc(C(F)(F)F)c2)[C@@H]1CC]

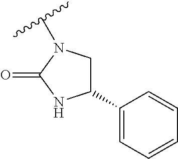

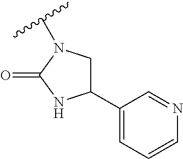 [SMILES: *N1CC(c2cccnc2)NC1=O]

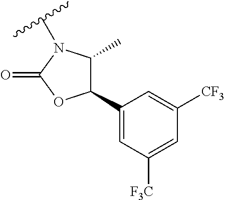 [SMILES: *N1C(=O)O[C@H](c2cc(C(F)(F)F)cc(C(F)(F)F)c2)[C@H]1C]

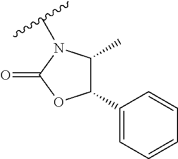 [SMILES: *N1C(=O)O[C@@H](c2ccccc2)[C@H]1C]

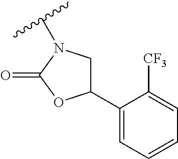 [SMILES: *N1CC(c2ccccc2C(F)(F)F)OC1=O]

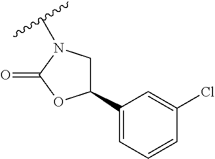 [SMILES: *N1C[C@@H](c2cccc(Cl)c2)OC1=O]

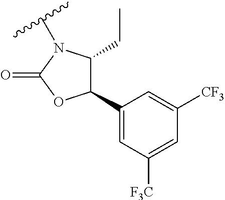 [SMILES: *N1C(=O)O[C@H](c2cc(C(F)(F)F)cc(C(F)(F)F)c2)[C@H]1CC]

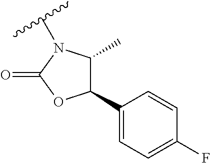 [SMILES: *N1C(=O)O[C@H](c2ccc(F)cc2)[C@H]1C]

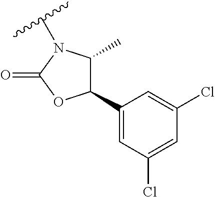 [SMILES: *N1C(=O)O[C@H](c2cc(Cl)cc(Cl)c2)[C@H]1C]

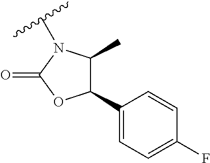 [SMILES: *N1C(=O)O[C@H](c2ccc(F)cc2)[C@@H]1C]

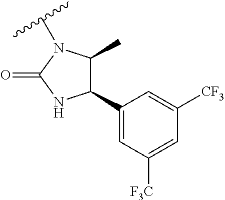 [SMILES: *N1C(=O)N[C@H](c2cc(C(F)(F)F)cc(C(F)(F)F)c2)[C@@H]1C]

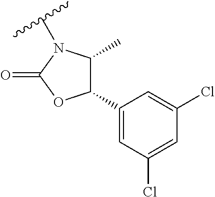 [SMILES: *N1C(=O)O[C@@H](c2cc(Cl)cc(Cl)c2)[C@H]1C]

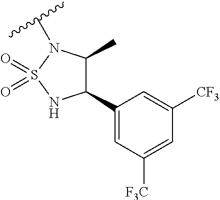 [SMILES: *N1[C@@H](C)[C@@H](c2cc(C(F)(F)F)cc(C(F)(F)F)c2)NS1(=O)=O]

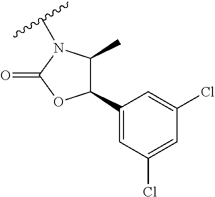 [SMILES: *N1C(=O)O[C@H](c2cc(Cl)cc(Cl)c2)[C@@H]1C]

and pharmaceutically acceptable salts of the foregoing;

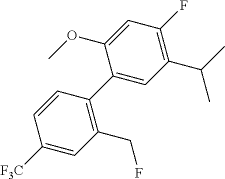 [SMILES: COc1cc(F)c(C(C)C)cc1-c1ccc(C(F)(F)F)cc1CF]

R

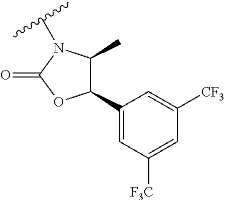 [SMILES: *N1C(=O)O[C@H](c2cc(C(F)(F)F)cc(C(F)(F)F)c2)[C@@H]1C]

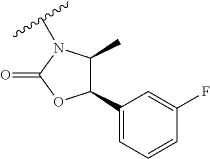 [SMILES: *N1C(=O)O[C@H](c2cccc(F)c2)[C@@H]1C]

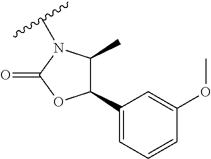 [SMILES: *N1C(=O)O[C@H](c2cccc(OC)c2)[C@@H]1C]

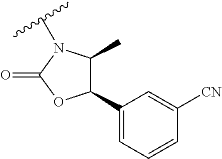 [SMILES: *N1C(=O)O[C@H](c2cccc(C#N)c2)[C@@H]1C]

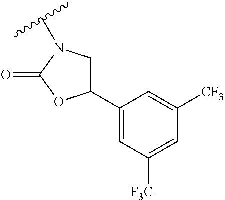 [SMILES: *N1CC(c2cc(C(F)(F)F)cc(C(F)(F)F)c2)OC1=O]

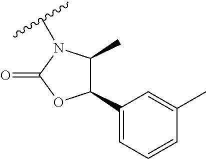 [SMILES: *N1C(=O)O[C@H](c2cccc(C)c2)[C@@H]1C]

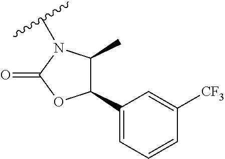 [SMILES: *N1C(=O)O[C@H](c2cccc(C(F)(F)F)c2)[C@@H]1C]

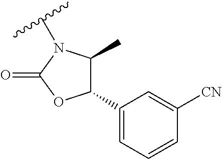 [SMILES: *N1C(=O)O[C@@H](c2cccc(C#N)c2)[C@@H]1C]

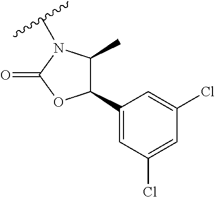 [SMILES: *N1C(=O)O[C@H](c2cc(Cl)cc(Cl)c2)[C@@H]1C]

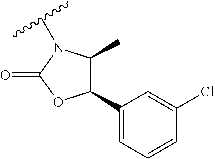 [SMILES: *N1C(=O)O[C@H](c2cccc(Cl)c2)[C@@H]1C]

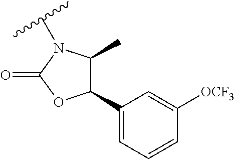 [SMILES: *N1C(=O)O[C@H](c2cccc(OC(F)(F)F)c2)[C@@H]1C]

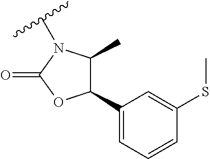 [SMILES: *N1C(=O)O[C@H](c2cccc(SC)c2)[C@@H]1C]

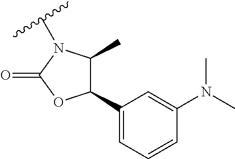 [SMILES: *N1C(=O)O[C@H](c2cccc(N(C)C)c2)[C@@H]1C]

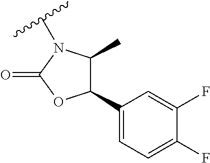 [SMILES: *N1C(=O)O[C@H](c2ccc(F)c(F)c2)[C@@H]1C]

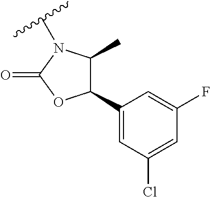 [SMILES: *N1C(=O)O[C@H](c2cc(F)cc(Cl)c2)[C@@H]1C]

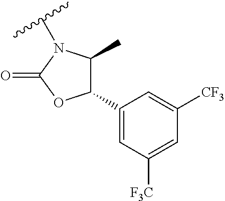 [SMILES: *N1C(=O)O[C@@H](c2cc(C(F)(F)F)cc(C(F)(F)F)c2)[C@@H]1C]

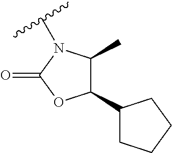 [SMILES: *N1C(=O)O[C@H](C2CCCC2)[C@@H]1C]

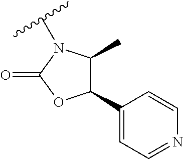 [SMILES: *N1C(=O)O[C@H](c2ccncc2)[C@@H]1C]

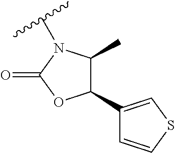 [SMILES: *N1C(=O)O[C@H](c2ccsc2)[C@@H]1C]

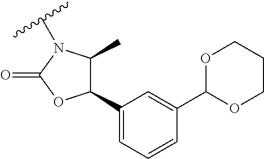 [SMILES: *N1C(=O)O[C@H](c2cccc(C3OCCCO3)c2)[C@@H]1C]

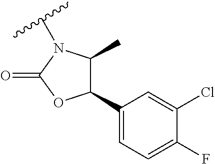 [SMILES: *N1C(=O)O[C@H](c2ccc(F)c(Cl)c2)[C@@H]1C]

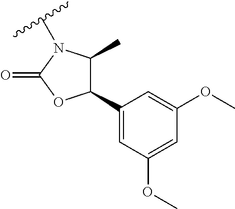 [SMILES: *N1C(=O)O[C@H](c2cc(OC)cc(OC)c2)[C@@H]1C]

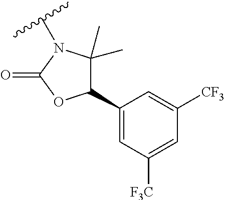

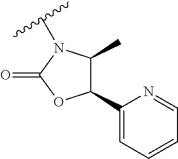 [SMILES: *N1C(=O)O[C@H](c2ccccn2)[C@@H]1C]

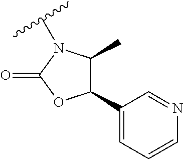 [SMILES: *N1C(=O)O[C@H](c2cccnc2)[C@@H]1C]

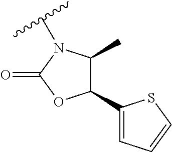 [SMILES: *N1C(=O)O[C@H](c2cccs2)[C@@H]1C]

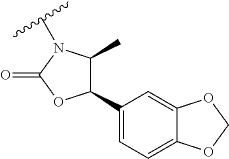 [SMILES: *N1C(=O)O[C@H](c2ccc3c(c2)OCO3)[C@@H]1C]

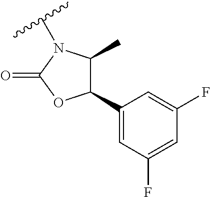 [SMILES: *N1C(=O)O[C@H](c2cc(F)cc(F)c2)[C@@H]1C]

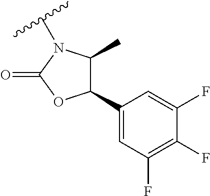 [SMILES: *N1C(=O)O[C@H](c2cc(F)c(F)c(F)c2)[C@@H]1C]

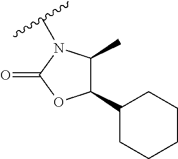 [SMILES: *N1C(=O)O[C@H](C2CCCCC2)[C@@H]1C]

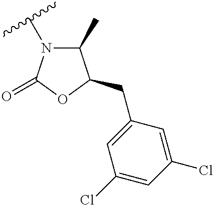 [SMILES: *N1C(=O)O[C@H](Cc2cc(Cl)cc(Cl)c2)[C@@H]1C]

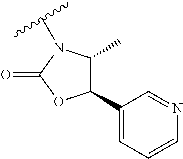 [SMILES: *N1C(=O)O[C@H](c2cccnc2)[C@H]1C]

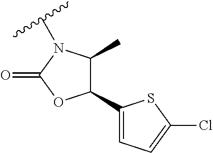 [SMILES: *N1C(=O)O[C@H](c2ccc(Cl)s2)[C@@H]1C]

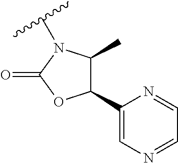 [SMILES: *N1C(=O)O[C@H](c2cnccn2)[C@@H]1C]

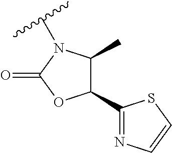 [SMILES: *N1C(=O)O[C@H](c2nccs2)[C@@H]1C]

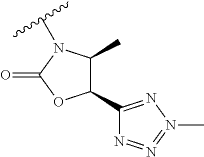

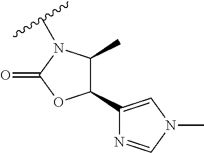 [SMILES: *N1C(=O)O[C@H](c2cn(C)cn2)[C@@H]1C]

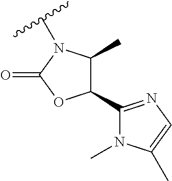 [SMILES: *N1C(=O)O[C@H](c2ncc(C)n2C)[C@@H]1C]

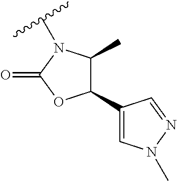 [SMILES: *N1C(=O)O[C@H](c2cnn(C)c2)[C@@H]1C]

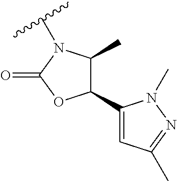 [SMILES: *N1C(=O)O[C@H](c2cc(C)nn2C)[C@@H]1C]

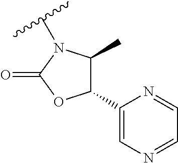 [SMILES: *N1C(=O)O[C@@H](c2cnccn2)[C@@H]1C]

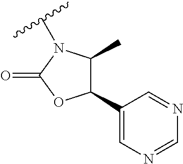 [SMILES: *N1C(=O)O[C@H](c2cncnc2)[C@@H]1C]

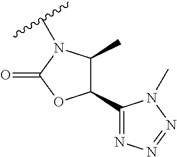 [SMILES: *N1C(=O)O[C@H](c2nnnn2C)[C@@H]1C]

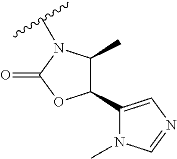 [SMILES: *N1C(=O)O[C@H](c2cncn2C)[C@@H]1C]

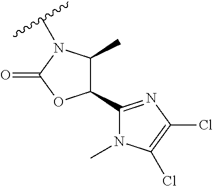 [SMILES: *N1C(=O)O[C@H](c2nc(Cl)c(Cl)n2C)[C@@H]1C]

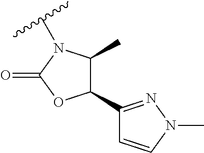 [SMILES: *N1C(=O)O[C@H](c2ccn(C)n2)[C@@H]1C]

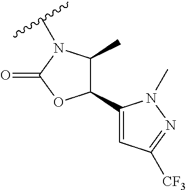 [SMILES: *N1C(=O)O[C@H](c2cc(C(F)(F)F)nn2C)[C@@H]1C]

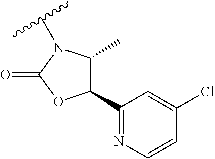 [SMILES: *N1C(=O)O[C@H](c2cc(Cl)ccn2)[C@H]1C]

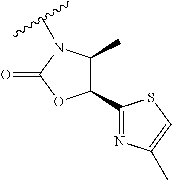 [SMILES: *N1C(=O)O[C@H](c2nc(C)cs2)[C@@H]1C]

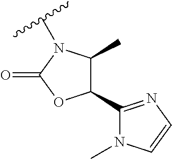 [SMILES: *N1C(=O)O[C@H](c2nccn2C)[C@@H]1C]

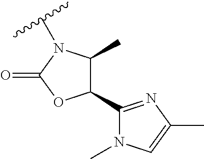 [SMILES: *N1C(=O)O[C@H](c2nc(C)cn2C)[C@@H]1C]

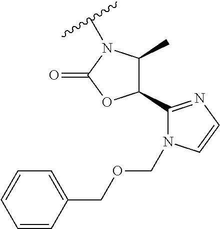 [SMILES: *N1C(=O)O[C@H](c2nccn2COCc2ccccc2)[C@@H]1C]

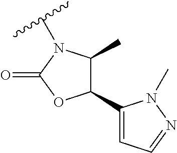 [SMILES: *N1C(=O)O[C@H](c2ccnn2C)[C@@H]1C]

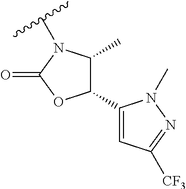 [SMILES: *N1C(=O)O[C@@H](c2cc(C(F)(F)F)nn2C)[C@H]1C]

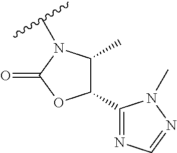 [SMILES: *N1C(=O)O[C@@H](c2ncnn2C)[C@H]1C]

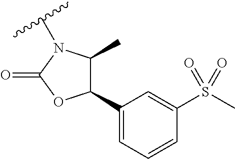 [SMILES: *N1C(=O)O[C@H](c2cccc(S(C)(=O)=O)c2)[C@@H]1C]

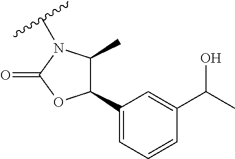 [SMILES: *N1C(=O)O[C@H](c2cccc(C(C)O)c2)[C@@H]1C]

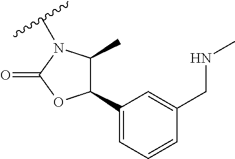 [SMILES: *N1C(=O)O[C@H](c2cccc(CNC)c2)[C@@H]1C]

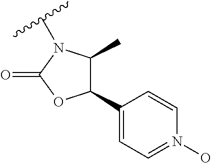

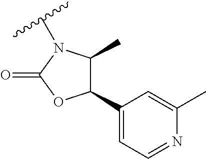 [SMILES: *N1C(=O)O[C@H](c2ccnc(C)c2)[C@@H]1C]

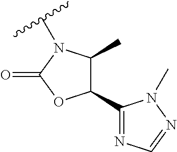 [SMILES: *N1C(=O)O[C@H](c2ncnn2C)[C@@H]1C]

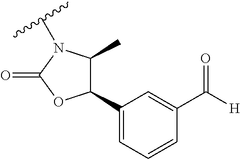 [SMILES: *N1C(=O)O[C@H](c2cccc(C=O)c2)[C@@H]1C]

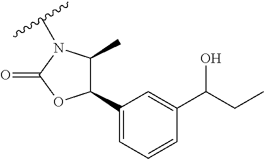 [SMILES: *N1C(=O)O[C@H](c2cccc(C(O)CC)c2)[C@@H]1C]

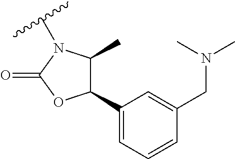 [SMILES: *N1C(=O)O[C@H](c2cccc(CN(C)C)c2)[C@@H]1C]

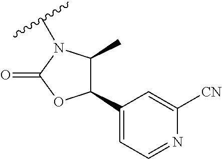 [SMILES: *N1C(=O)O[C@H](c2ccnc(C#N)c2)[C@@H]1C]

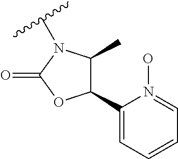

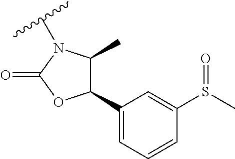 [SMILES: *N1C(=O)O[C@H](c2cccc(S(C)=O)c2)[C@@H]1C]

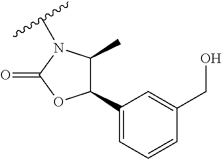 [SMILES: *N1C(=O)O[C@H](c2cccc(CO)c2)[C@@H]1C]

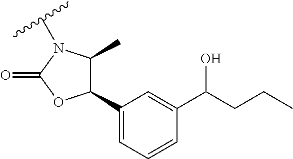 [SMILES: *N1C(=O)O[C@H](c2cccc(C(O)CCC)c2)[C@@H]1C]

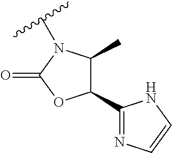 [SMILES: *N1C(=O)O[C@H](c2ncc[nH]2)[C@@H]1C]

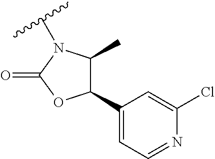 [SMILES: *N1C(=O)O[C@H](c2ccnc(Cl)c2)[C@@H]1C]

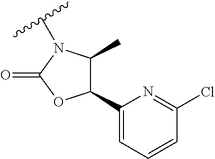 [SMILES: *N1C(=O)O[C@H](c2cccc(Cl)n2)[C@@H]1C]

and pharmaceutically acceptable salts of the foregoing;

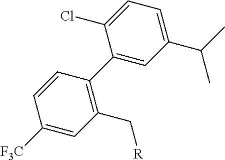

R

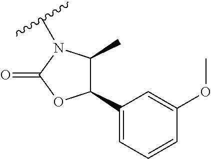 [SMILES: *N1C(=O)O[C@H](c2cccc(OC)c2)[C@@H]1C]

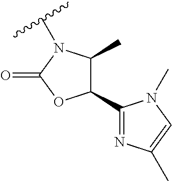 [SMILES: *N1C(=O)O[C@H](c2nc(C)cn2C)[C@@H]1C]

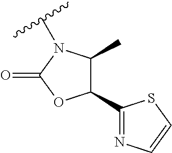 [SMILES: *N1C(=O)O[C@H](c2nccs2)[C@@H]1C]

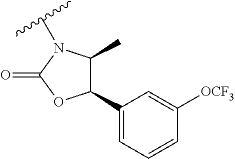 [SMILES: *N1C(=O)O[C@H](c2cccc(OC(F)(F)F)c2)[C@@H]1C]

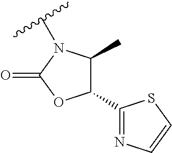 [SMILES: *N1C(=O)O[C@@H](c2nccs2)[C@@H]1C]

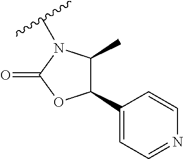 [SMILES: *N1C(=O)O[C@H](c2ccncc2)[C@@H]1C]

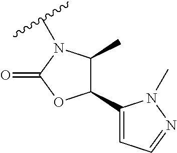 [SMILES: *N1C(=O)O[C@H](c2ccnn2C)[C@@H]1C]

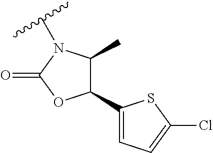 [SMILES: *N1C(=O)O[C@H](c2ccc(Cl)s2)[C@@H]1C]

and pharmaceutically acceptable salts of the foregoing;

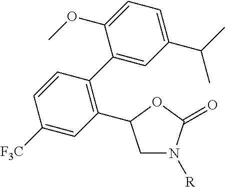

R

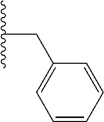 [SMILES: *Cc1ccccc1]

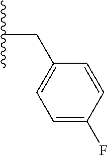 [SMILES: *Cc1ccc(F)cc1]

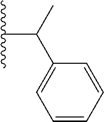 [SMILES: *C(C)c1ccccc1]

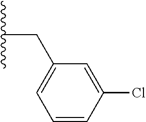 [SMILES: *Cc1cccc(Cl)c1]

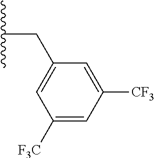 [SMILES: *Cc1cc(C(F)(F)F)cc(C(F)(F)F)c1]

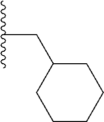 [SMILES: *CC1CCCCC1]

and pharmaceutically acceptable salts of the foregoing;

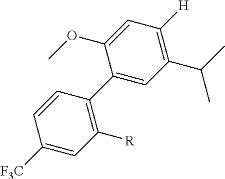

R

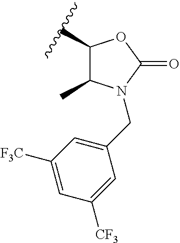 [SMILES: *[C@H]1OC(=O)N(Cc2cc(C(F)(F)F)cc(C(F)(F)F)c2)[C@H]1C]

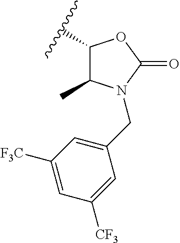 [SMILES: *[C@@H]1OC(=O)N(Cc2cc(C(F)(F)F)cc(C(F)(F)F)c2)[C@H]1C]

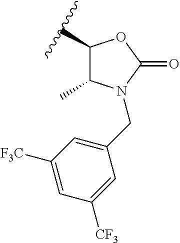 [SMILES: *[C@H]1OC(=O)N(Cc2cc(C(F)(F)F)cc(C(F)(F)F)c2)[C@@H]1C]

and pharmaceutically acceptable salts of the foregoing;

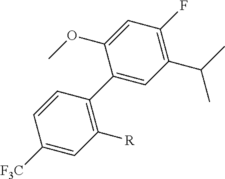

R

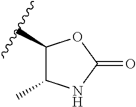 [SMILES: *[C@H]1OC(=O)N[C@@H]1C]

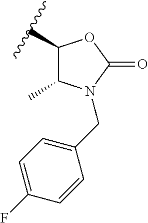 [SMILES: *[C@H]1OC(=O)N(Cc2ccc(F)cc2)[C@@H]1C]

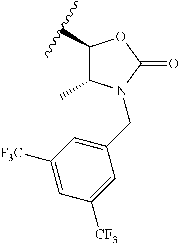 [SMILES: *[C@H]1OC(=O)N(Cc2cc(C(F)(F)F)cc(C(F)(F)F)c2)[C@@H]1C]

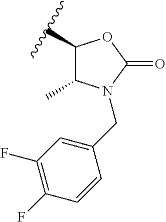 [SMILES: *[C@H]1OC(=O)N(Cc2ccc(F)c(F)c2)[C@@H]1C]

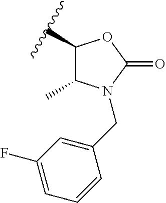 [SMILES: *[C@H]1OC(=O)N(Cc2cccc(F)c2)[C@@H]1C]

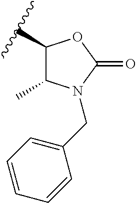 [SMILES: *[C@H]1OC(=O)N(Cc2ccccc2)[C@@H]1C]

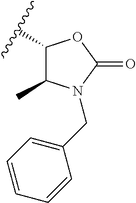 [SMILES: *[C@@H]1OC(=O)N(Cc2ccccc2)[C@H]1C]

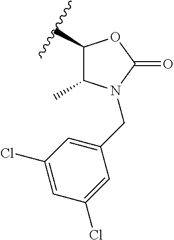 [SMILES: *[C@H]1OC(=O)N(Cc2cc(Cl)cc(Cl)c2)[C@@H]1C]

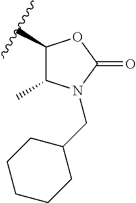 [SMILES: *[C@H]1OC(=O)N(CC2CCCCC2)[C@@H]1C]

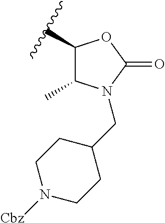 [SMILES: *[C@H]1OC(=O)N(CC2CCN(C(=O)OCc3ccccc3)CC2)[C@@H]1C]

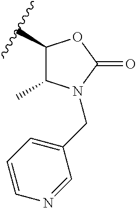 [SMILES: *[C@H]1OC(=O)N(Cc2cccnc2)[C@@H]1C]

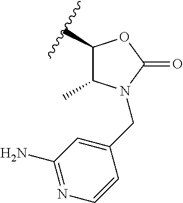 [SMILES: *[C@H]1OC(=O)N(Cc2ccnc(N)c2)[C@@H]1C]

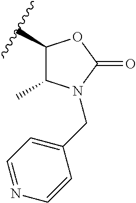 [SMILES: *[C@H]1OC(=O)N(Cc2ccncc2)[C@@H]1C]

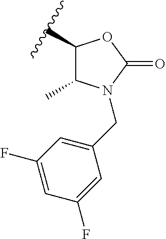 [SMILES: *[C@H]1OC(=O)N(Cc2cc(F)cc(F)c2)[C@@H]1C]

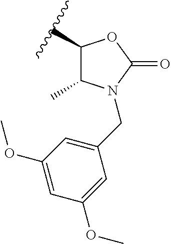 [SMILES: *[C@H]1OC(=O)N(Cc2cc(OC)cc(OC)c2)[C@@H]1C]

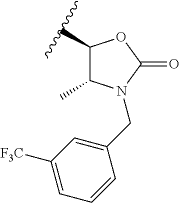 [SMILES: *[C@H]1OC(=O)N(Cc2cccc(C(F)(F)F)c2)[C@@H]1C]

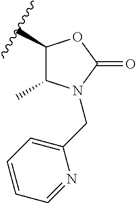 [SMILES: *[C@H]1OC(=O)N(Cc2ccccn2)[C@@H]1C]

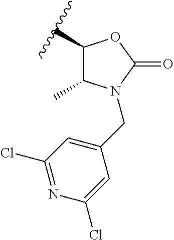 [SMILES: *[C@H]1OC(=O)N(Cc2cc(Cl)nc(Cl)c2)[C@@H]1C]

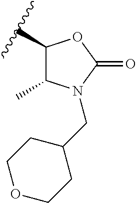 [SMILES: *[C@H]1OC(=O)N(CC2CCOCC2)[C@@H]1C]

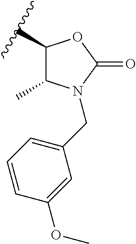 [SMILES: *[C@H]1OC(=O)N(Cc2cccc(OC)c2)[C@@H]1C]

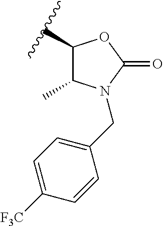 [SMILES: *[C@H]1OC(=O)N(Cc2ccc(C(F)(F)F)cc2)[C@@H]1C]

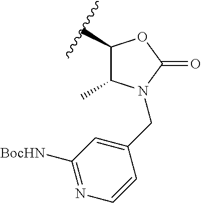 [SMILES: *[C@H]1OC(=O)N(Cc2ccnc(NC(=O)OC(C)(C)C)c2)[C@@H]1C]

and pharmaceutically acceptable salts of the foregoing; and

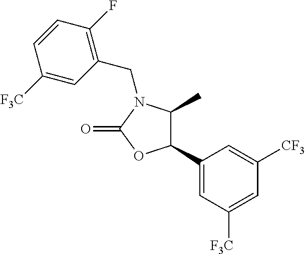 [SMILES: C[C@H]1[C@@H](c2cc(C(F)(F)F)cc(C(F)(F)F)c2)OC(=O)N1Cc1cc(C(F)(F)F)ccc1F]

R

| | |
|---|---|
| 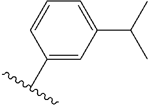 [SMILES: *c1cccc(C(C)C)c1] | 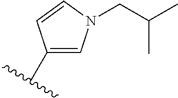 [SMILES: *c1ccn(CC(C)C)c1] |
| 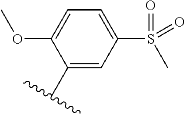 [SMILES: *c1cc(S(C)(=O)=O)ccc1OC] | 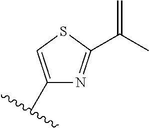 [SMILES: *c1csc(C(=C)C)n1] |
| 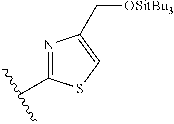 | 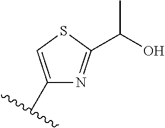 [SMILES: *c1csc(C(C)O)n1] |
| 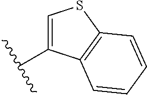 [SMILES: *c1csc2ccccc12] | 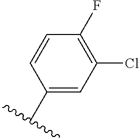 [SMILES: *c1ccc(F)c(Cl)c1] |
| 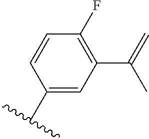 [SMILES: *c1ccc(F)c(C(=C)C)c1] | 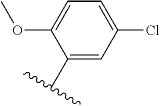 [SMILES: *c1cc(Cl)ccc1OC] |
| 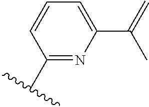 [SMILES: *c1cccc(C(=C)C)n1] | 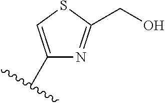 [SMILES: *c1csc(CO)n1] |
| 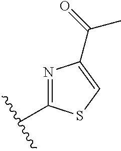 [SMILES: *c1nc(C(C)=O)cs1] | 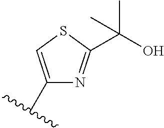 [SMILES: *c1csc(C(C)(C)O)n1] |
| 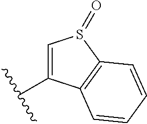 [SMILES: *C1=CS(=O)c2ccccc21] | 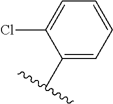 [SMILES: *c1ccccc1Cl] |
| 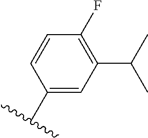 [SMILES: *c1ccc(F)c(C(C)C)c1] | 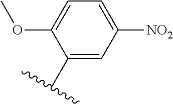 [SMILES: *c1cc([N+](=O)[O-])ccc1OC] |
| 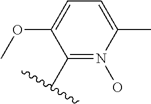 | 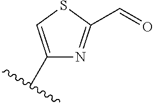 [SMILES: *c1csc(C=O)n1] |
| 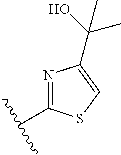 [SMILES: *c1nc(C(C)(C)O)cs1] | 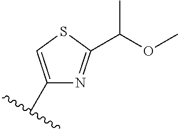 [SMILES: *c1csc(C(C)OC)n1] |
| 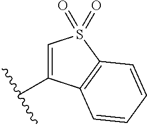 [SMILES: *C1=CS(=O)(=O)c2ccccc21] | 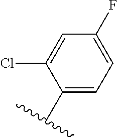 [SMILES: *c1ccc(F)cc1Cl] |
| 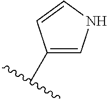 [SMILES: *c1cc[nH]c1] | 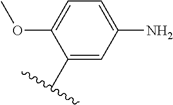 [SMILES: *c1cc(N)ccc1OC] |
| 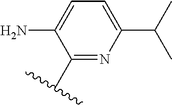 [SMILES: *c1nc(C(C)C)ccc1N] | 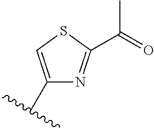 [SMILES: *c1csc(C(C)=O)n1] |
| 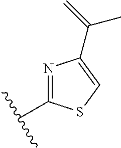 [SMILES: *c1nc(C(=C)C)cs1] | 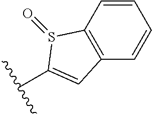 [SMILES: *C1=Cc2ccccc2S1=O] |
| 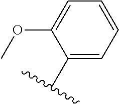 [SMILES: *c1ccccc1OC] | 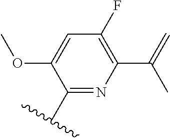 [SMILES: *c1nc(C(=C)C)c(F)cc1OC] |
| 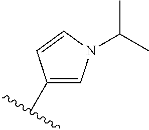 [SMILES: *c1ccn(C(C)C)c1] | 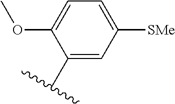 [SMILES: *c1cc(SC)ccc1OC] |
| 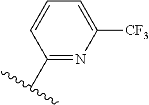 [SMILES: *c1cccc(C(F)(F)F)n1] | 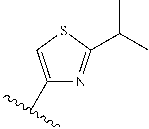 [SMILES: *c1csc(C(C)C)n1] |
| 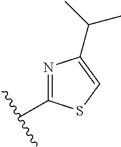 [SMILES: *c1nc(C(C)C)cs1] | 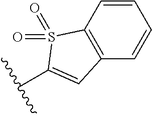 [SMILES: *C1=Cc2ccccc2S1(=O)=O] |
| 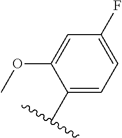 [SMILES: *c1ccc(F)cc1OC] | 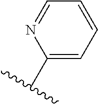 [SMILES: *c1ccccn1] |
| 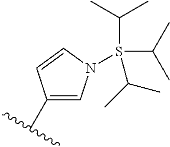 [SMILES: *c1ccn(S(C(C)C)(C(C)C)C(C)C)c1] | 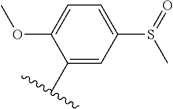 [SMILES: *c1cc(S(C)=O)ccc1OC] |
| 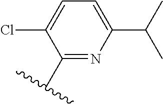 [SMILES: *c1nc(C(C)C)ccc1Cl] | 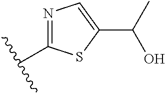 [SMILES: *c1ncc(C(C)O)s1] |
| 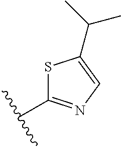 [SMILES: *c1ncc(C(C)C)s1] | 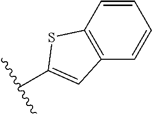 [SMILES: *c1cc2ccccc2s1] |
| 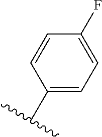 [SMILES: *c1ccc(F)cc1] | 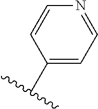 [SMILES: *c1ccncc1] |
| 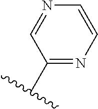 [SMILES: *c1cnccn1] | 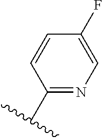 [SMILES: *c1ccc(F)cn1] |
| 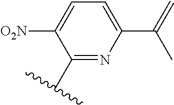 [SMILES: *c1nc(C(=C)C)ccc1[N+](=O)[O-]] | 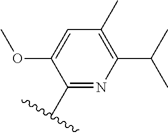 [SMILES: *c1nc(C(C)C)c(C)cc1OC] |
| 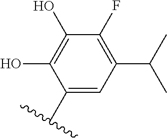 [SMILES: *c1cc(C(C)C)c(F)c(O)c1O] | 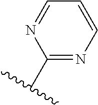 [SMILES: *c1ncccn1] |
| 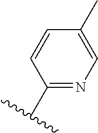 [SMILES: *c1ccc(C)cn1] | 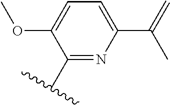 [SMILES: *c1nc(C(=C)C)ccc1OC] |
| 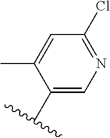 [SMILES: *c1cnc(Cl)cc1C] | 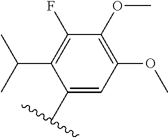 [SMILES: *c1cc(OC)c(OC)c(F)c1C(C)C] |
| 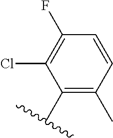 [SMILES: *c1c(C)ccc(F)c1Cl] | 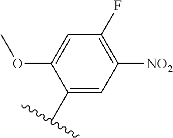 [SMILES: *c1cc([N+](=O)[O-])c(F)cc1OC] |
| 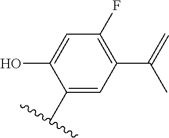 [SMILES: *c1cc(C(=C)C)c(F)cc1O] | 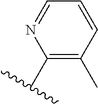 [SMILES: *c1ncccc1C] |
| 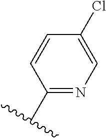 [SMILES: *c1ccc(Cl)cn1] | 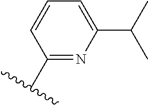 [SMILES: *c1cccc(C(C)C)n1] |
| 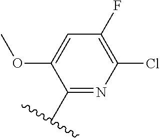 [SMILES: *c1nc(Cl)c(F)cc1OC] | 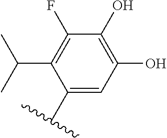 [SMILES: *c1cc(O)c(O)c(F)c1C(C)C] |
| 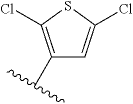 [SMILES: *c1cc(Cl)sc1Cl] | 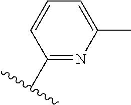 [SMILES: *c1cccc(C)n1] |
| 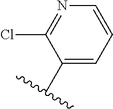 [SMILES: *c1cccnc1Cl] | 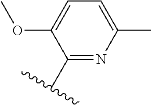 |
| 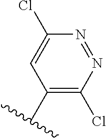 [SMILES: *c1cc(Cl)nnc1Cl] | 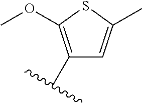 |
| 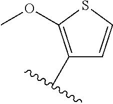 [SMILES: *c1ccsc1OC] | 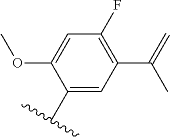 [SMILES: *c1cc(C(=C)C)c(F)cc1OC] |
| 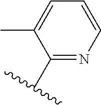 [SMILES: *c1ncccc1C] | 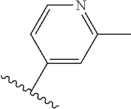 [SMILES: *c1ccnc(C)c1] |
| 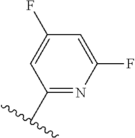 [SMILES: *c1cc(F)cc(F)n1] | 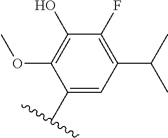 [SMILES: *c1cc(C(C)C)c(F)c(O)c1OC] |
| 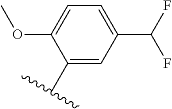 [SMILES: *c1cc(C(F)F)ccc1OC] | 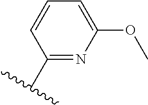 [SMILES: *c1cccc(OC)n1] |
| 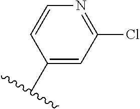 [SMILES: *c1ccnc(Cl)c1] | 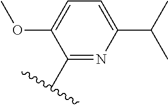 [SMILES: *c1nc(C(C)C)ccc1OC] |
| 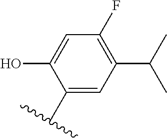 [SMILES: *c1cc(C(C)C)c(F)cc1O] | 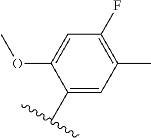 |
| 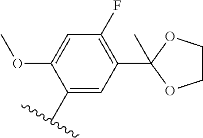 [SMILES: *c1cc(C2(C)OCCO2)c(F)cc1OC] | 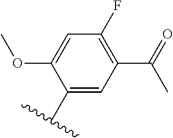 [SMILES: *c1cc(C(C)=O)c(F)cc1OC] |

and pharmaceutically acceptable salts of the foregoing.

Additional CETP inhibitors useful in the compositions and methods of the invention are disclosed in US20150374675 A1 and include, but are not limited to:
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S-[4,5-dichloro-2-(1-isopentylcyclopentanecarbonylamino)-phenyl]2,2-dimethylthiopropionate;
5-[2-(1-isopentylcyclohexanecarbonylamino)-4-trifluoromethylphenyl]2,2-dimethylthiopropionate;
O-methyl S-[2-(1-isopentylcyclohexanecarbonylamino phenyl monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate;
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)-phenyl]2,2-dimethylthiopropionate;
S-[4,5-dichloro-2-(1-cyclopropylmethylcyclohexanecarbonylamino)phenyl]2,2-dimethylthiopropionate;
S-[4,5-dichloro-2-(1-cyclohexylmethylcyclohexanecarbonylamino)phenyl]2,2-dimethylthiopropionate;
S-[4,5-dichloro-2-(1-isopropylcyclohexanecarbonylamino)-phenyl]2,2-dimethylthiopropionate;
S-[4,5-dichloro-2-(1-isopentylcycloheptanecarbonylamino)-phenyl]2,2-dimethylthiopropionate;
S-[4,5-dichloro-2-(1-isopentylcyclobutanecarbonylamino)-phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate; bis-[4,5-dichloro-2-(1-isopentylcyclohexanecarbonylamino)-phenyl]disulfide;
2-tetrahydrofurylmethyl 2-(1-isopentylcyclohexanecarbonylamino)phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate; bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-(2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; and
S-[2-(1-isobutylcyclohexanecarbonylamino)phenyl]-1-acetyl-piperidine-4-thiocarboxylate; and
pharmaceutically acceptable salts of the foregoing.

Additional examples of CETP inhibitors useful in the compositions and methods of the invention include, but are not limited to: torcetrapib; dalcetrapib; anacetrapib; evacetrapib; obicetrapib; BMS-79531; CP-800,569; DRL-17822; JNJ-28545595; JNJ-28614872; BAY 19-4789; BAY 38-1315; 1,1,1-trifluoro-3-((3-phenoxyphenyl)(3-(1,1,2,2-tetrafluoroethoxy)benzyl)amino)propan-2-ol; (R)-3-((4-(4-chloro-3-ethylphenoxy)pyrimidin-2-yl)(3-(1,1,2,2-tetrafluoroethoxy)benzyl)amino)-1,1,1-trifluoropropan-2-ol; (R)-3-((3-(4-chloro-3-ethylphenoxy)phenyl)(3-(1,1,2,2-tetrafluoroethoxy)benzyl)amino)-1,1,1-trifluoropropan-2-ol (CP-800,569); N-(4-(5,7-dimethylbenzo[d]oxazol-2-yl)phenyl)-2-(o-tolyloxy)acetamide; 2-(4-chloro-2,3-dimethylphenoxy)-N-(4-(5-cyanobenzo[d]oxazol-2-yl)phenyl)acetamide; N-(4-(5-chlorobenzo[d]oxazol-2-yl)phenyl)-2-(o-tolyloxy)acetamide; N-(4-(5-chlorobenzo[d]oxazol-2-yl)phenyl)-2-(o-tolyloxy)acetamide; N-(4-(5-cyano-7-methylbenzo[d]oxazol-2-yl)phenyl)-2-(o-tolyloxy)acetamide; N-(4-(5-cyano-7-(2-hydroxypropan-2-yl)benzo[d]oxazol-2-yl)phenyl)-2-(o-tolyloxy)acetamide; 2-(4-((2-(3,3,3-trifluoro-2-methyl-2-(trifluoromethyl)propoxy)ethyl)amino)phenyl)benzo[d]oxazole-5-carbonitrile; tert-butyl 4-(2-((4-(5-cyanobenzo[d]oxazol-2-yl)phenyl)amino)-2-oxoethoxy)piperidine-1-carboxylate; N-(4-(5-cyano-7-methylbenzo[d]oxazol-2-yl)phenyl)-2-(4-(3-(trifluoromethyl)phenyl)piperazin-1-yl)acetamide; N-(4-(5-cyano-7-methylbenzo[d]oxazol-2-yl)phenyl)-2-(4-(4-(trifluoromethyl)phenyl)piperazin-1-yl)acetamide; N-(4-(5-cyano-7-methylbenzo[d]oxazol-2-yl)phenyl)-2-(4-(5-(trifluoromethyl)pyridin-2-yl)piperazin-1-yl)acetamide; 4-(5-cyano-7-methylbenzo[d]oxazol-2-yl)-N-((1-(4-(trifluoromethyl)phenyl)piperidin-4-yl)methyl)benzamide; 4-(5-cyano-7-isopropylbenzo[d]oxazol-2-yl)-N-((1-(5-(trifluoromethyl)pyridin-2-yl)piperidin-4-yl)methyl)benzamide; 4-(5-cyano-7-isopropylbenzo[d]oxazol-2-yl)-N-((1-(5-phenylpyridin-2-yl)piperidin-4-yl)methyl)benzamide; 4-(5-cyano-7-isopropylbenzo[d]oxazol-2-yl)-N-((1-(5-(2-isopropyl-5-methylphenyl)pyridin-2-yl)piperidin-4-yl)methyl)benzamide; 4-(5-cyano-7-isopropylbenzo[d]oxazol-2-yl)-N-((1-(5-(5-fluoro-2-isopropylphenyl)pyridin-2-yl)piperidin-4-yl)methyl)benzamide; (R)-4-(5-cyano-7-isopropylbenzo[d]oxazol-2-yl)-N-((2-oxo-3-(5-(2-(trifluoromethoxy)phenyl)pyridin-2-yl)oxazolidin-5-yl)methyl)benzamide; (S)-4-(5-cyano-7-isopropylbenzo[d]oxazol-2-yl)-N-((2-oxo-3-(5-(2-(trifluoromethoxy)phenyl)pyridin-2-yl)oxazolidin-5-yl)methyl)benzamide; (R)-4-(5-cyano-7-isopropylbenzo[d]oxazol-2-yl)-N-((5-methyl-2-oxo-3-(5-(2-(trifluoromethoxy)phenyl)pyridin-2-yl)oxazolidin-5-yl)methyl)benzamide; (S))-4-(5-cyano-7-isopropylbenzo[d]oxazol-2-yl)-N-((5-methyl-2-oxo-3-(5-(2-trifluoromethoxy)phenyl)pyridin-2-yl)oxazolidin-5-yl)methyl)benzamide; N-((4-(4-(tert-butyl)phenyl)cyclohexyl)methyl)-4-(5-cyano-7-isopropylbenzo[d]oxazol-2-yl)benzamide; methyl (3,5-bis(trifluoromethyl)benzyl)((5′-isopropyl-2′-methoxy-4-(trifluoromethyl)-[1,1′-biphenyl]-2-yl)methyl)carbamate; methyl (3,5-bis(trifluoromethyl)benzyl)(2-((ethoxycarbonyl)(propyl)amino)-5-(trifluoromethyl)benzyl)carbamate; methyl (3,5-bis(trifluoromethyl)benzyl)(2-(2-oxooxazolidin-3-yl)-5-(trifluoromethyl)benzyl)carbamate; methyl (3,5-bis(trifluoromethyl)benzyl)(2-(2-oxoimidazolidin-1-yl)-5-(trifluoromethyl)benzyl)carbamate; 4-(3,5-bis(trifluoromethyl)phenyl)-3-((5′-isopropyl-2′-methoxy-4-(trifluoromethyl)-[1,1′-biphenyl]-2-yl)methyl)oxazolidin-2-one; (R)-4-(3,5-bis(trifluoromethyl)phenyl)-3-((5′-isopropyl-2′-methoxy-4-(trifluoromethyl)-[1,1′-biphenyl]-2-yl)methyl)oxazolidin-2-one; (S)-4-(3,5-bis(trifluoromethyl)phenyl)-3-((5′-isopropyl-2′-methoxy-4-(trifluoromethyl)-[1,1′-biphenyl]-2-yl)methyl)oxazolidin-2-one; (4R,5S)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; (4S,5R)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; (4R,5R)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; (4S,5S)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; 5-(2,6-bis(trifluoromethyl)pyridin-4-yl)-3-((4′-fluoro-5′-isopropyl-2′-methoxy-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-2′-hydroxy-5′-isopropyl-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-2′,3′-dihydroxy-5′-isopropyl-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-2′,3′-dihydroxy-5′-(2-hydroxypropan-2-yl)-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-5′-isopropyl-2′-methoxy-4-(trifluoromethyl)-3,4,5,6-tetrahydro-[1,1′-biphenyl]-2-yl)methyl)-4-methyloxazolidin-2-one; N-(6′-(((4S,5S)-5-(3,5-bis(trifluoromethyl)phenyl)-4-methyl-2-oxooxazolidin-3-yl)methyl)-2-methoxy-4′,4′-dimethyl-2′,3′,4′,5′-tetrahydro-[1,1′-biphenyl]-4-yl)-N-methylacetamide; (S)-5-(3,5-bis(trifluoromethyl)phenyl)-3-((4′-fluoro-5′-isopropyl-2′-methoxy-4-(trifluoromethyl)-[1,1′-biphenyl]-2-yl)methyl)-4,4-dimethyloxazolidin-2-one; 3-(6′-(((4S,5S)-5-(3,5-bis(trifluoromethyl)phenyl)-4-methyl-2-oxooxazolidin-3-yl)methyl)-2-methoxy-4′,4′-dimethyl-2′,3′,4′,5′-tetrahydro-[1,1′-biphenyl]-4-yl)-2,2-dimethylpropanoic acid; 3-(3-(2-(((4S,5S)-5-(3,5-bis(trifluoromethyl)phenyl)-4-methyl-2-oxooxazolidin-3-yl)methyl)-6-methoxypyridin-3-yl)-4-methoxyphenyl)propanoic acid; 3′-(6-(azetidin-1-yl)-2-(((4S,5S)-5-(3,5-bis(trifluoromethyl)phenyl)-4-methyl-2-oxooxazolidin-3-yl)methyl)pyridin-3-yl)-5′-fluoro-4′-methoxy-2-methyl-[1,1′-biphenyl]-4-carboxylic acid; isopropyl (2R,4S)-4-((3,5-bis(trifluoromethyl)benzyl)(2H-tetrazol-5-yl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-methyl-2H-tetrazol-5-yl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-(2-cyanoethyl)-2H-tetrazol-5-yl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-(2-hydroxyethyl)-2H-tetrazol-5-yl)amino)-2-ethyl-6-(trifluoromethyl)-3,4-dihydroquinoline-1(2H)-carboxylate; isopropyl (2R,4S)-4-((2-(2-aminoethyl)-2H-tetrazol-5-yl)(3,5-bis(trifluoromethyl)benzyl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-(2-hydroxypropyl)-2H-tetrazol-5-yl)amino)-2-ethyl-6-(trifluoromethyl)-3,4-dihydroquinoline-1(2H)-carboxylate; ethyl (2R,4S)-4-((3,5-bis(trifluoromethyl)benzyl)(2-methyl-2H-tetrazol-5-yl)amino)-2-ethyl-6-(trifluoromethyl)-3,4-dihydroquinoline-1(2H)-carboxylate; ethyl (2R,4S)-4-((3,5-bis(trifluoromethyl)benzyl)(2-methyl-2H-tetrazol-5-yl)amino)-2-ethyl-8-methyl-6-(trifluoromethyl)-3,4-dihydroquinoline-1(2H)-carboxylate; ethyl (2R,4S)-4-(N-(3,5-bis(trifluoromethyl)benzyl)acetamido)-2-ethyl-6-(trifluoromethyl)-3,4-dihydro-1,5-naphthyridine-1(2H)-carboxylate; ethyl (2R,4S)-4-(N-(3,5-bis(trifluoromethyl)benzyl)acetamido)-2-ethyl-6-methoxy-3,4-dihydro-1,5-naphthyridine-1(2H)-carboxylate; ethyl (2R,4S)-4-(N-(3,5-bis(trifluoromethyl)benzyl)acetamido)-6-(dimethylamino)-2-ethyl-3,4-dihydro-1,5-naphthyridine-1(2H)-carboxylate; ethyl (2R,4S)-4-((3,5-bis(trifluoromethyl)benzyl)(2-methyl-2H-tetrazol-5-yl)amino)-2-ethyl-6-(trifluoromethyl)-3,4-dihydro-1,5-naphthyridine-1(2H)-carboxylate; ethyl (2R,4S)-4-((3,5-bis(trifluoromethyl)benzyl)(2-methyl-2H-tetrazol-5-yl)amino)-2-ethyl-6-methoxy-3,4-dihydro-1,5-naphthyridine-1(2H)-carboxylate; ethyl (2R,4S)-4-((3,5-bis(trifluoromethyl)benzyl)(2-methyl-2H-tetrazol-5-yl)amino)-6-(dimethylamino)-2-ethyl-3,4-dihydro-1,5-naphthyridine-1(2H)-carboxylate; isopropyl (2R,4S)-4-((3,5-bis(trifluoromethyl)benzyl)(2-methyl-2H-tetrazol-5-yl)amino)-2-ethyl-6-(trifluoromethyl)-3,4-dihydro-1,5-naphthyridine-1(2H)-carboxylate; isopropyl (2R,4S)-4-((3-chloro-5-(trifluoromethyl)benzyl)(2-methyl-2H-tetrazol-5-yl)amino)-2-ethyl-6-(trifluoromethyl)-3,4-dihydro-1,5-naphthyridine-1(2H)-carboxylate; isopropyl (2R,4S)-4-((3,5-dichlorobenzyl)(2-methyl-2H-tetrazol-5-yl)amino)-2-ethyl-6-methyl-3,4-dihydro-1,5-naphthyridine-1(2H)-carboxylate; 5-(((3,5-bis(trifluoromethyl)benzyl)(2-methyl-2H-tetrazol-5-yl)amino)methyl)-N-(cyclopentylmethyl)-N-ethyl-1,3-dimethyl-1H-pyrazolo[3,4-b]pyridin-6-amine; 6-(((2-(bis(cyclopropylmethyl)amino)-7,7-dimethyl-6,7-dihydro-5H-cyclopenta[b]pyridin-3-yl)methyl)(3,5-bis(trifluoromethyl)benzyl)amino)benzo[d]oxazol-2(3H)-one; 3#(3,5-bis(trifluoromethyl)benzyl)(5-morpholinopyrimidin-2-yl)amino)methyl)-N,N-bis(cyclopropylmethyl)-7,7-dimethyl-6,7-dihydro-5H-cyclopenta[b]pyridin-2-amine; isopropyl (2R)-4-((3,5-bi s(trifluoromethyl)benzyl)(5-(1-methyl-1H-pyrazol-4-yl)pyrimidin-2-yl)amino)-2-ethylpyrrolidine-1-carboxylate; 3-(((3,5-bis(trifluoromethyl)benzyl)(2-methyl-2H-tetrazol-5-yl)amino)methyl)-5-bromo-N-(cyclopentylmethyl)-N-ethyl-6-methylpyridin-2-amine; 3-(((3,5-bis(trifluoromethyl)benzyl)(2-methyl-2H-tetrazol-5-yl)amino)methyl)-N-(cyclopentylmethyl)-N-ethyl-6-methyl-5-(methylthio)pyridin-2-amine; ((2R)-4-((3,5-bis(trifluoromethyl)benzyl)(5-(1-methyl-1H-pyrazol-4-yl)pyrimidin-2-yl)amino)-2-ethylpyrrolidin-1-yl)(cyclohexyl)methanone; (1r,4r)-4-(((2-(((3,5-bis(trifluoromethyl)benzyl)(2-methyl-2H-tetrazol-5-yl)amino)methyl)-4-(trifluoromethyl)phenyl)(ethyl)amino)methyl)cyclohexane-1-carboxylic acid; 3-((((3-((cyclopentylmethyl)(ethyl)amino)-5,6,7,8-tetrahydronaphthalen-2-yl)methyl)(2-methyl-2H-tetrazol-5-yl)amino)methyl)-5-(trifluoromethyl)benzonitrile; (1R,4r)-4-(((2R,6S)-44(3,5-bis(trifluoromethyl)benzyl)(5-(1-methyl-1H-pyrazol-4-yl)pyrimidin-2-yl)amino)-2,6-diethylpiperidine-1-carbonyl)oxy)cyclohexane-1-carboxylic acid; (1R,3R)-3-(((2R,6S)-4-((3,5-bis(trifluoromethyl)benzyl)(5-(1-methyl-1H-pyrazol-4-yl)pyrimidin-2-yl)amino)-2,6-diethylpiperidine-1-carbonyl)oxy)cyclobutane-1-carboxylic acid; 1-(2-((3,5-bis(trifluoromethyl)benzyl)(2-(ethyl(2-methoxyethyl)amino)benzyl)amino)pyrimidin-5-yl)piperidine-4-carboxylic acid; 5(((1-(3,5-bis(trifluoromethyl)phenyl)ethyl)(5-(2-(methylsulfonyl)ethoxy)pyrimidin-2-yl)amino)methyl)-N-(cyclopentylmethyl)-N-ethyl-1,3-dimethyl-1H-indazol-6-amine; N-(1-(3,5-bis(trifluoromethyl)phenyl)ethyl)-N-(2-((cyclopentylmethyl)(ethyl)amino)-5-(trifluoromethyl)benzyl)-5-(2-(methylsulfonyl)ethoxy)pyrimidin-2-amine; 442-((3,5-bis(trifluoromethyl)benzyl)((3-((cyclopropylmethyl)(propyl)amino)quinolin-2-yl)methyl)amino)pyrimidin-5-yl)oxy)butanoic acid; 3-((((3-((cyclopentylmethyl)(ethyl)amino)-6-methoxypyridin-2-yl)methyl)(5-(2-(methylsulfonyl)ethoxy)pyrimidin-2-yl)amino)methyl)-5-(trifluoromethyl)benzonitrile; 2-((1S,4r)-4-(((2-((((S)-1-(3,5-bis(trifluoromethyl)phenyl)ethyl)(5-(2-(methylsulfonyl)ethoxy)pyrimidin-2-yl)amino)methyl)-4-(trifluoromethyl)phenyl)(ethyl)amino)methyl)cyclohexyl)acetic acid; ethyl (2R,4S)-4-((3,5-bis(trifluoromethyl)benzyl)(5-(2-(methylsulfonyl)ethoxy)pyrimidin-2-yl)amino)-2-ethyl-6-methoxy-3,4-dihydro-1,5-naphthyridine-1(2H)-carboxylate; ethyl (2R,4S)-4-((3,5-bis(trifluoromethyl)benzyl)(5-morpholinopyrimidin-2-yl)amino)-2-ethyl-6-(trifluoromethyl)-3,4-dihydroquinoline-1(2H)-carboxylate; ethyl (2R,4S)-4-((3,5-bis(trifluoromethyl)benzyl)(5-morpholinopyrimidin-2-yl)amino)-2-ethyl-6-methoxy-3,4-dihydro-1,5-naphthyridine-1(2H)-carboxylate; isopropyl 5-((3,5-bis(trifluoromethyl)benzyl)(2-methyl-2H-tetrazol-5-yl)amino)-7-methyl-8-(trifluoromethyl)-2,3,4,5-tetrahydro-1H-benzo[b]azepine-1-carboxylate; isopropyl 5-(N-(3,5-bis(trifluoromethyl)benzyl)acetamido)-7-methyl-2,3,4,5-tetrahydro-1H-benzo[b]azepine-1-carboxylate; 3-(5-(4-chloro-3-ethylphenoxy)-2-(3-(1,1,2,2-tetrafluoroethoxy)phenyl)-3,4-dihydroquinolin-1(2H)-yl)-1,1,1-trifluoropropan-2-ol; (S)-1,1,1-trifluoro-34R)-2-(3-(1,1,2,2-tetrafluoroethoxy)phenyl)-5-(4-(trifluoromethoxy)phenyl)-3,4-dihydroquinolin-1(2H)-yl)propan-2-ol (JNJ-28545595); (S)-1,1,1-trifluoro-3-((S)-3-(3-(1,1,2,2-tetrafluoroethoxy)phenyl)-8-(4-(trifluoromethoxy)phenyl)-2,3-dihydro-4H-benzo[b][1,4]oxazin-4-yl)propan-2-ol (JNJ-28614872); (R)-3-((R)-4-(3-(difluoromethoxy)benzyl)-2-(3-(trifluoromethyl)phenyl)-3,4-dihydroquinoxalin-1(2H)-yl)-1,1,1-trifluoropropan-2-ol; (S)-(2-cyclopentyl-4-ethyl-5-hydroxy-7,7-dimethyl-5,6,7, 8-tetrahydroquinolin-3-yl)(4-(trifluoromethyl)phenyl)methanone; (S)-2-cyclopentyl-3-((S)-fluoro(4-(trifluoromethyl)phenyl)methyl)-4-(4-fluorophenyl)-7,7-dimethyl-5,6,7,8-tetrahydroquinolin-5-ol (BAY 19-4789); (S)-3′-((S)-fluoro(4-(trifluoromethyl)phenyl)methyl)-4′-(4-fluorophenyl)-2′-isopropyl-5′,8′-dihydro-6′H-spiro[cyclobutane-1,7′-quinolin]-5′-ol (BAY 38-1315); (S)-4-cyclohexyl-2-cyclopentyl-3-((S)-hydroxy(4-(trifluoromethyl)phenyl)methyl)-7,7-dimethyl-5,6,7,8-tetrahydroquinolin-5-ol; (S)-4-cyclohexyl-2-cyclopentyl-34S)-fluoro(4-(trifluoromethyl)phenyl)methyl)-7,7-dimethyl-5,6,7,8-tetrahydroquinolin-5-ol; (S)-4-cyclohexyl-2-cyclopentyl-7,7-dimethyl-3-(4-(trifluoromethyl)benzyl)-5,6,7,8-tetrahydroquinolin-5-ol; (S)-6′(S)-fluoro(4-(trifluoromethyl)phenyl)methyl)-5′-(4-fluorophenyl)-7′-isopropyl-3′,4′-dihydrospiro[cyclobutane-1,2′-pyrano[2,3-b]pyridin]-4′-ol; (S)-6′-((S)-fluoro(4-(trifluoromethyl)phenyl)methyl)-5′-(4-fluorophenyl)-7′-isopropyl-3′,4′-dihydrospiro[cyclopropane-1,2′-pyrano[2,3-b]pyridin]-4′-ol; (S)-5′-(4-fluorophenyl)-6′(S)-hydroxy(4-(trifluoromethyl)phenyl)methyl)-7′-isopropyl-3′,4′-dihydrospiro[cyclobutane-1,2′-pyrano[2,3-b]pyridin]-4′-ol; (S)-5′-(4-fluorophenyl)-6′-((S)-hydroxy(4-(trifluoromethyl)phenyl)methyl)-7′-isopropyl-3′,4′-dihydrospiro[cyclopropane-1,2′-pyrano[2,3-b]pyridin]-4′-ol; (S)-(2-cyclopentyl-5-hydroxy-4-isopropyl-7,7-dimethyl-5,6,7,8-tetrahydroquinolin-3-yl)(4-(trifluoromethyl)phenyl)methanone; (S)-(2-cyclopentyl-5-hydroxy-7,7-dimethyl-4-(penta-1,3-diyn-1-yl)-5,6,7,8-tetrahydroquinolin-3-yl)(4-(trifluoromethyl)phenyl)methanone compound with dihydrogen (1:3); (S)-(2-cyclopentyl-4-(hexa-1,3,5-triyn-1-yl)-5-hydroxy-7,7-dimethyl-5,6,7,8-tetrahydroquinolin-3-yl)(4-(trifluoromethyl)phenyl)methanone compound with dihydrogen (1:5); (S)-(2′-cyclopentyl-5′-hydroxy-4′-isopropyl-5′,8′-dihydro-6′H-spiro[cyclobutane-1,7′-quinolin]-3′-yl)(4-(trifluoromethyl)phenyl)methanone; (S)-(2′-cyclopentyl-5′-hydroxy-4′-(penta-1,3-diyn-1-yl)-5′,8′-dihydro-6′H-spiro[cyclobutane-1,7′-quinolin]-3′-yl)(4-(trifluoromethyl)phenyl)methanone compound with dihydrogen (1:3); (S)-(2′-cyclopentyl-4′-(hexa-1,3,5-triyn-1-yl)-5′-hydroxy-5′,8′-dihydro-6′H-spiro[cyclobutane-1,7′-quinolin]-3′-yl)(4-(trifluoromethyl)phenyl)methanone compound with dihydrogen (1:5); (S)-(4-cyclohexyl-5-hydroxy-2-isopropyl-7,7-dimethyl-5,6,7,8-tetrahydroquinolin-3-yl)(4-(trifluoromethyl)phenyl)methanone; (S)-(4′-cyclohexyl-5′-hydroxy-2′-isopropyl-5′,8′-dihydro-6′H-spiro[cyclobutane-1,7′-quinolin]-3′-yl)(4-(trifluoromethyl)phenyl)methanone; (S)-4-(4,4-difluorocyclohexyl)-34(S)-fluoro(4-(trifluoromethyl)phenyl)methyl)-2-(1-(5-(3-hydroxy-3-methylbutoxy)pyrimidin-2-yl)piperidin-4-yl)-7,7-dimethyl-5,6,7,8-tetrahydroquinolin-5-ol; N-((2-(44(S)-4-(4,4-difluorocyclohexyl)-3-((S)-fluoro(4-(trifluoromethyl)phenyl)methyl)-5-hydroxy-7,7-dimethyl-5,6,7,8-tetrahydroquinolin-2-yl)piperidin-1-yl)pyrimidin-5-yl)methyl)-N-methylmethanesulfonamide; (S)-4-(4,4-difluorocyclohexyl)-3-((S)-fluoro(4-(trifluoromethyl)phenyl)methyl)-7,7-dimethyl-2-(1-(5-((1-methylpiperidin-4-yl)oxy)pyrimidin-2-yl)piperidin-4-yl)-5,6,7,8-tetrahydroquinolin-5-ol; (S)-6′-(R)-fluoro(4-(trifluoromethyl)phenyl)methyl)-5′-(4-fluorophenyl)-7′-isopropyl-3′,4′-dihydrospiro[cyclobutane-1,2′-pyrano[2,3-b]pyridin]-4′-ol; (S)-6′4(R)-fluoro(4-(trifluoromethyl)phenyl)methyl)-5′-(4-fluorophenyl)-7′-isopropyl-3′,4′-dihydrospiro[cyclopropane-1,2′-pyrano[2,3-b]pyridin]-4′-ol; 2-phenyl-1-(pyridin-2-yl)-1-(3-(trifluoromethyl)phenyl)ethyl 3,3-dimethylbutanoate; (S)-1-(1-(5-chloropyridin-2-yl)-1-(3-fluoro-5-(1,1,2,2-tetrafluoroethoxy)phenyl)-2-phenylethyl)-3-cyclopentylurea; (S)—N-(1-(5-chloropyridin-2-yl)-1-(3-fluoro-5-(1,1,2,2-tetrafluoroethoxy)phenyl)-2-phenylethyl)-4-fluoro-3-(trifluoromethyl)benzamide; 1-((S)-1-(5-chloropyridin-2-yl)-1-(3-fluoro-5-(1,1,2,2-tetrafluoroethoxy)phenyl)-2-phenylethyl)-3-((R)-3,3-difluorocyclopentyl)urea; (S)-1-(1-(5-chloropyridin-2-yl)-1-(3-fluoro-5-(1,1,2,2-tetrafluoroethoxy)phenyl)-2-phenylethyl)-3-(3,3-difluorocyclobutyl)urea; (3′R,9′S)-4′-isopropyl-7′,7′-dimethyl-3′-(4-(trifluoromethyl)phenyl)-6′,7′,8′,9′-tetrahydro-3′H-spiro[cyclopentane-1,1′-furo[3,4-c]quinolin]-9′-ol; (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol; (3′R,6′R,9′S)-4′-isopropyl-3′-(4-(trifluoromethyl)phenyl)-2″,3′,3″,5″,6′,6″,8′,9′-octahydrodispiro[cyclopropane-1,7′-furo[3,4-c]quinoline-1′,4″-pyran]-6′,9′-diol; (S)-1-(1-(5-chloropyridin-2-yl)-1-(3-fluoro-5-(1,1,2,2-tetrafluoroethoxy)phenyl)-2-phenylethyl)-3-(2,2,2-trifluoroethyl)urea; (R)-3-(((S)-3-(5-chloropyridin-2-yl)-3-(3-fluoro-5-(1,1,2,2-tetrafluoroethoxy)phenyl)-4-phenylbutyl)amino)-1,1,1-trifluoropropan-2-ol; (R)-3-(((R)-2-(5-chloropyridin-2-yl)-2-(3-fluoro-5-(1,1,2,2-tetrafluoroethoxy)phenyl)-3-phenylpropyl)amino)-1,1,1-trifluoropropan-2-ol; 5-chloro-6-fluoro-N-(3-(trifluoromethyl)phenethyl)-N-(4-(trimethylsilyl)benzyl)-1H-indole-7-carboxamide; 5-chloro-6-fluoro-N-(3-(trifluoromethoxy)phenethyl)-N-(4-(trimethylsilyl)benzyl)-1H-indole-7-carboxamide; Dacetrapib; N-(4-(tert-butyl)benzyl)-5-chloro-N-(3-(trifluoromethyl)phenethyl)-1H-pyrrolo[2,3-c]pyridine-7-carboxamide; 3,5-dichloro-N-(4-chlorophenethyl)-N-(4-(perfluoropropan-2-yl)benzyl)benzamide; and N-((5-(tert-butyl)thiophen-2-yl)methyl)-5-chloro-2-(methylamino)-N-(4-(trifluoromethyl)phenethyl)nicotinamide; and pharmaceutically acceptable salts of the foregoing.

In some embodiments, the CETP inhibitor is an antibody or peptide. U.S. Pat. No. 5,519,001, herein incorporated by reference, describes a 36 amino acid peptide derived from baboon apo C-1 that inhibits CETP activity. Cho et al. (Biochim. Biophys. Acta (1998) 1391: 133-144) describes a peptide from hog plasma that inhibits human CETP. Bonin et al. (J. Peptide Res. (1998) 51, 216-225) discloses a decapeptide inhibitor of CETP. A depspeptide fungal metabolite is disclosed as a CETP inhibitor by Hedge et al. in Bioorg. Med. Chem. Lett., (1998) 8:1277-80. An anti-CETP antibody has been described in W02013075040 A1, herein incorporated by reference.

### ADCY Inhibitors

An ADCY inhibitor can be a small molecule, anti-ADCY antibody, or peptide that inhibits or suppresses adenylate cyclase expression or activity. In some embodiments, the ADCY inhibitor inhibits or suppresses adenylate cyclase expression or activity of one or more of ADCY1, ADCY2, ADCY3, ADCY4, ADCY5, ADCY6, ADCY7, ADCY8, ADCY9 and ADCY10. in some embodiments, the ADCY inhibitor is an ADCY1, ADCY2, ADCY3, ADCY4, ADCY5, ADCY6, ADCY7, ADCY8, ADCY9, or ADCY10 inhibitor.

The following table lists illustrative ADCY inhibitors. These ADCY inhibitors and pharmaceutically acceptable salts thereof are useful in the methods and compositions of the present invention. Each compound's structure is depicted at the immediate right of its name.

| Compound | Structure |
|---|---|
| SQ 22,536 | 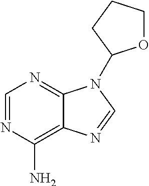 [SMILES: Nc1ncnc2c1ncn2C1CCCO1] |
| NKY80 | 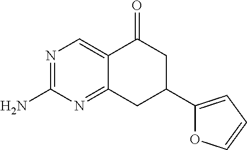 [SMILES: Nc1ncc2c(n1)CC(c1ccco1)CC2=O] |
| vidarabine | 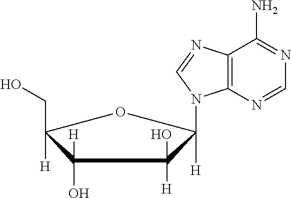 |
| NB001 | 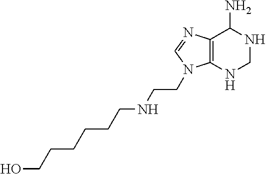 [SMILES: NC1NCNc2c1ncn2CCNCCCCCCO] |
| BODIPY-FS | 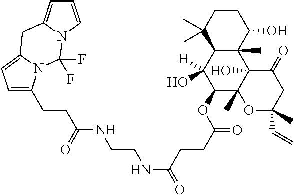 |
| 6A7DA-FS | 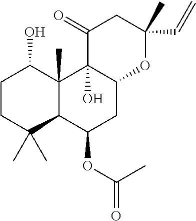 |
| Tyrphostin A25 | 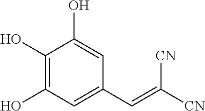 [SMILES: N#CC(C#N)=Cc1cc(O)c(O)c(O)c1] |
| (E)-2-(1H- Benzo[d]imidazol- 2-ylthio)- N′-(5-bromo-2- hydroxy- benzylidene) propanehydrazide | 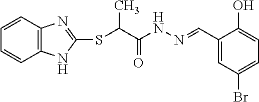 [SMILES: CC(Sc1nc2ccccc2[nH]1)C(=O)N/N=C/c1cc(Br)ccc1O] |
| LRE1 | 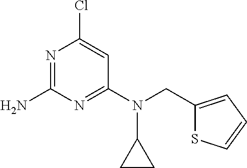 [SMILES: Nc1nc(Cl)cc(N(Cc2cccs2)C2CC2)n1] |
| 2′,5′- Dideoxyadenosine 3′-triphosphate tetrasodium salt | 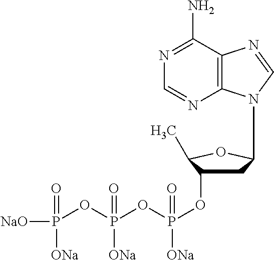 |
| 2′,5′-dd-3′- ATP | 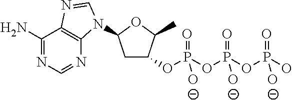 [SMILES: C[C@@H]1O[C@H](n2cnc3c(N)ncnc32)C[C@H]1OP(=O)([O-])OP(=O)([O-])OP(=O)([O-])O] |
| AraAde | 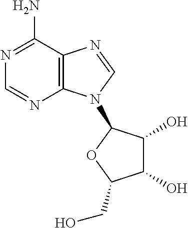 [SMILES: Nc1ncnc2c1ncn2[C@@H]1O[C@@H](CO)[C@@H](O)[C@H]1O] |
| PMC6 | 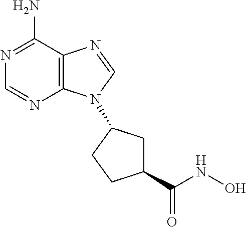 [SMILES: Nc1ncnc2c1ncn2[C@H]1CC[C@H](C(=O)NO)C1] |
| MDL 12330A | 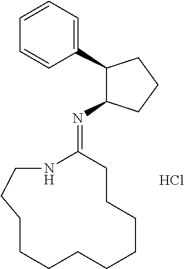 [SMILES: c1ccc([C@H]2CCC[C@H]2/N=C2\CCCCCCCCCCCN2)cc1 ; Cl] |
| 1,9-dd-FS | 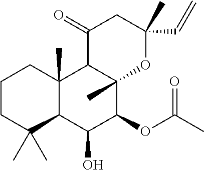 |
| calmidazolium | 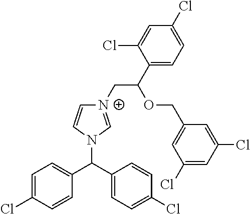 [SMILES: Clc1ccc(C(c2ccc(Cl)cc2)n2cc[n+](CC(OCc3cc(Cl)cc(Cl)c3)c3ccc(Cl)cc3Cl)c2)cc1] |
| 9- Cyclopentyl adenine monomethane- sulfonate | 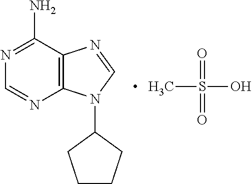 |
| SB-268262 | 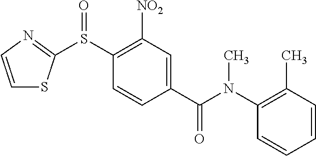 [SMILES: Cc1ccccc1N(C)C(=O)c1ccc(S(=O)c2nccs2)c([N+](=O)[O-])c1] |
| 2′,5′- Dideoxyadenosine | 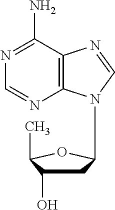 |

Additional ADCY inhibitors useful in the compositions and methods of the present invention are disclosed in Dessauer et al. Pharmacol Rev, (2017) 69 (2): 93-139, and have the structure:

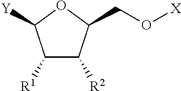

| Compound | R1 | R2 | X | Y |
|---|---|---|---|---|
| MANT- ATP | 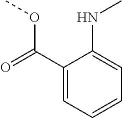 | OH | 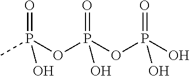 | 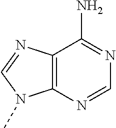 |
| MANT-ITP | 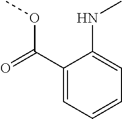 | OH | 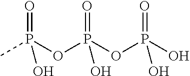 | 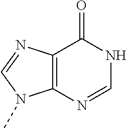 |
| MANT- GTP | 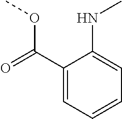 | OH | 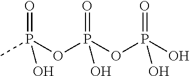 | 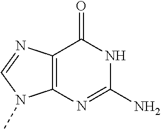 |
| MANT- XTP | 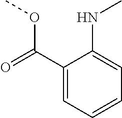 | OH | 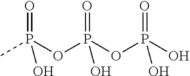 | 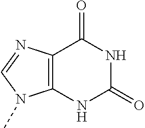 |
| MANT- CTP | 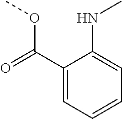 | OH | 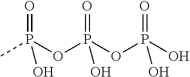 | 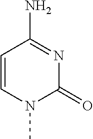 |
| MANT- UTP | 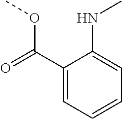 | OH | 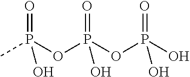 | 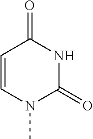 |
| 2′-MANT- 3′dATP | H | 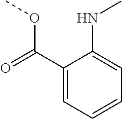 | 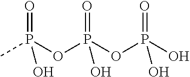 | 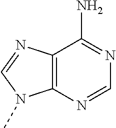 |
| 3′-MANT- 2′dATP | 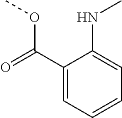 | H | 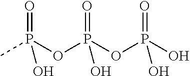 | 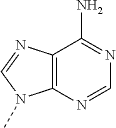 |
| MANT- ATPγS | 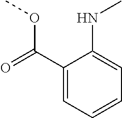 | OH | 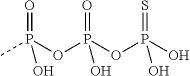 | 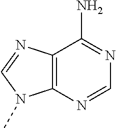 |
| MANT- ITPγS | 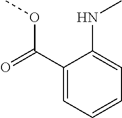 | OH | 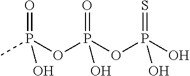 | 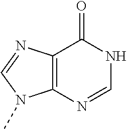 |
| MANT- GTPγS | 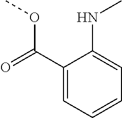 | OH | 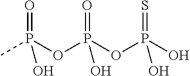 | 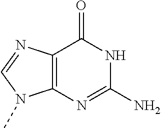 |
| MANT- UTPγS | 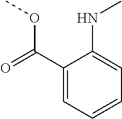 | OH | 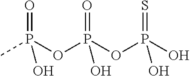 | 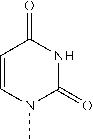 |
| ANT-ATP | 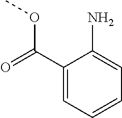 | OH | 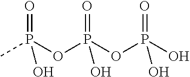 | 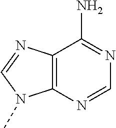 |
| Cl-ANT- ATP | 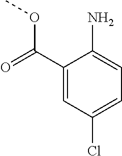 | OH | 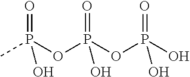 | 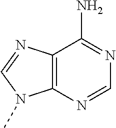 |
| Cl-ANT- ITP | 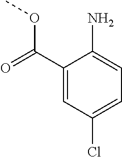 | OH | 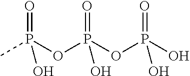 | 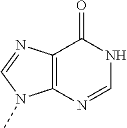 |
| Br-ANT- ITP | 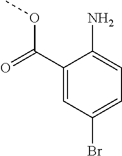 | OH | 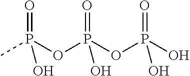 | 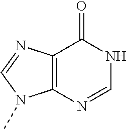 |
| Pr-ANT- ATP | 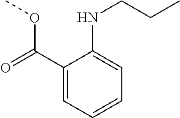 | OH | 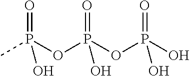 | 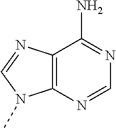 |
| Pr-ANT- ITP | 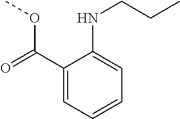 | OH | 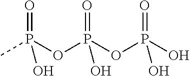 | 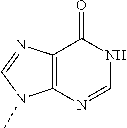 |
| AcNH- ANT-ATP | 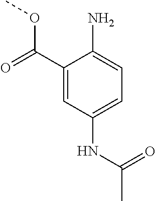 | OH | 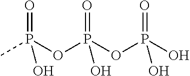 | 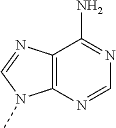 |
| AcNH- ANT-ITP | 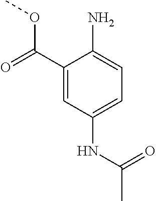 | OH | 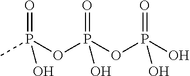 | 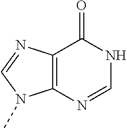 |
| MANT- AppNHp | 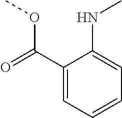 | OH | 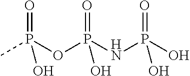 | 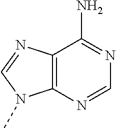 |
| MANT- GppNHp | 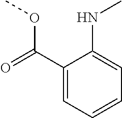 | OH | 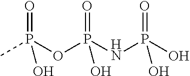 | |
| TNP-ATP | 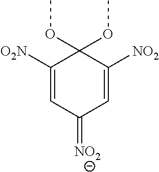 | | 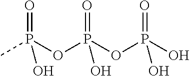 | 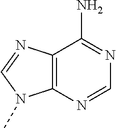 |
| TNP-GTP | 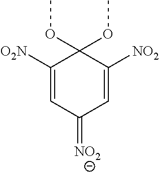 | | 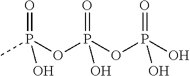 | 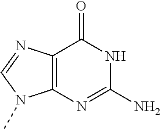 |
| TNP-CTP | 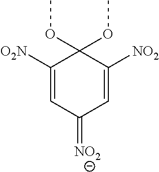 | | 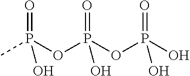 | 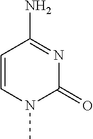 |
| TNP-UTP | 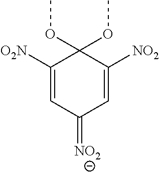 | | 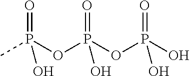 | 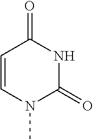 |
| Bis- MANT- ATP | 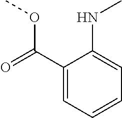 | 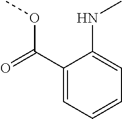 | 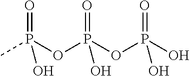 | 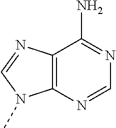 |
| Bis- MANT-ITP | 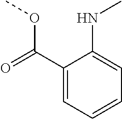 | 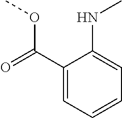 | 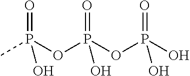 | 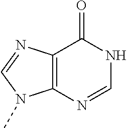 |
| Bis- MANT- CTP | 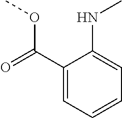 | 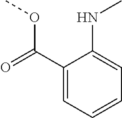 | 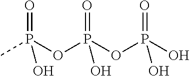 | 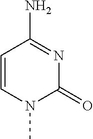 |
| Bis- MANT- IDP | 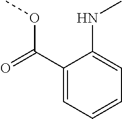 | 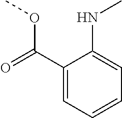 | 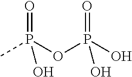 | 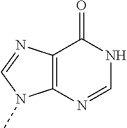 |
| Bis- MANT- IMP | 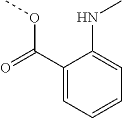 | 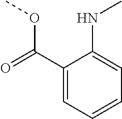 | 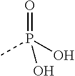 | 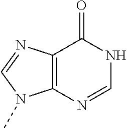 |
| Bis-Cl- ANT-ATP | 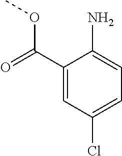 | 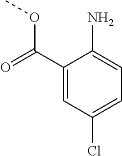 | 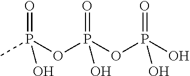 | 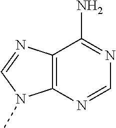 |
| Bis-Cl- ANT-ITP | 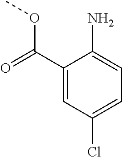 | 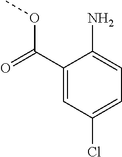 | 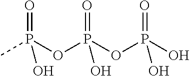 | 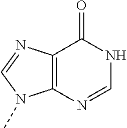 |
| Bis-Br- ANT-ATP | 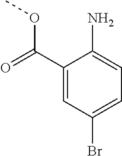 | 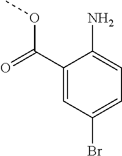 | 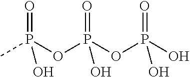 | 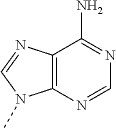 |
| Bis-Br- ANT-ITP | 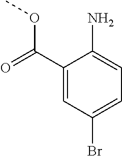 | 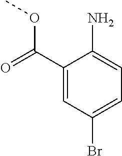 | 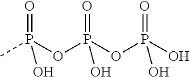 | 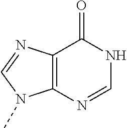 |
| Bis-Pr- ANT-ATP | 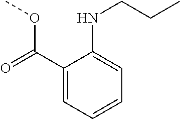 | 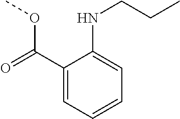 | 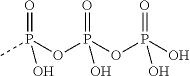 | 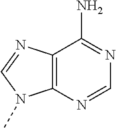 |
| Bis-Pr- ANT-ITP | 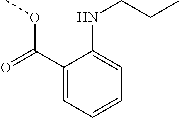 | 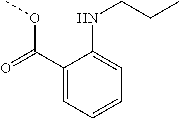 | 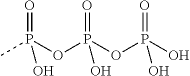 | 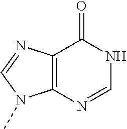 |
| Bis-AcNH- ANT-ATP | 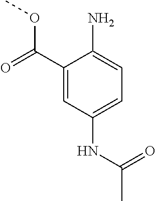 | 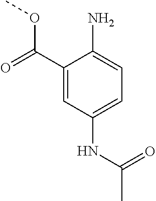 | 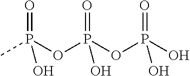 | 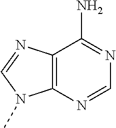 |
| Bis-AcNH- ANT-ITP | 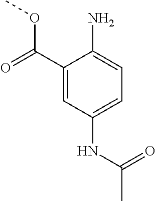 | 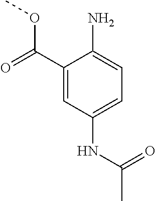 | 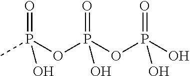 | 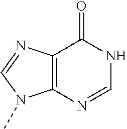 |

and pharmaceutically acceptable salts of the foregoing.

Additional examples of small molecule ADCY inhibitors include, but are not limited to: SQ22536 (9-(tetrahydro-2-furanyl)-adenine); 2′,5′-dideoxyadenosine, 9-cyclopentyladenine; 2′,5′-dideoxyadenosine 3′-diphosphate; 2′,5′-dideoxyadenosine 3′-monophosphate; MDL-12330A (cis-N-(2-phenylcyclopentyl)azacyclotridece-1-en-2-amine); 2-amino-7-(4-chlorophenyl)-7,8-dihydro-5(6H)-quinazolinone; 2-amino-7-(4-methoxyphenyl)-7,8-dihydro-5(6H)-quinazolinone; 2-amino-7-phenyl-7,8-dihydro-5(6H)-quinazolinone; 4.2-amino-7-(2-furanyl)-7,8-dihydro-5(6H)-quinazolinone; 2-amino-7-(2-thienyl)-7,8-dihydro-5(6H)-quinazolinone); MANT-ATP; MANT-ITP; MANT-GTP; MANT-XTP; MANT-CTP; MANT-UTP; 2′-MANT-3′dATP; 3′-MANT-2′dATP; MANT-ATPγS; MANT-ITPγS; MANT-GTPγS; MANT-UTPyS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-IMP; Bis-Cl-ANT-ATP; Bis-Cl-ANT-ITP; Bis-Br-ANT-ATP; Bis-Br-ANT-ITP; Bis-Pr-ANT-ATP; Bis-Pr-ANT-ITP; Bis-AcNH-ANT-ATP; Bis-AcNH-ANT-ITP; NKY80; vidarabine; 2′,5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; 2′,5′-Dideoxyadenosine 3′-triphosphate tetrasodium salt; and pharmaceutically acceptable salts of the foregoing.

Illustrative ADCY inhibitor peptides useful in the compositions and methods of the present invention include, but are not limited to: adrenocorticotropic hormone; brain natriuretic peptide (BNP); and pituitary adenylate cyclase-activating polypeptide.

### Pharmaceutically Acceptable Salts

Pharmaceutically acceptable salts include, for example, acid-addition salts and base-addition salts. The acid that forms an acid-addition salt can be an organic acid or an inorganic acid. A base that forms a base-addition salt can be an organic base or an inorganic base. In some embodiments, a pharmaceutically acceptable salt is a metal salt. In some embodiments, a pharmaceutically acceptable salt is an ammonium salt.

Acid-addition salts can arise from the addition of an acid to the free-base form of a compound useful in the compositions and methods of the present invention. In some embodiments, the acid is organic. In some embodiments, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid-addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to a compound having a carboxyl group. The inorganic base can include a metal cation paired with a basic counterion, such as, for example, hydroxide, carbonate, bicarbonate, or phosphate. The metal can be an alkali metal, alkaline earth metal, transition metal, or main group metal. Non-limiting examples of suitable metals include lithium, sodium, potassium, cesium, cerium, magnesium, manganese, iron, calcium, strontium, cobalt, titanium, aluminum, copper, cadmium, and zinc.

Non-limiting examples of suitable metal salts include a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, and a zinc salt.

Ammonium salts can arise from the addition of ammonia or an organic amine to a compound having a carboxyl group. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include a triethylammonium salt, a diisopropylammonium salt, an ethanolammonium salt, a diethanolammonium salt, a triethanolammonium salt, a morpholinium salt, an N-methylmorpholinium salt, a piperidinium salt, an N-methylpiperidinium salt, an N-ethylpiperidinium salt, a dibenzylammonium salt, a piperazinium salt, a pyridinium salt, a pyrrazolium salt, an imidazolium salt, a pyrazinium salt, an ethylenediammonium salt, an N,N′-dibenzylethylenediammonium salt, a procaine salt, a chloroprocaine salt, a choline salt, a dicyclohexylammonium salt, and a N-methylglucamine salt.

### Methods for Treating or Preventing a Cardiovascular Disorder

The present invention provides methods for treating or preventing a cardiovascular disorder, comprising administering to a subject in need thereof an effective amount of: a) a CETP inhibitor; and b) an ADCY inhibitor.

In certain embodiments, the cardiovascular disorder is acute coronary syndrome (ACS), atherosclerosis, peripheral vascular disease, dyslipidemia, hyperbetalipoproteinemia, hypoalphalipoproteinemia, hypercholesterolemia, hypertriglyceridemia, familial-hypercholesterolemia, angina, ischemia, cardiac ischemia, stroke, myocardial infarction, reperfusion injury, angioplastic restenosis, hypertension, cardiovascular disease, coronary heart disease, coronary artery disease, hyperlipidemia, hyperlipidoproteinemia or a vascular complication of diabetes, obesity or endotoxemia.

In certain embodiments, administering the CETP inhibitor occurs before, concurrently with, or after administering the ADCY inhibitor. In some embodiments, each of the CETP inhibitor and ADCY inhibitor is present in a separate composition. In some embodiments, the CETP inhibitor and ADCY inhibitor are present in the same composition.

In certain embodiments, the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

In certain embodiments, the ADCY inhibitor is an ADCY1, ADCY2, ADCY3, ADCY4, ADCY5, ADCY6, ADCY7, ADCY8, ADCY9 or ADCY10 inhibitor. In certain embodiments, the ADCY inhibitor is 9-(tetrahydro-2-furanyl)-adenine); 2′,5′-dideoxyadenosine; 9-cyclopentyladenine; 2′,5′-dideoxyadenosine 3′-diphosphate; 2′,5′-dideoxyadenosine 3′-monophosphate; cis-N-(2-phenylcyclopentyl)azacyclotridece-1-en-2-amine); 2-amino-7-(4-chlorophenyl)-7,8-dihydro-5 (6H)-quinazolinone; 2-amino-7-(4-methoxyphenyl)-7,8-dihydro-5(6H)-quinazolinone; 2-amino-7-phenyl-7,8-dihydro-5(6H)-quinazolinone; 2-amino-7-(2-furanyl)-7,8-dihydro-5(6H)-quinazolinone; 2-amino-7-(2-thienyl)-7,8-dihydro-5(6H)-quinazolinone); 2-amino-7-(4-methoxyphenyl)-7,8-dihydro-5(6H)-quinazolinone, 2-amino-7-phenyl-7,8-dihydro-5(6H)-quinazolinone, 4.2-amino-7-(2-furanyl)-7,8-dihydro-5(6H)-quinazolinone, and 2-amino-7-(2-thienyl)-7,8-dihydro-5(6H)-quinazolinone), MANT-ATP; MANT-ITP; MANT-GTP; MANT-XTP; MANT-CTP; MANT-UTP; 2′-MANT-3′dATP; 3′-MANT-2′dATP; MANT-ATPγS; MANT-ITPγS; MANT-GTPγS; MANT-UTPγS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-IMP; Bis-Cl-ANT-ATP; Bis-Cl-ANT-ITP; Bis-Br-ANT-ATP; Bis-Br-ANT-ITP; Bis-Pr-ANT-ATP; Bis-Pr-ANT-ITP; Bis-AcNH-ANT-ATP; Bis-AcNH-ANT-ITP; NKY80; vidarabine; 2′,5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-althio)-N-′-(5-bromo-2-hydroxybenzylidene)propanehydrazide; SB-268262; LRE1; 2′,5′-Dideoxyadenosine; 2′,5′-Dideoxyadenosine 3′-triphosphate tetrasodium salt; adrenocorticotropic hormone; brain natriuretic peptide (BNP); or pituitary adenylate cyclase-activating polypeptide; or a pharmaceutically acceptable salt of any of the foregoing.

In certain embodiments, the subject is known to have genotype rs11647778/CC, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs8049452/GG, rs8061182/AA, rs2238448/TT, rs12920508/GG, rs2531971/AA, or rs12599911/GG. In some embodiments, the subject is known to have genotype rs1967309/AA.

In certain embodiments, the subject is known to have genotype rs11647778/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs8049452/GA, rs8061182/AG, rs2238448/TC, rs12920508/CG, rs2531971/AC, or rs12599911/GT. In some embodiments, the subject is known to have genotype rs1967309/AG.

In certain embodiments, the subject is known to have genotype rs11647778/GG, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs8049452/AA, rs8061182/GG, rs2238448/CC, rs12920508/CC, rs2531971/CC, or rs12599911/TT.

Another aspect of the invention provides methods for treating or preventing a cardiovascular disorder, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor.

In some embodiments, the ADCY is ADCY1, ADCY2, ADCY3, ADCY4, ADCY5, ADCY6, ADCY7, ADCY8, or ADCY10, and the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

In some embodiments, the ADCY is ADCY9, and wherein the CETP inhibitor is BMS795311, CP-800,569, JNJ-28545595, JNJ-28614872, BAY 19-4789, or BAY 38-1315, or a pharmaceutically acceptable salt of any of the foregoing.

In certain embodiments, the control level is an expression or activity level of ADCY established based on the expression or activity level of ADCY of subjects who do not respond positively to treatment of a CETP inhibitor in the absence of an ADCY inhibitor. In certain embodiments, reduced expression or activity level of ADCY in the subject compared to a control level is caused by one or more polymorphisms or mutations in the ADCY gene of the subject. In some embodiments, the ADCY is ADCY1, ADCY2, ADCY3, ADCY4, ADCY5, ADCY6, ADCY7, ADCY8, ADCY9 or ADCY10. In some embodiments, the ADCY is ADCY9.

The activity level of ADCY can be detected using techniques known in the art. For example, adenylate cyclase assays have been disclosed by Salomon et al., (1974) Analytical Biochemistry, 58(2): 541-548; Wiegn et al., (1993) Anal Biochem. 208(2):217-22; and Storm et al., (1998) Neuron, 20:1199-1210.

In some embodiments, expression level of ADCY is determined at protein expression level. In some embodiments, protein expression level of ADCY is analyzed using a specific antibody and a protein assay. Any suitable method or assay can be used to measure the level of ADCY protein expression in the biological sample of a subject. Numerous antibody-based detection formats are well known in the art, and include ELISA (enzyme linked immunosorbent assay), radioimmunoassays, immunoblots, Western blots, flow cytometry, immunofluorescence assays, immunoprecipitation, protein A assays, immunoelectrophoresis assays, and other related techniques. In some embodiments, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the kits, assays and methods described herein. Antibodies specific for ADCY may be provided in a diagnostic kit that incorporates at least one of these procedures to quantitate ADCY expression. The kit may contain other components, packaging, instructions, or other material to aid the quantitation of the protein and use of the kit.

Anti-ADCY antibodies as described herein can be obtained commercially or routinely made according to methods such as, but not limited to, inoculation of an appropriate animal with the polypeptide or an antigenic fragment, in vitro stimulation of lymphocyte populations, synthetic methods, hybridomas, and/or recombinant cells expressing nucleic acid encoding such anti-ADCY antibodies. Immunization of an animal using purified recombinant ADCY or peptide fragments thereof, is an example of a method of preparing anti-ADCY antibodies. Similarly, immunization of an animal using purified recombinant ADCY or peptide fragments thereof, is an example of a method of preparing anti-ADCY antibodies.

In other embodiments, level of ADCY is analyzed at the mRNA level. For example, RT-PCR and a pair of specific primers may be used. mRNA are prepared and analyzed according to well-established protocols.

In certain embodiments, reduced expression or activity level of ADCY in the subject is tissue or cell type specific. In some embodiments, the subject is known to have reduced expression or activity level of ADCY in the subject's central nervous system compared to a control level. In some embodiments, the subject is known to have reduced expression or activity level of ADCY in the subject's hypothalamus compared to a control level. In some embodiments, the ADCY is ADCY1, ADCY2, ADCY3, ADCY4, ADCY5, ADCY6, ADCY7, ADCY8, ADCY9 or ADCY10. In some embodiments, the ADCY is ADCY9.

In some embodiments, the subject is known to have reduced expression or activity level of ADCY in the subject's central nervous system compared to a control level. In some embodiments, the subject is known to have reduced expression or activity level of ADCY in the subject's hypothalamus compared to a control level.

In some embodiments, the subject of the forgoing methods is a human. In some embodiments, the subject is an adult human. In some embodiments, the subject is a pediatric human.

In certain embodiments, the cardiovascular disorder is acute coronary syndrome (ACS), atherosclerosis, peripheral vascular disease, dyslipidemia, hyperbetalipoproteinemia, hypoalphalipoproteinemia, hypercholesterolemia, hypertriglyceridemia, familial-hypercholesterolemia, angina, ischemia, cardiac ischemia, stroke, myocardial infarction, reperfusion injury, angioplastic restenosis, hypertension, cardiovascular disease, coronary heart disease, coronary artery disease, hyperlipidemia, hyperlipidoproteinemia or a vascular complication of diabetes, obesity or endotoxemia.

In certain embodiments, the subject has acute coronary syndrome (ACS).

In certain embodiments, the CETP inhibitor is dalcetrapib, torcetrapib, anacetrapib, evacetrapib, obicetrapib, BMS795311, CP-800,569, DLB S-1449, ATH-03, DRL-17822, JNJ-28545595, JNJ-28614872, BAY 19-4789, BAY 38-1315, or BAY 60-5521, or a pharmaceutically acceptable salt of any of the foregoing. In other embodiments, the CETP inhibitor is BMS795311, CP-800,569, JNJ-28545595, JNJ-28614872, BAY 19-4789, BAY 38-1315, or a pharmaceutically acceptable salt of any of the foregoing.

In certain embodiments, the ADCY inhibitor is an ADCY1, ADCY2, ADCY3, ADCY4, ADCY5, ADCY6, ADCY7, ADCY8, ADCY9 or ADCY10 inhibitor. In certain embodiments, the ADCY inhibitor is SQ22536 (9-(tetrahydro-2-furanyl)-adenine), 2′,5′-dideoxyadenosine, 9-cyclopentyladenine, 2′,5′-dideoxyadenosine 3′-diphosphate, 2′,5′-dideoxyadenosine 3′-monophosphate, MDL-12330A (cis-N-(2-phenylcyclopentyl)azacyclotridece-1-en-2-amine), compounds such as 7,8-dihydro-5(6H)-quinazolinone derivatives disclosed in JP Patent Application No. 2001-153954 (preferably, 2-amino-7-(4-chlorophenyl)-7, 8-dihydro-5 (6H)-quinazolinone, 2-amino-7-(4-methoxyphenyl)-7,8-dihydro-5(6H)-quinazolinone, 2-amino-7-phenyl-7,8-dihydro-5(6H)-quinazolinone, 4.2-amino-7-(2-furanyl)-7,8-dihydro-5(6H)-quinazolinone, and 2-amino-7-(2-thienyl)-7,8-dihydro-5(6H)-quinazolinone), MANT-ATP; MANT-ITP; MANT-GTP; MANT-XTP; MANT-CTP; MANT-UTP; 2′-MANT-3′dATP; 3′-MANT-2′dATP; MANT-ATPγS; MANT-ITPγS; MANT-GTPyS; MANT-UTPyS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-IMP; Bis-Cl-ANT-ATP; Bis-Cl-ANT-ITP; Bis-Br-ANT-ATP; Bis-Br-ANT-IPT; Bis-Pr-ANT-ATP; Bis-Pr-ANT-ITP; Bis-AcNH-ANT-ATP; Bis-AcNH-ANT-ITP; NKY80; vidarabine; 2′,5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; 2′,5′-Dideoxyadenosine 3′-triphosphate tetrasodium salt; or a pharmaceutically acceptable salt of any of the foregoing.

In some embodiments, the ADCY inhibitor is adrenocorticotropic hormone; brain natriuretic peptide (BNP); or pituitary adenylate cyclase-activating polypeptide.

In certain embodiments, the subject is known to have genotype rs11647778/CC, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs8049452/GG, rs8061182/AA, rs2238448/TT, rs12920508/GG, rs2531971/AA, or rs12599911/GG. In some embodiments, the subject is known to have genotype rs1967309/AA.

In certain embodiments, the subject is known to have genotype rs11647778/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs8049452/GA, rs8061182/AG, rs2238448/TC, rs12920508/CG, rs2531971/AC, or rs12599911/GT. In some embodiments, the subject is known to have genotype rs1967309/AG.

In certain embodiments, the subject is known to have genotype rs11647778/GG, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs8049452/AA, rs8061182/GG, rs2238448/CC, rs12920508/CC, rs2531971/CC, or rs12599911/TT.

The invention further provides methods for treating or preventing a cardiovascular disorder, comprising administering to a subject in need thereof an effective amount of a CETP inhibitor, wherein the CEPT inhibitor is BMS795311, CP-800,569, JNJ-28545595, JNJ-28614872, BAY 19-4789, BAY 38-1315, or a pharmaceutically acceptable salt of any of the foregoing and wherein the subject is known to have genotype rs11647778/CC, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs8049452/GG, rs8061182/AA, rs2238448/TT, rs12920508/GG, rs2531971/AA, or rs12599911/GG. In some embodiments, the subject is known to have genotype rs1967309/AA.

The invention further provides methods for treating or preventing a cardiovascular disorder, comprising administering to a subject in need thereof an effective amount of a CETP inhibitor, wherein the CEPT inhibitor is BMS795311, CP-800,569, JNJ-28545595, JNJ-28614872, BAY 19-4789, BAY 38-1315, or a pharmaceutically acceptable salt of any of the foregoing and wherein the subject is known to have genotype rs11647778/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs8049452/GA, rs8061182/AG, rs2238448/TC, rs12920508/CG, rs2531971/AC, or rs12599911/GT. In some embodiments, the subject is known to have genotype rs1967309/AG.

The invention further provides methods for treating or preventing a cardiovascular disorder, comprising administering to a subject in need thereof an effective amount of a CETP inhibitor, wherein the CEPT inhibitor is BMS795311, CP-800,569, JNJ-28545595, JNJ-28614872, BAY 19-4789, BAY 38-1315, or a pharmaceutically acceptable salt of any of the foregoing and wherein the subject is known to have genotype rs11647778/GG, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs8049452/AA, rs8061182/GG, rs2238448/CC, rs12920508/CC, rs2531971/CC, or rs12599911/TT.

Methods for determining whether a subject has a particular genotype are well known to a person skilled in the art, see, e.g., WO 2014/154606A1 and WO 2016/016157A1.

### Methods for Reducing Risk of a Cardiovascular Event

The present invention further provides methods for reducing risk of a cardiovascular event, comprising administering to a subject in need thereof an effective amount of: a) a CETP inhibitor; and b) an ADCY inhibitor.

In certain embodiments, administering the CETP inhibitor occurs before, concurrently with, or after administering the ADCY inhibitor. In some embodiments, each of the CETP inhibitor and ADCY inhibitor is present in a separate composition. In some embodiments, the CETP inhibitor and ADCY inhibitor are present in the same composition.

The present invention further provides methods for reducing the risk of a cardiovascular event, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor.

In certain embodiments, the control level is an expression or activity level of ADCY established based on the expression or activity level of ADCY of subjects who do not respond positively to treatment of a CETP inhibitor in the absence of an ADCY inhibitor. In certain embodiments, reduced expression or activity level of ADCY in the subject compared to a control level is caused by one or more polymorphisms or mutations in the ADCY gene of the subject. In some embodiments, the ADCY is ADCY1, ADCY2, ADCY3, ADCY4, ADCY5, ADCY6, ADCY7, ADCY8, ADCY9 or ADCY10. In some embodiments, the ADCY is ADCY9. Methods to measure the expression or activity level of ADCY are disclosed herein.

In some embodiments, the ADCY is ADCY1, ADCY2, ADCY3, ADCY4, ADCY5, ADCY6, ADCY7, ADCY8, or ADCY10, and the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

In some embodiments, the ADCY is ADCY9, and wherein the CETP inhibitor is BMS795311, CP-800,569, JNJ-28545595, JNJ-28614872, BAY 19-4789, or BAY 38-1315, or a pharmaceutically acceptable salt of any of the foregoing.

In certain embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization.

In certain embodiments, the cardiovascular event is an adverse cardiovascular event, e.g., coronary heart disease, death, cardiac arrest, myocardial infarction, ischemic stroke, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In certain embodiments, the subject has acute coronary syndrome (ACS).

In certain embodiments, the CETP inhibitor is dalcetrapib, torcetrapib, anacetrapib, evacetrapib, obicetrapib, BMS795311, CP-800,569, DLB S-1449, ATH-03, DRL-17822, JNJ-28545595, JNJ-28614872, BAY 19-4789, BAY 38-1315, or BAY 60-5521, or a pharmaceutically acceptable salt of any of the foregoing. In other embodiments, the CETP inhibitor is BMS795311, CP-800,569, JNJ-28545595, JNJ-28614872, BAY 19-4789, or BAY 38-1315, or a pharmaceutically acceptable salt of any of the foregoing.

In certain embodiments, the ADCY inhibitor is an ADCY1, ADCY2, ADCY3, ADCY4, ADCY5, ADCY6, ADCY7, ADCY8, ADCY9 or ADCY10 inhibitor. In certain embodiments, the ADCY inhibitor is SQ22536 (9-(tetrahydro-2-furanyl)-adenine), 2′,5′-dideoxyadenosine, 9-cyclopentyladenine, 2′,5′-dideoxyadenosine 3′-diphosphate, 2′,5′-dideoxyadenosine 3′-monophosphate, MDL-12330A (cis-N-(2-phenylcyclopentyl)azacyclotridece-1-en-2-amine), compounds such as 7,8-dihydro-5(6H)-quinazolinone derivatives disclosed in JP Patent Application No. 2001-153954 (preferably, 2-amino-7-(4-chlorophenyl)-7, 8-dihydro-5 (6H)-quinazolinone, 2-amino-7-(4-methoxyphenyl)-7,8-dihydro-5(6H)-quinazolinone, 2-amino-7-phenyl-7,8-dihydro-5(6H)-quinazolinone, 4.2-amino-7-(2-furanyl)-7,8-dihydro-5(6H)-quinazolinone, and 2-amino-7-(2-thienyl)-7,8-dihydro-5(6H)-quinazolinone), MANT-ATP; MANT-ITP; MANT-GTP; MANT-XTP; MANT-CTP; MANT-UTP; 2′-MANT-3′dATP; 3′-MANT-2′dATP; MANT-ATPγS; MANT-ITPγS; MANT-GTPγS; MANT-UTPγS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-IMP; Bis-Cl-ANT-ATP; Bis-Cl-ANT-ITP; Bis-Br-ANT-ATP; Bis-Br-ANT-ITP; Bis-Pr-ANT-ATP; Bis-Pr-ANT-ITP; Bis-AcNH-ANT-ATP; Bis-AcNH-ANT-ITP; NKY80; vidarabine; 2′, 5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; 2′,5′-Dideoxyadenosine 3′-triphosphate tetrasodium salt; or a pharmaceutically acceptable salt of any of the foregoing.

In some embodiments, the ADCY inhibitor is adrenocorticotropic hormone; brain natriuretic peptide (BNP); or pituitary adenylate cyclase-activating polypeptide.

In certain embodiments, the subject is known to have genotype rs11647778/CC, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs8049452/GG, rs8061182/AA, rs2238448/TT, rs12920508/GG, rs2531971/AA, or rs12599911/GG. In some embodiments, the subject is known to have genotype rs1967309/AA.

In certain embodiments, the subject is known to have genotype rs11647778/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs8049452/GA, rs8061182/AG, rs2238448/TC, rs12920508/CG, rs2531971/AC, or rs12599911/GT. In some embodiments, the subject is known to have genotype rs1967309/AG.

In certain embodiments, the subject is known to have genotype rs11647778/GG, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs8049452/AA, rs8061182/GG, rs2238448/CC, rs12920508/CC, rs2531971/CC, or rs12599911/TT.

The invention further provides methods for lowering risk of a cardiovascular event, comprising administering to a subject in need thereof an effective amount of a CETP inhibitor, wherein the CEPT inhibitor is BMS795311, CP-800,569, JNJ-28545595, JNJ-28614872, BAY 19-4789, or BAY 38-1315, or a pharmaceutically acceptable salt of any of the foregoing and wherein the subject is known to have genotype rs11647778/CC, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs8049452/GG, rs8061182/AA, rs2238448/TT, rs12920508/GG, rs2531971/AA, or rs12599911/GG. In some embodiments, the subject is known to have genotype rs1967309/AA.

The invention further provides methods for lowering risk of a cardiovascular event, comprising administering to a subject in need thereof an effective amount of a CETP inhibitor, wherein the CEPT inhibitor is BMS795311, CP-800,569, JNJ-28545595, JNJ-28614872, BAY 19-4789, or BAY 38-1315, or a pharmaceutically acceptable salt of any of the foregoing and wherein the subject is known to have genotype rs11647778/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs8049452/GA, rs8061182/AG, rs2238448/TC, rs12920508/CG, rs2531971/AC, or rs12599911/GT. In some embodiments, the subject is known to have genotype rs1967309/AG.

The invention further provides methods for lowering risk of a cardiovascular event, comprising administering to a subject in need thereof an effective amount of a CETP inhibitor, wherein the CEPT inhibitor is BMS795311, CP-800,569, JNJ-28545595, JNJ-28614872, BAY 19-4789, or BAY 38-1315, or a pharmaceutically acceptable salt of any of the foregoing and wherein the subject is known to have genotype rs11647778/GG, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs8049452/AA, rs8061182/GG, rs2238448/CC, rs12920508/CC, rs2531971/CC, or rs12599911/TT.

### Dosages

The dosage of the CETP inhibitors and ADCY inhibitors of the methods and compositions of the invention can be selected in accordance with a variety of factors including type, species, age, weight, sex and medical condition of the subject; the severity of the disorder to be treated or prevented; the severity of the cardiovascular event; the route of administration; the renal or hepatic function of the subject; or the CETP or ADCY inhibitor to be administered.

In some embodiments, the daily dosage amount of CETP inhibitor useful in the methods and compositions of the present invention ranges from about 1 mg to about 1000 mg.

In some embodiments, the daily dosage amount of ADCY inhibitor useful in the methods and compositions of the present invention ranges from about 1 mg to about 1000 mg.

In certain embodiments, the CETP inhibitor is dalcetrapib or a pharmaceutically acceptable salt thereof, wherein dalcetrapib or pharmaceutically acceptable salt thereof is administered orally at a dose of about 200 mg, 300 mg, 400 mg, 500 mg, 600 mg, 700 mg, 800 mg, 900 mg, or 1000 mg daily.

In certain embodiments, the CETP inhibitor is torcetrapib or a pharmaceutically acceptable salt thereof, wherein torcetrapib or pharmaceutically acceptable salt thereof is administered orally at a dose of about 20 mg, 30 mg, 40 mg, 50 mg, 60 mg, 70 mg, 80 mg, 90 mg, or 100 mg daily.

In certain embodiments, the CETP inhibitor is anacetrapib or a pharmaceutically acceptable salt thereof, wherein anacetrapib or pharmaceutically acceptable salt thereof is administered orally at a dose of about 40 mg, 60 mg, 80 mg, 100 mg, 120 mg, 140 mg, 160 mg, 180 mg, or 200 mg daily.

In certain embodiments, the CETP inhibitor is evacetrapib a pharmaceutically acceptable salt thereof, wherein evacetrapib or pharmaceutically acceptable salt thereof is administered orally at a dose of about 30 mg, 60 mg, 90 mg, 100 mg, 120 mg, 140 mg, 160 mg, 180 mg, 200 mg, 250 mg, 300 mg, 350 mg, 400 mg, 450 mg, 500 mg, 550 mg, or 600 mg daily.

In certain embodiments, the CETP inhibitor is BAY 60-5521 or a pharmaceutically acceptable salt thereof, wherein BAY 60-5521 or pharmaceutically acceptable salt thereof is administered orally at a dose of about 5 mg, 12.5 mg, 25 mg, 30mg, 40mg, 50 mg, 60 mg, 70 mg, 80 mg, 90 mg, or 100 mg daily.

In some embodiments, the subject of the forgoing methods is a human. In some embodiments, the subject is an adult human. In some embodiments, the subject is a pediatric human.

### Compositions and Kits

The present invention also provides compositions comprising a) an effective amount of a CETP inhibitor and an ADCY inhibitor; and b) a pharmaceutically acceptable carrier or vehicle.

In certain embodiments, the CETP inhibitor is dalcetrapib, torcetrapib, anacetrapib, evacetrapib, obicetrapib, BMS795311, CP-800,569, DLB S-1449, ATH-03, DRL-17822, JNJ-28545595, JNJ-28614872, BAY 19-4789, BAY 38-1315, or BAY 60-5521, or a pharmaceutically acceptable salt of any of the foregoing.

In certain embodiments, the ADCY inhibitor is an ADCY1, ADCY2, ADCY3, ADCY4, ADCY5, ADCY6, ADCY7, ADCY8, ADCY9 or ADCY10 inhibitor.

In certain embodiments, the ADCY inhibitor is SQ22536 (9-(tetrahydro-2-furanyl)-adenine), 2′,5′-dideoxyadenosine, 9-cyclopentyladenine, 2′,5′-dideoxyadenosine 3′-diphosphate, 2′,5′-dideoxyadenosine 3′-monophosphate, MDL-12330A (cis-N-(2-phenylcyclopentyl)azacyclotridece-1-en-2-amine), compounds such as 7,8-dihydro-5(6H)-quinazolinone derivatives disclosed in JP Patent Application No. 2001-153954 (preferably, 2-amino-7-(4-chlorophenyl)-7, 8-dihydro-5 (6H)-quinazolinone, 2-amino-7-(4-methoxyphenyl)-7,8-dihydro-5(6H)-quinazolinone, 2-amino-7-phenyl-7,8-dihydro-5(6H)-quinazolinone, 4.2-amino-7-(2-furanyl)-7,8-dihydro-5(6H)-quinazolinone, and 2-amino-7-(2-thienyl)-7,8-dihydro-5(6H)-quinazolinone), MANT-ATP; MANT-ITP; MANT-GTP; MANT-XTP; MANT-CTP; MANT-UTP; 2′-MANT-3′dATP; 3′-MANT-2′dATP; MANT-ATPγS; MANT-ITPγS; MANT-GTPγS; MANT-UTPγS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-IMP; Bis-Cl-ANT-ATP; Bis-Cl-ANT-ITP; Bis-Br-ANT-ATP; Bis-Br-ANT-ITP; Bis-Pr-ANT-ATP; Bis-Pr-ANT-ITP; Bis-AcNH-ANT-ATP; Bis-AcNH-ANT-ITP; NKY80; vidarabine; 2′,5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; 2′,5′-Dideoxyadenosine 3′-triphosphate tetrasodium salt; or a pharmaceutically acceptable salt of any of the foregoing.

In certain embodiments, the pharmaceutical acceptable carrier or vehicle can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when the CETP inhibitor or ADCY inhibitor is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also comprise minor amounts of wetting or emulsifying agents, or pH buffering agents.

The compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained release formulation; (3) topical application, for example, as a cream, ointment, or a controlled release patch or spray applied to the skin; (4) intravaginally or intrarectally, for example, as a pessary, cream or foam; (5) sublingually; (6) ocularly; (7) transdermally; or (8) nasally.

Compositions of the present invention include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, e.g., from about 5 percent to about 70 percent, or from about 10 percent to about 30 percent.

In certain embodiments, a composition of the present invention comprises an excipient selected from the group consisting of cyclodextrins, celluloses, liposomes, micelle forming agents, e.g., bile acids, and polymeric carriers, e.g., polyesters and polyanhydrides; and a CETP inhibitor or ADCY inhibitor. In certain embodiments, an aforementioned composition renders orally bioavailable a CETP inhibitor or ADCY inhibitor.

Methods of preparing these compositions or compositions include the step of bringing into association a CETP inhibitor or ADCY inhibitor with the carrier and, optionally, one or more accessory ingredients. In general, the compositions are prepared by uniformly and intimately bringing into association a CETP inhibitor or ADCY inhibitor with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a CETP inhibitor or ADCY inhibitor as an active ingredient. A CETP inhibitor or ADCY inhibitormay also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the CETP inhibitor or ADCY inhibitor include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions of the pharmaceutical compositions of the invention for rectal or vaginal administration may be presented as a suppository, which may be prepared by admixing one or both of the CETP inhibitor and ADCY inhibitor with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Compositions of the present invention which are suitable for vaginal administration also include pessaries, tampons, creams, gels, pastes, foams or spray compositions containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a CETP inhibitor or ADCY inhibitor to a subject. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Pharmaceutical compositions of this invention suitable for parenteral administration can comprise a CETP inhibitor, an ADCY inhibitor and one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the composition isotonic with the blood of the intended recipient or suspending or thickening agents.

Examples of suitable aqueous and nonaqueous carriers which may be employed in the compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable compositions are also prepared by entrapping the drug in liposomes or microemulsions which are compatible with body tissue.

When the CETP inhibitor or ADCY inhibitor is administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be administered orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the CETP inhibitor or ADCY inhibitor, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular CETP inhibitor or ADCY inhibitor employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian can readily determine and prescribe the effective amount of the pharmaceutical composition. For example, the physician or veterinarian could start doses of the CETP inhibitor or ADCY inhibitor employed in the pharmaceutical composition at levels lower than that required in order to achieve the desired therapeutic effect and gradually increase the dosage until the desired effect is achieved.

In some embodiments, a suitable daily dose of a CETP inhibitor or an ADCY inhibitor is that amount of the CETP inhibitor or ADCY inhibitor which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms, e.g., one administration per day.

The invention also provides kits useful for treating or preventing a cardiovascular disorder or reducing risk of a cardiovascular event, as described herein. In some embodiments, the kits comprise a CETP inhibitor or an ADCY inhibitor and instructions for its use. In some embodiments, each of the CETP inhibitor and ADCY inhibitor is present in a separate composition. In some embodiments, the CETP inhibitor and ADCY inhibitor are present in the same composition. The invention also provides a CETP inhibitor and an ADCY inhibitor as described herein and compositions comprising an effective amount of a CETP inhibitor and an ADCY inhibitor as described herein for use in the methods described herein.

### EXAMPLES

### Example 1

### Generation of Adyc9 Gene-Trapped Mice

Adyc9 gene-trapped (Adcy9^{Gt/Gt},)mice were generated initially from Lexicon Pharmaceuticals (Zambrowicz et al., Proc Natl Acad Sci USA. (2003)100:14109-14). Cryo-preserved sperm from the B6; 129S5-Adcy9^{Gt(neo)159Lex}/Mmucd strain was imported from the Mutant Mouse Regional Resource Center (MMRRC) and used for in vitro fertilization and oviduct transfer at the Institute for Research in Immunology and Cancer (IRIC) animal facility in a specific-pathogen-free area and their breeding and reproduction was approved by the University de Montréal Deontology Committee on Animal Experimentation. MaxBax accelerated backcrossing and genotyping (Charles River, Sherbrooke, Canada) was performed to achieve >98.6% C57BL/6J genetic background. Procedures involving mice were performed at the Montreal Heart Institute (MHI) Research Center and approved by the local ethics committee for animal research in accordance with the Canadian Council on Animal Care guidelines. Adcy9^{Gt/Gt} mice were bred by mating Adcy9^{WT/Gt} animals. Mice hemizygous for transgenic human CETP minigene (CETP^{WT}) were obtained from Jackson laboratory (Bar Harbor, Me.) and were on 100% C57BL/6J genetic background. To obtain mice that were transgenic for CETP and with homozygous inactivation for Adcy9 (CETP^{Gt}), CETP^{WT} mice were first crossed with Adcy9^{WT/Gt} animals, then mice transgenic for CETP and heterozygote for Adcy9^{WT/Gt} were crossed with Adcy9^{Gt/Gt} or Adcy9^{WT/Gt} animals. All mice were under a C57BL/6J background, males and aged from 8 to 12 weeks. Blood collection was performed after 4 hours of fasting.

### Example 2

### Atherosclerosis Model

Wild-type (WT) and Adcy9^{Gt/Gt} male mice (8 to 12-week-old) were injected with a single dose of an AAV8 viral vector expressing gain-of-function Pcsk9^{D377Y} (AAV8-Pcsk9^{D377Y}, 6.5×10¹¹ gene copies) as previously described. (Roche-Molina et al., Arterioscler Thromb Vasc Biol. (2015) 35:50-59) A control group was injected with a saline solution in preliminary experiments to confirm the effect of the AAV8-Pcsk9^{D377Y} infection on LDL receptor expression. Starting one week after AAV8-Pcsk9^{D377Y} injection, mice were fed for 16 weeks with a chow-based (Purina 5015) atherogenic diet (TD, 150545, Envigo, Madison, Wis.) enriched with 0.75% cholesterol and having the following caloric composition: proteins 20.4%, carbohydrates 42.7% and fats 36.9% (3.9 kcal/g). Food and water were available ad libitum. After the sacrifice, atherosclerosis development was investigated in the whole aorta, aortic root and brachiocephalic artery by an experienced observer blinded to the mouse genotype.

### Example 3

### Adcy9 Inactivation Protects Against Atherosclerosis

Quantification of Adcy9 mRNA expression by reverse transcription-quantitative PCR (RT-qPCR)

Mice total RNA were extracted from heart ventricles using RNeasy isolation kit (QIAGEN, Toronto, ON, Canada) with DNase I procedure according to the manufacturer's protocol. cDNA was synthesized with components from High-Capacity cDNA Reverse transcription Kits without inhibitor (Applied Biosystems #4368814, Foster City, Calif.) and with the use of MultiScribe™ Reverse Transcriptase, according to the manufacturer's procedures. RNA quantification and quality were assessed using Agilent RNA 6000 Nano Kit for Bioanalyzer 2100 System (Agilent Technologies, Santa Clara, Calif.). Primers for Adcy9 were designed using the Beacon designer software v.8 (Premier Biosoft) and obtained from IDT (Coralville, Iowa). Gapdh was used as the reference gene for normalization. The quantitative PCR (qPCR) was performed with SYBR-Green reaction mix (BioRad, Hercules, Calif.). The qPCR conditions consisted of an initial denaturation at 95° C. for 3 minutes followed with 40 cycles of amplification, each cycle consisting of 95° C. for 10 seconds and 60° C. for 30 seconds.

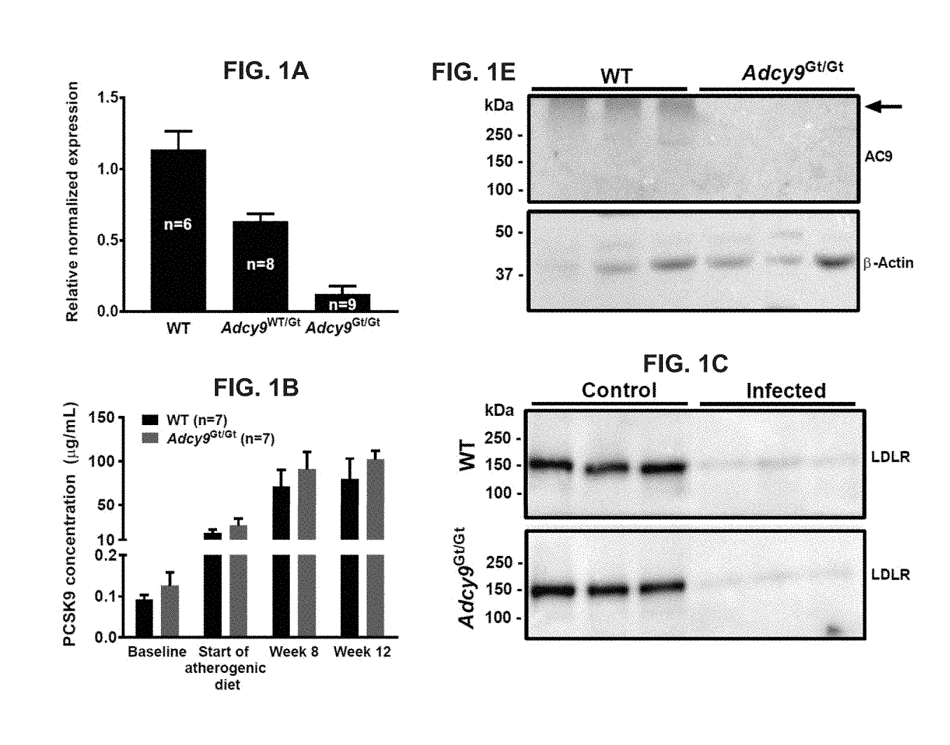

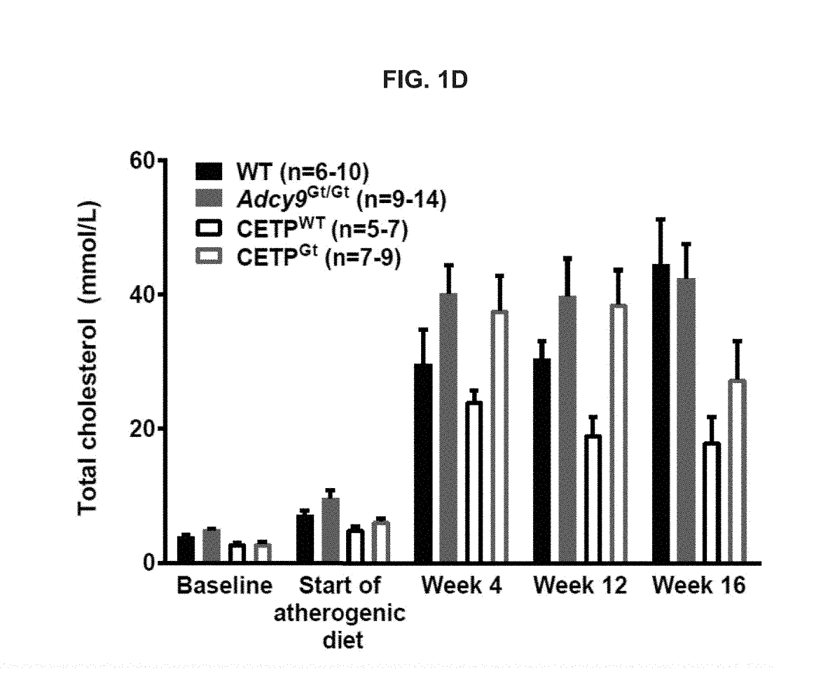

The effect of the Adcy9^{Gt} modified target locus on Adcy9 expression in Adcy9^{Gt/Gt} mice was characterized. Using RT-qPCR, it was demonstrated that Adcy9 mRNA expression in the heart was decreased by 50% in Adcy9^{WT/Gt} mice and by at least 90% in Adcy9^{Gt/Gt} mice compared to WT (FIG. 1A). In the skeletal muscle, where the ADCY9 gene has been reported to be highly expressed, it was observed that protein expression was abolished in Adcy9^{Gt/Gt} mice compared to WT (FIG. 1E). Then, WT and Adcy9^{Gt/Gt} mice were infected with AAV8-Pcsk9^{D377Y} to induce hypercholesterolemia. One week after infection and before the start of the atherogenic diet, plasma PCSK9 concentration was dramatically increased from 0.1 μg/mL to 17.9±3.5 μg/mL in WT and 26.6±7.5 μg/mL in Adcy9^{Gt/Gt} mice. PCSK9 concentrations increased to 71.0±19.1 μg/mL in WT and 90.8±20.1 μg/mL in Adcy9^{Gt/Gt} mice after 8 weeks of atherogenic diet, where they stayed almost stable at 12 weeks (FIG. 1B). PCSK9^{D377Y} induced a reduction of 90% in hepatic LDL receptor expression that was similar in WT and Adcy9^{Gt/Gt} (FIG. 1C).

Determination of plasma cholesterol concentration, lipoprotein profile and plasma PCSK9

Blood samples were collected in EDTA-coated tubes and plasma was separated by centrifugation (2000×g, 10 minutes) and conserved at −80° C. until analysis. Total cholesterol was quantified by colorimetric enzymatic assay (Wako Diagnostics, 999-02601, Richmond, Va.) and measured at 600 nm in 96-well black microplates with flat bottom according to a micro-method used in our laboratory. To determine the lipoprotein profile, blood samples were pooled from 5 mice per group and a fast protein liquid chromatography (FPLC) size fractionation on Sepharose was performed by a specialized lipidomics core facility (University of Alberta, Edmonton, AB). PCSK9 concentration in plasma was determined using an ELISA kit targeting mouse PCSK9 (Cyclex Co, #CY-8078, Japan).

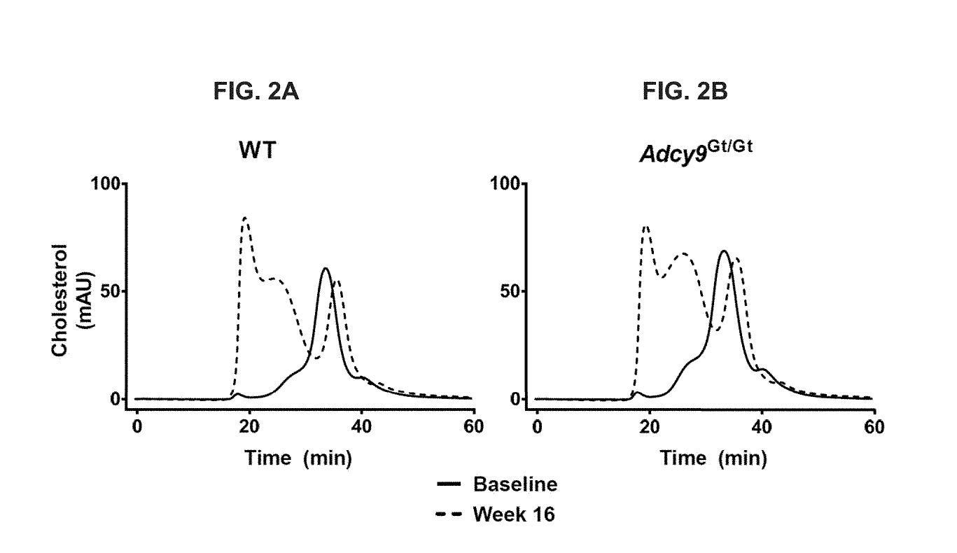

Plasma cholesterol levels rose from 3.8±0.3 mM in WT and 4.9±0.2 mM in Adcy9^{Gt/Gt to} 44.5±6.7 mM and 42.4±5.1 mM (P=NS) at 16 weeks (FIG. 1D). Visualization of lipoprotein profile by chromatography showed that WT and Adcy9^{Gt/Gt} mice had a similar profile rich in HDL at baseline. PCSK9^{D377Y} and the hypercholesterolemic diet induced an atherogenic redistribution of cholesterol out of HDL particles and into VLDL and LDL particles in both types of mice (FIG. 2A and FIG. 2B).

En Face Quantification of Aortic Atherosclerotic Lesions

Atherosclerosis lesions were quantified on whole aorta dissected en face. Aortas were fixed in 4% paraformaldehyde (PFA) overnight and then stained with a 0.7% solution of Oil Red O Sigma-Aldrich) for 1 hour and then counterstained with Nuclear Fast Green at 0.05% (Sigma-Aldrich, F7258) to optimize contrast. Images were captured using a Leica Microsystem (Concord, ON, Canada) stereomicroscope with digital camera (MC170 HD, Leica Microsystem). Atherosclerotic lesions were quantified from the aortic root down to the iliac artery bifurcation using Image-Pro Premier 9.2 (Media Cybernetics, Inc, Rockville, Md.). Percent atherosclerotic lesion area was expressed as the percentage of the intima area presenting Oil Red O staining.

Atherosclerotic lesion cross-sectional quantification in the aortic root

The base of the heart was embedded in OCT compound and sliced in 10 μm sections before fixation with 10% cold formalin for five minutes and then rinsed twice with deionized water. Slides were immersed twice in 100% propylene glycol for five minutes. Aortic roots were stained with a 0.7% Oil Red O solution for 48 hours at room temperature. Afterwards, slides were successively immersed in 100% propylene glycol, 85% propylene glycol solution and deionized water baths, using agitation. Slides were counterstained with a hematoxylin solution for a few seconds, rinsed and then mounted using an aqueous medium. Atherosclerosis lesions were quantified at regular distances from the base of the aortic sinuses to 500 μm more distally in the aortic root. Images were obtained by microscope and quantified by Image-Pro Premier 9.2.

Atherosclerotic Lesion Quantification in the Brachiocephalic Artery

The presence of plaques, fibrin, red blood cells and break in the plaque cap was determined in the brachiocephalic artery. All solutions used for staining were from EMS (Hatfield, Pa.) except for phosphotungstic acid that was from Sigma-Aldrich. Six micrometer-thick sections (6 μm), taken from paraffin-embedded brachiocephalic artery, were stained according to the Carstairs' method with modification as described previously'. After rehydration, sections were incubated in 5% ferric ammonium sulfate solution for 5 minutes and rinsed with tap water. Sections were then stained with Mayer hematoxylin solution for 5 minutes and rinsed with tap water. Additional stainings were performed with picric acid orange G for 1 hour and Ponceau-fuchsin for 5 minutes before rinsing with distilled water for each staining. Muscle tissue was differentiated from other structures with 1% phosphotungstic acid for 3 minutes before rinsing with distilled water. Final staining for collagen was performed with Anilin Blue (2.5%, 2 minutes) before dehydration clearing with xylene and mounting in Permount (Thermo Fisher Scientific, Toronto, ON, Canada). The sections were scored for presence of plaques, fibrin deposition at the surface of and in the plaque, and breaks in the plaque cap. To be scored positively, a mouse needed to show the feature of interest in two sections separated by 48 μm. Plaque size was scored according to the percentage of cross-sectional area occupied by plaque (scores of 0: <50%, 1: 50-75%, and 2: >75%).

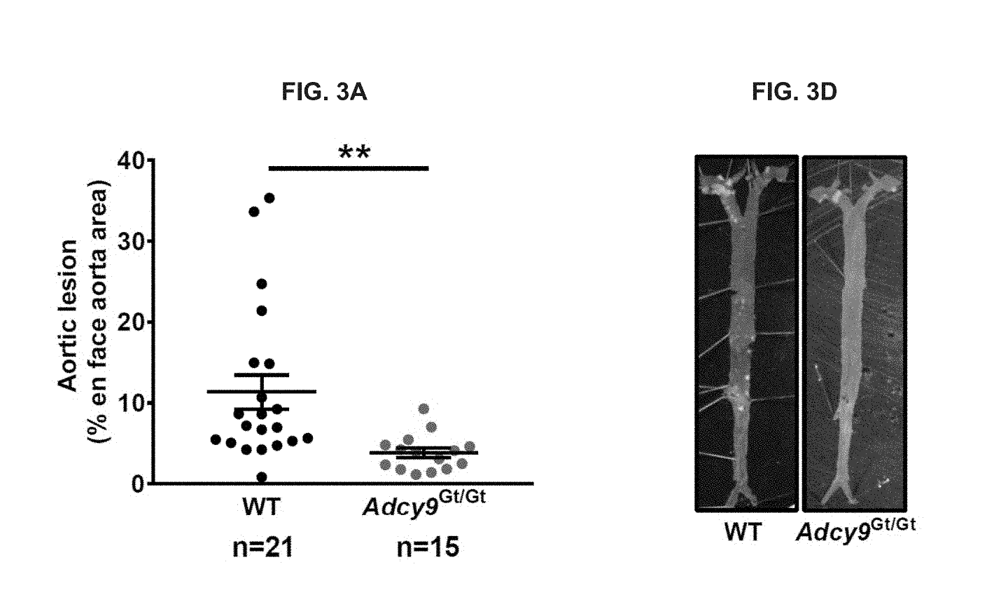

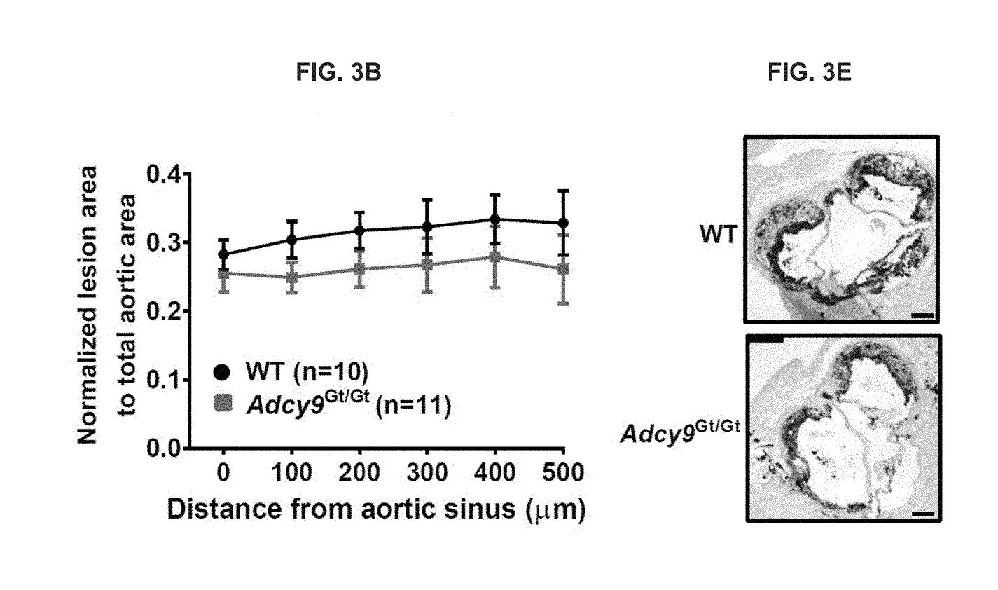

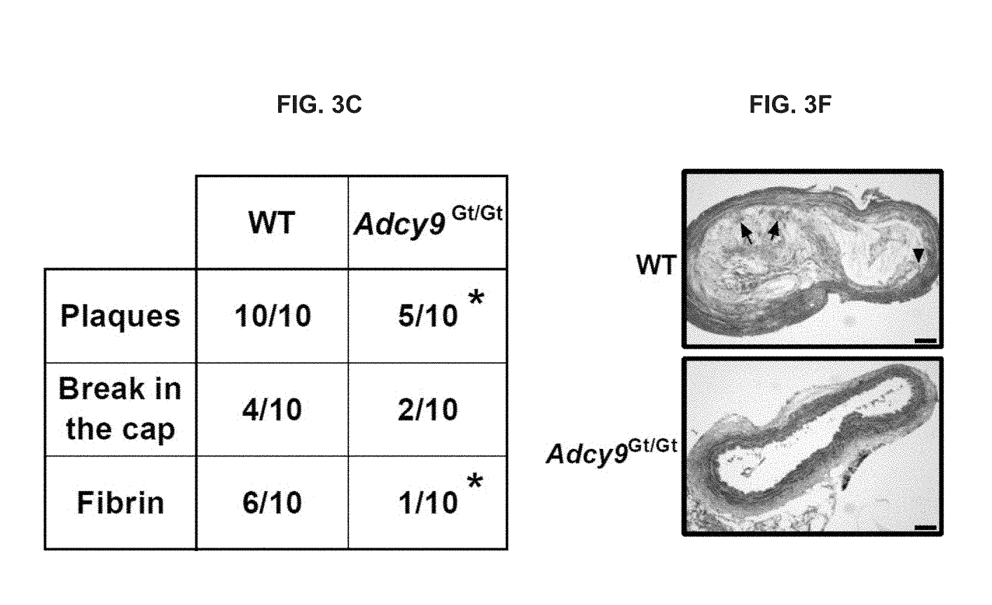

At sacrifice, aortas showed 11.3±2.1% of the surface of the intima covered with atherosclerotic lesions in WT mice compared to 3.8±0.6% in Adcy9^{Gt/Gt} mice (P<0.01, FIG. 3A and FIG. 3D), a reduction of 65%. Cross-sectional quantification showed a numerical reduction of plaque area all along the aortic root by Adcy9 inactivation (p=0.07 at 500 μm, FIG. 3B and FIG. 3E). Brachiocephalic arteries are the site in mice where plaques are more complex and prone to rupture (Rosenfeld et al., Arterioscler Thromb Vasc Biol. (2000) 20:2587-92); FIG. 3C shows that all WT animals had plaques in the brachiocephalic arteries in contrast to Adcy9^{Gt/Gt} mice (100%, versus 50%, P<0.05). The plaques in WT mice were also larger and presented more frequently fibrin deposits at their surface (P<0.05) and cap ruptures compared to Adcy9^{Gt/Gt} mice (FIG. 3F).

### Example 4

### Adcy9 Inactivation Reduces CD68-Positive Macrophage (Foam Cell) Accumulation and their Proliferation in Atherosclerotic Lesions

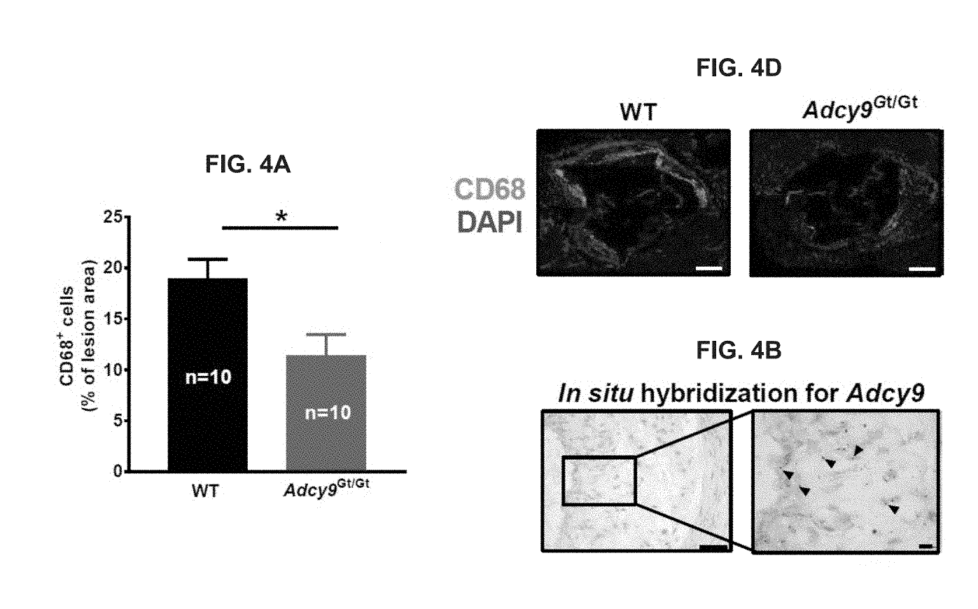

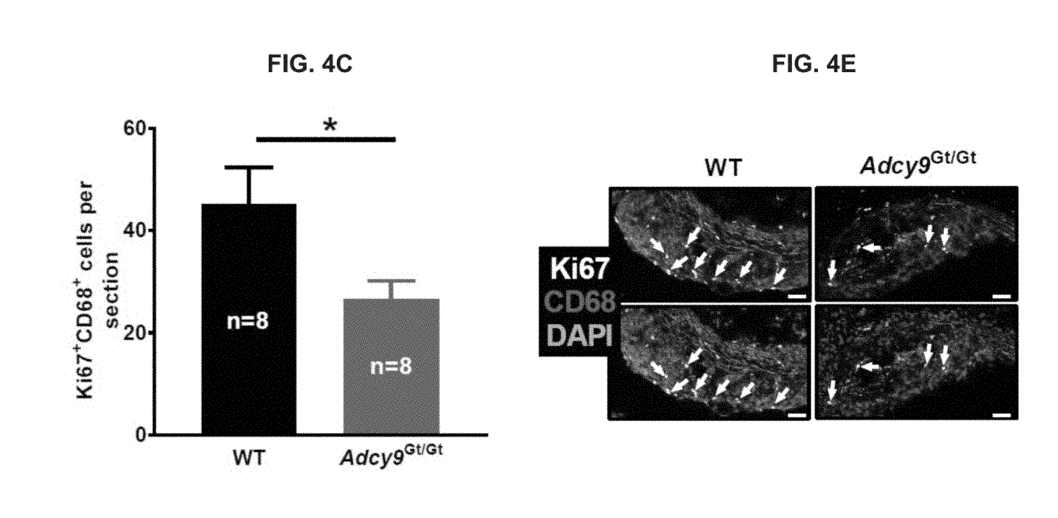

Immunofluorescent detection of CD68-positive macrophages (foam cells) and their proliferation in the aortic root (FIG. 4A and FIG. 4D)

The base of the heart was embedded in OCT compound and 10 μm sections were cut with a cryostat. Slides were incubated in PBS to remove the OCT and then immersed in 4% PFA (pH 7.4) for 20 minutes. After fixation, slides were washed in PBS. Permeabilization, blocking and antibody incubation were performed in a Tris solution (in mM): Tris-base 50, NaCl 150, BSA 1%, Triton X-100 0.4% and fetal bovine serum 20% at pH 7.4. Rat antibody against CD68 was diluted 1/200 (BioRad, #MCA19557) and rabbit antibody against Ki67 was used at 1 μg/mL (Abcam, #ab15580). For determination of CD68-positive cell accumulation in the atherosclerotic lesions, CD68 was visualized using goat anti-rat antibody labeled with Alexa-647 (Thermo Fisher Scientific, #cat A-21247). For co-staining with Ki67, CD68 was visualized with goat anti-rat antibody labeled with Alexa-568 (Thermo Fisher Scientific, #A11077) and Ki67 was visualized with a goat anti-rabbit antibody labeled with Alexa-647 (Thermo Fisher Scientific, #A-27040). The nuclei were counterstained with DAPI. Images were acquired with a confocal microscope (LSM 710, Zeiss, Peabody, CA). Imaging quantifications were performed with Image-Pro Premier 9.2 from three sections of the aortic root. Accumulation of CD68-positive foam cells was quantified as percent of the total lesion area from three aortic root sections. Cells were considered positive for Ki67 if the staining colocalized with DAPI. To evaluate proliferation of CD68-positive foam cells, double positive Ki67/CD68 cells were counted.

CD68-positive foam cells, a key constituent of atherosclerotic plaques, represented 19.0±1.9% and 11.4±2.1% of the lesion area in WT and Adcy9^{Gt/Gt} mice, respectively (P<0.05, FIG. 4A).

In situ hybridization showed Adcy9 expression in atherosclerotic lesions at sites of foam cell accumulation (FIG. 4B). In situ hybridization was performed according to the manufacturer's recommended procedure (Advanced Cell diagnostic, Hayward, CA). Staining was performed on 6 μm-thick sections of paraffin-embedded femoral artery or aortic root. Sections were counterstained with hematoxylin. It was observed that the signal for Adcy9 was specific by comparing tissue sections from Adcy9^{Gt/Gt} and WT mice (data not shown). Adcy9 probe was Mm-Adcy9 targeting nucleotides 1522-2502 of NM_009624.3. Probe for the bacterial gene dapB was used as a negative control.

Immunofluorescence detection of Ki67 revealed that proliferation of CD68-positive foam cells was significantly decreased in Adcy9^{Gt/Gt} compared to WT (P<0.05, FIG. 4C and FIG. 4E).

### Example 5

### Adcy9 Inactivation Potentiates Endothelial Function

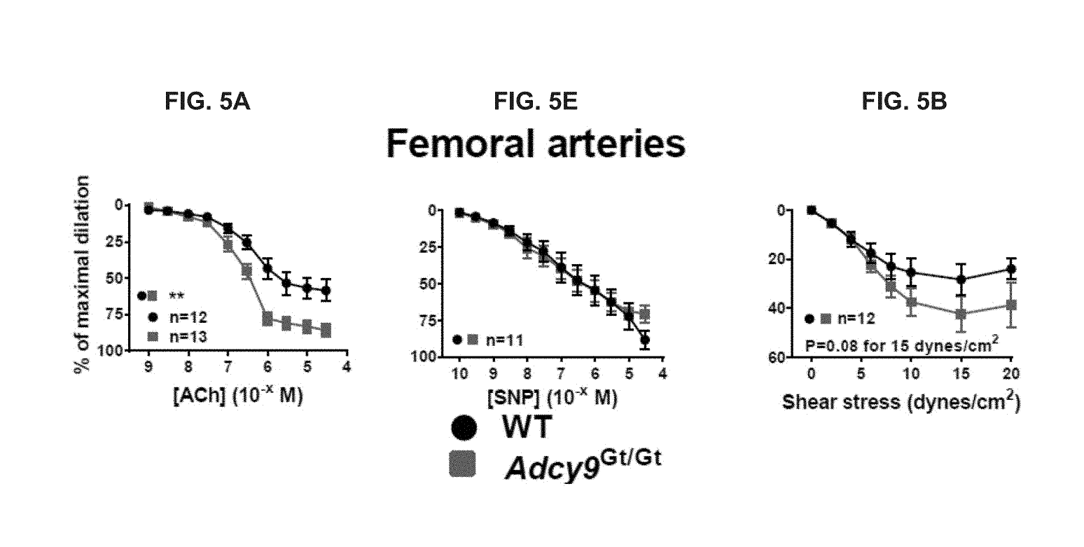

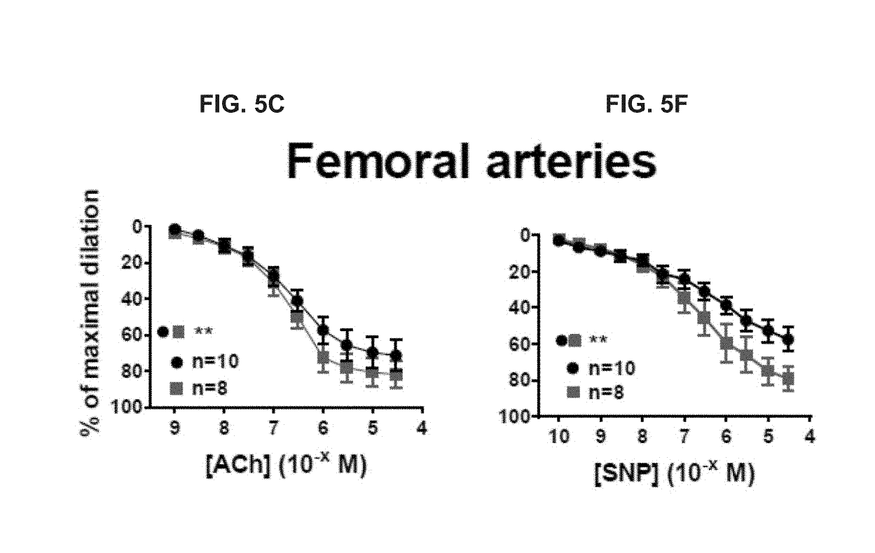

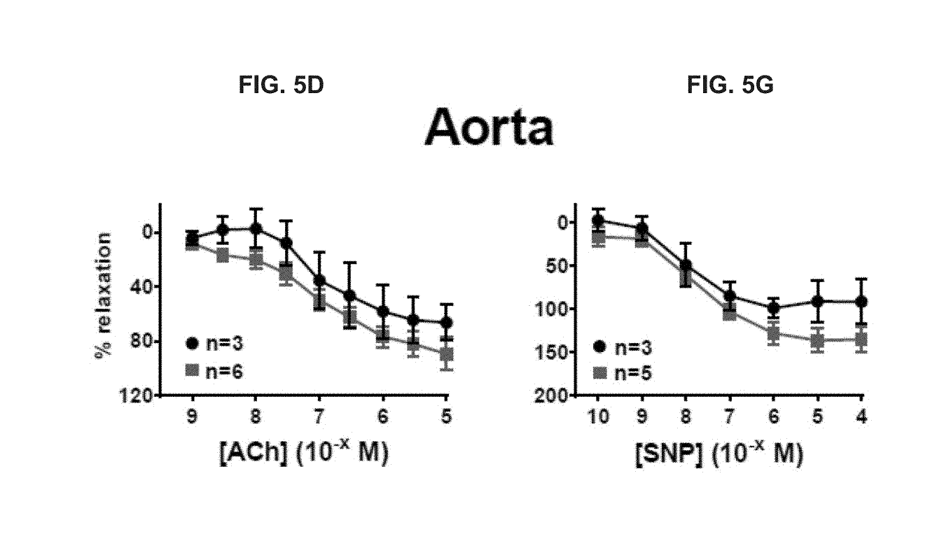

Endothelial-dependent vasodilatation to ACh in untreated animals (no AAV8-Pcsk9^{D377Y} and no atherogenic diet) was potentiated in the femoral arteries of Adcy9^{Gt/Gt} mice compared to WT (P<0.01, FIG. 5A); in contrast, endothelial-independent vasodilation to the nitric oxide donor SNP was similar in both groups (FIG. 5E). Adcy9 inactivation also potentiated endothelial-dependent vasodilatation in response to increased shear stress (15 dynes/cm²) compared to WT (42.3±7.1% versus 28.2±6.3%, P=0.08, FIG. 5B). There was no difference detected between untreated mice (no AAV8-Pcsk9^{D377Y} and no atherogenic diet) when the aorta was evaluated. In femoral arteries from atherosclerotic animals (AAV8-Pcsk9^{D377Y} and atherogenic diet) both endothelial-dependent (ACh-induced) and endothelial-independent vasodilatation were increased in Adcy9^{Gt/Gt} mice compared to WT (P<0.05, FIG. 5C and FIG. 5F). This suggests that sensitivity of vascular smooth muscle cells to NO might be increased by Adcy9 inactivation. Similar benefits were observed in the aorta of animals treated with the AAV8-Pcsk9^{D377Y} and atherogenic diet when comparing Adcy9^{Gt/Gt} to WT mice (FIG. 5D and FIG. 5G).

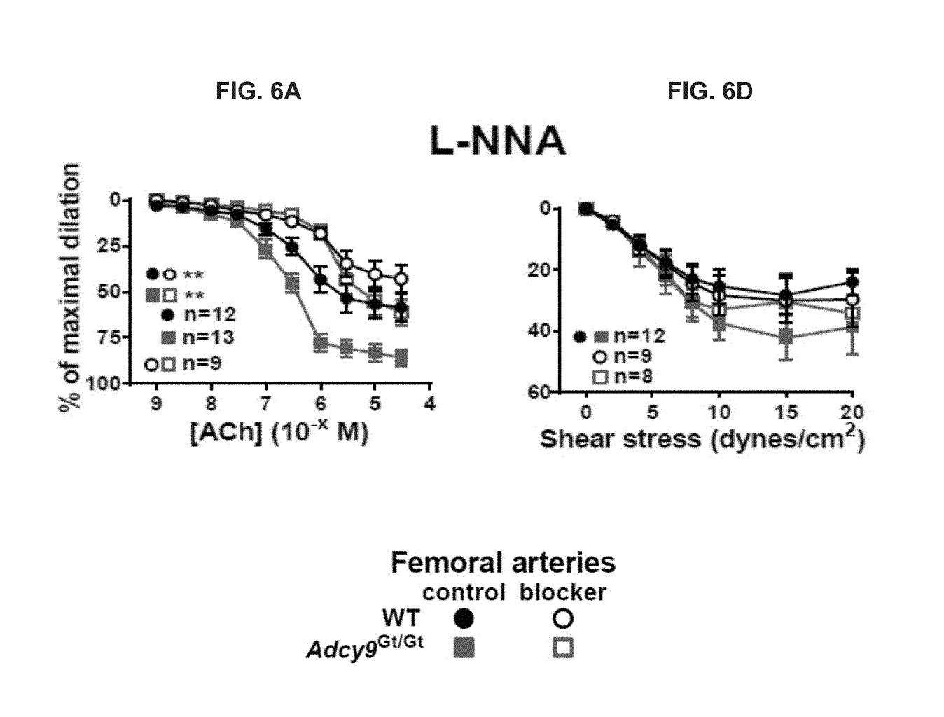

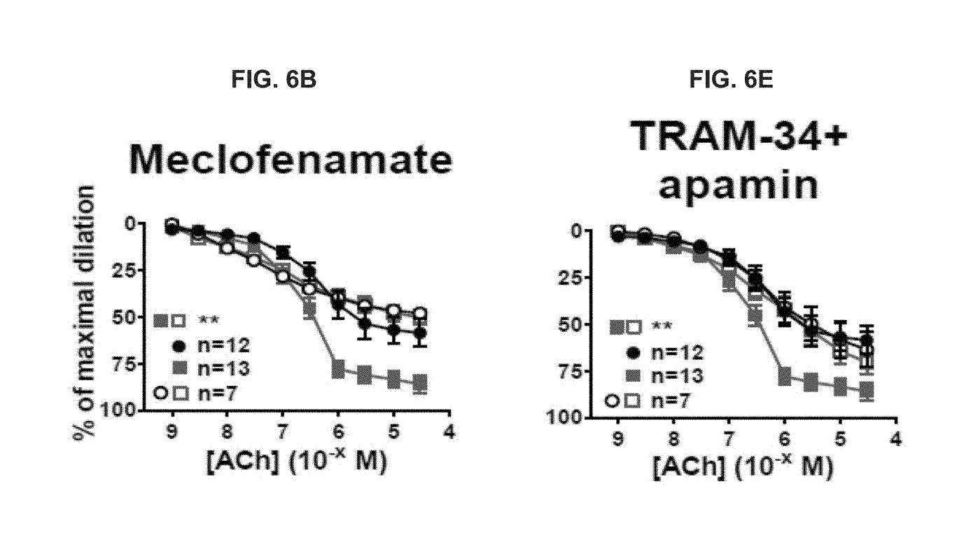

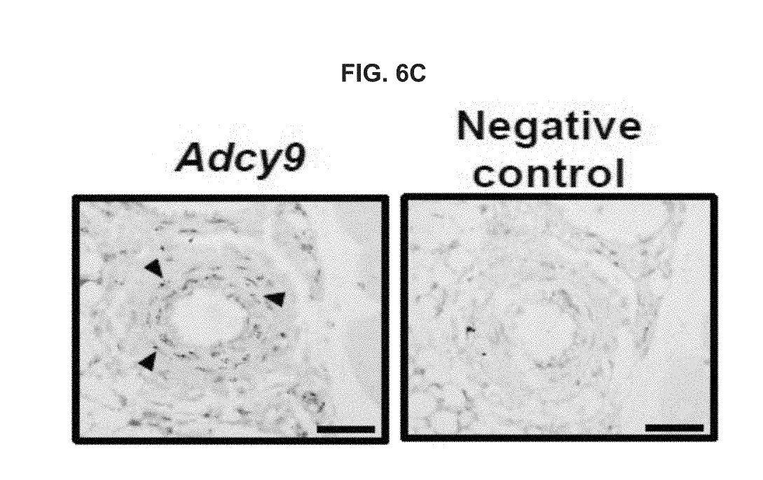

Selective pharmacological blockade of the signaling pathways responsible for endothelial-dependent vasodilatation (Vanhoutte et al., Acta Physiol (Oxf). (2017) 219:22-96) was then used to identify the mechanism responsible for the improvement observed in Adcy9^{Gt/Gt} mice. The nitric oxide synthase blocker L-NNA inhibited significantly ACh-induced vasodilatation both in WT and Adcy9^{Gt/Gt} mice (P<0.01), but flow-mediated vasodilatation was numerically reduced only in Adcy9^{Gt/Gt} mice (FIG. 6A and FIG. 6D). In contrast, the cyclooxygenase (responsible for production of prostacyclin) blocker meclofenamate or the cocktail of endothelial-dependent hyperpolarization blockers (TRAM-34 and apamin), inhibited ACh-induced endothelial-dependent vasodilatation in Adcy9^{Gt/Gt} mice (P<0.01 for both pathways, FIG. 6B and FIG. 6E) but not in WT mice. Overall, these pharmacological results demonstrate that the effect of Adcy9 inactivation on endothelial function is dependent on all three endothelial cell signaling pathways.

Adcy9^{Gt/Gt} mice are systemically inactivated for Adcy9 making it difficult to identify its role in vascular tissue that could be associated with the observed potentiated endothelial function. Therefore, Adcy9 mRNA expression was studied by in situ hybridization on histological sections and was demonstrated in the femoral artery wall. Specificity of the Adcy9 mRNA signal was confirmed by absence of dots with the negative control probe (FIG. 6C).

### Example 6

### Adcy9 Inactivation Increases Body Weight and Adipose Tissue Volume

Body weight was measured every week from the week of infection with AAV8-Pcsk9^{D377Y} and for a total of 16 weeks. Feed efficiency, the ratio of the weight gain to the caloric intake (g/100kcal), was calculated for a total of 8 weeks during the atherogenic diet.

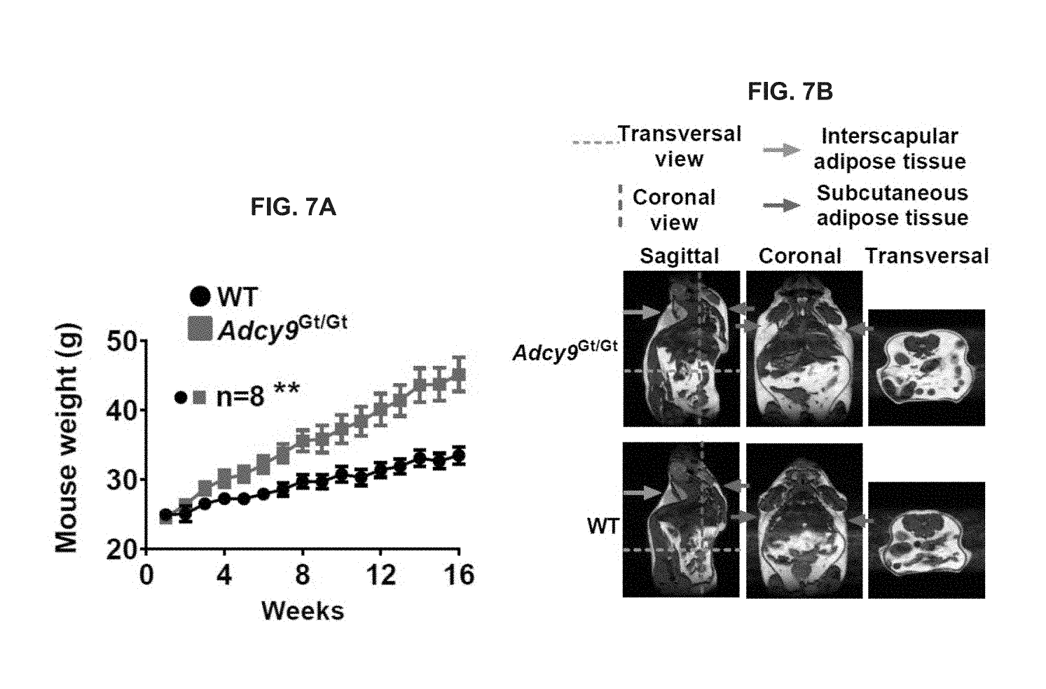

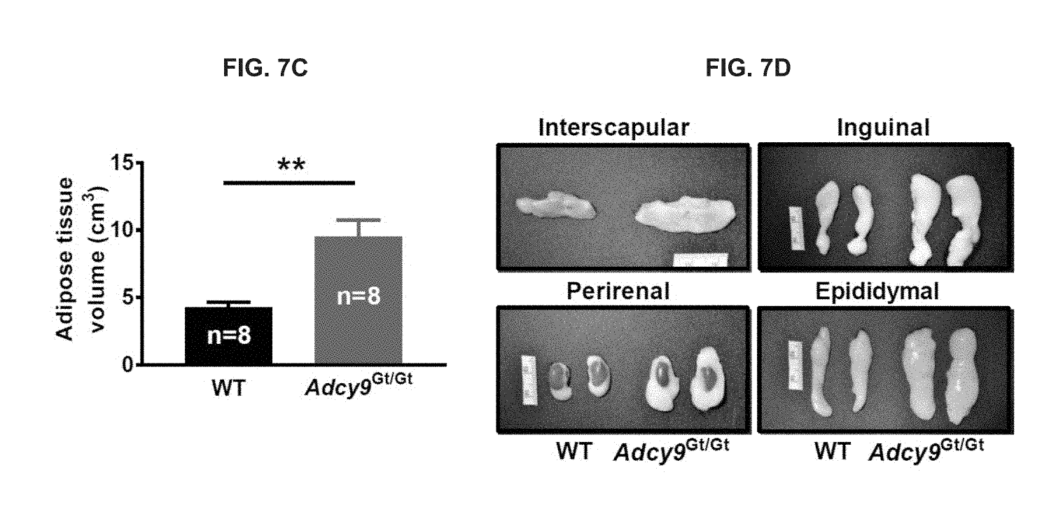

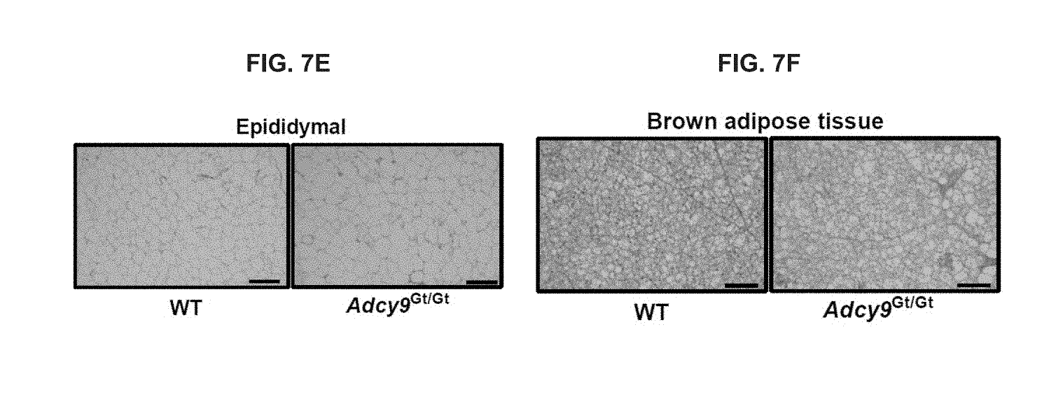

Adcy9^{Gt/Gt} mice gained more weight than WT animals during the 16 weeks of atherogenic diet, to reach weights of 45.1±2.4 g and 33.5±1.2 g respectively (P<0.01, FIG. 7A). MRI performed at week 14 showed that Adcy9^{Gt/Gt} mice presented more adipose tissue than WT (FIG. 7B), with a doubling of total body adipose tissue volume in Adcy9^{Gt/Gt} mice (9.4±1.2 cm³) compared to WT (4.3±0.4 cm³, P<0.01, FIG. 7C). This increase was confirmed by the larger inguinal, perirenal, epididymal and interscapular fat depots (FIG. 7D). Histological sections showed that Adcy9 inactivation resulted in hypertrophic adipocytes in epididymal white adipose tissue and larger lipid droplets in interscapular brown adipose tissue (FIG. 7E and FIG. 7F). No significant change in blood glucose or insulin concentrations was observed in response to the atherogenic diet and increased fat deposits, suggesting that the weight gain in response to Adcy9 inactivation does not modify insulin sensitivity (data not shown).

### Example 7

### Adcy9 Inactivation Modulates Autonomic Nervous System Activity

Telemetry for ECG Recording

Telemetry was used to monitor and record ECG in conscious free moving unanesthetized WT and Adcy9^{Gt/Gt} mice. Mice were equipped with an ECG transmitter (ETA-F10, DSI, St Paul, MN) for mice and electrodes were placed subcutaneously on the abdomen in a conventional lead II position (Brouillette et al., JMol Cell Cardiol. (2007) 43:159-67). Recordings were performed continuously at 1 KHz per channel with IOX (version 2.8.0.17, EMKA Inc) 10 days post-surgery. ECGs were analyzed for heart rate variability (HRV) according to the guide by Thireau et al. (Exp Physiol. (2008) 93:83-94), and with the software ECG Auto (version v3.3.0.25, EMKA Inc). Periods containing 1500 beats for day time (6 AM to 6 PM) and night time (6 PM to 6 AM) were analyzed. Data were analyzed to extract the time domain parameters RR interval and pNN(6), the latter representing the percentage of RR intervals exceeding preceding ones by 6 ms or more and reflects parasympathetic nervous system activity.

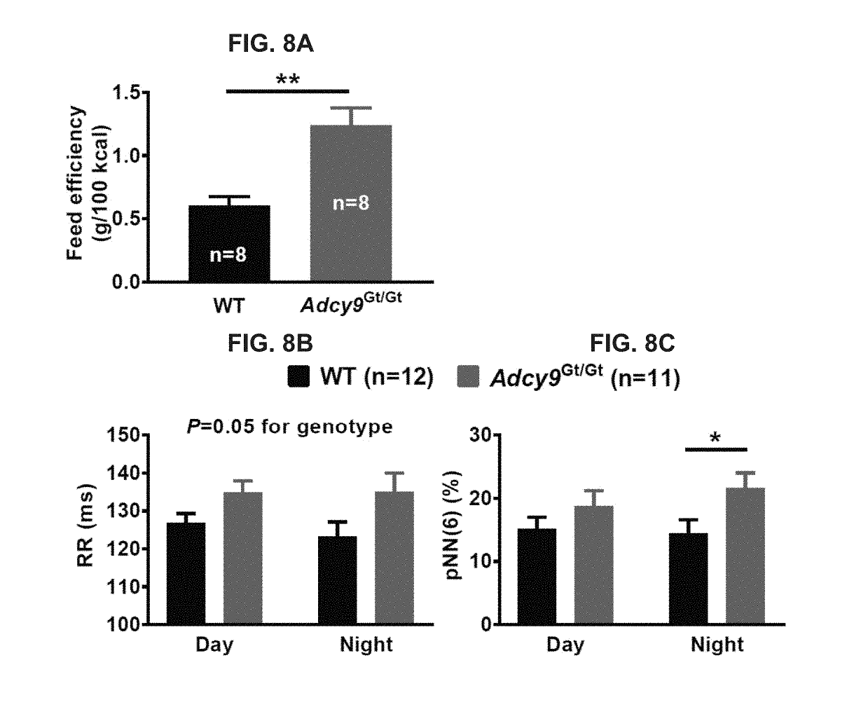

Given the differences in weight gain between Adcy9^{Gt/Gt} and WT mice, feed efficiency defined as the ratio of weight gain over energy intake was evaluated over the same period of time. With the atherogenic diet, Adcy9^{Gt/Gt} mice showed a significant increase in feed efficiency (1.2±0.1 g/100 kCal) compared to WT (0.6±0.1 g/100 kCal, P<0.01, FIG. 8A). This indicates that the control of energetic balance, which is dependent on the autonomic nervous system, is modified by Adcy9 inactivation. Therefore, autonomic nervous system activity was evaluated by measuring heart rate variability by telemetry in WT and Adcy9^{Gt/Gt} mice. The RR interval was increased in Adcy9^{Gt/Gt} mice (135±5 ms) compared to WT mice (123±4 ms, P=0.05, FIG. 8B). Nocturnal heart rate was lower in Adcy9^{Gt/Gt} mice (447±10 bpm) compared to WT mice (475±10 bpm, P=0.06). pNN(6), which represents the percentage of RR intervals exceeding preceding ones by 6 ms or more and reflects parasympathetic nervous system activity'', was increased during the night period in Adcy9^{Gt/Gt} mice (21.6±2.4%) compared to WT (14.5±2.2%, P<0.05, FIG. 8C).

### Example 8

### Adcy9 Inactivation-Induced Atheroprotection is Lost in Mice Expressing CETP

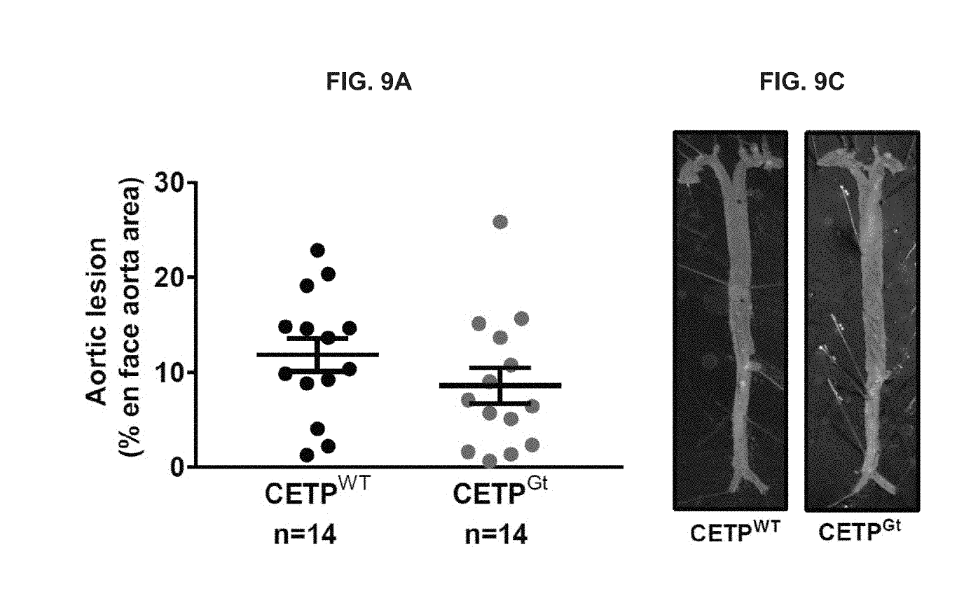

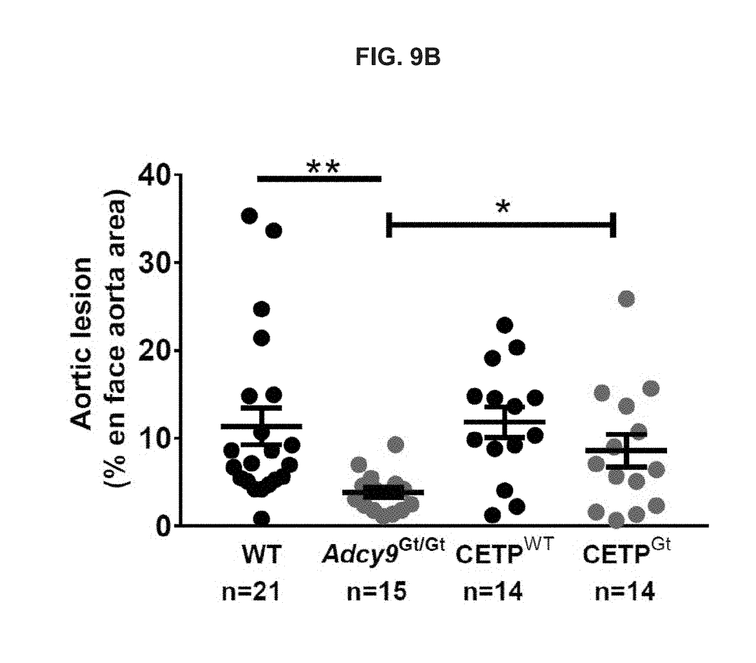

In order to study the interactions between Adcy9 and CETP expression, transgenic mice for the human CETP minigene (CETPW^{T}) were crossed with Adcy9^{Gt/Gt} mice to obtain animals with CETP and inactivated for Adcy9 (CETP^{Gt}). Both types of mice (CETP^{WT} and CETP^{Gt}) were then subjected to the previously described atherogenic protocol (involving AAV8-Pcsk9^{D377Y} injection and atherogenic diet). CETP^{WT} and CETP^{Gt} mice developed similar atherosclerotic lesions that covered 11.8±1.8% and 8.6±1.9% of the intima area, respectively (P=NS, FIG. 9A and FIG. 9C). FIG. 9B compares percentage of lesion area in the whole aorta in WT, Adcy9^{Gt/Gt,} CETP^{WT} and CETP^{Gt} to emphasize the absence of atheroprotection in CETP^{Gt} mice compared to Adcy9^{Gt/Gt} mice (P<0.05). These results indicate that Adcy9 does not significantly affect atherosclerosis development in presence of CETP.

### Example 9

### Adcy9 Inactivation-Induced Endothelial Function Protection is Lost in Mice Expressing CETP

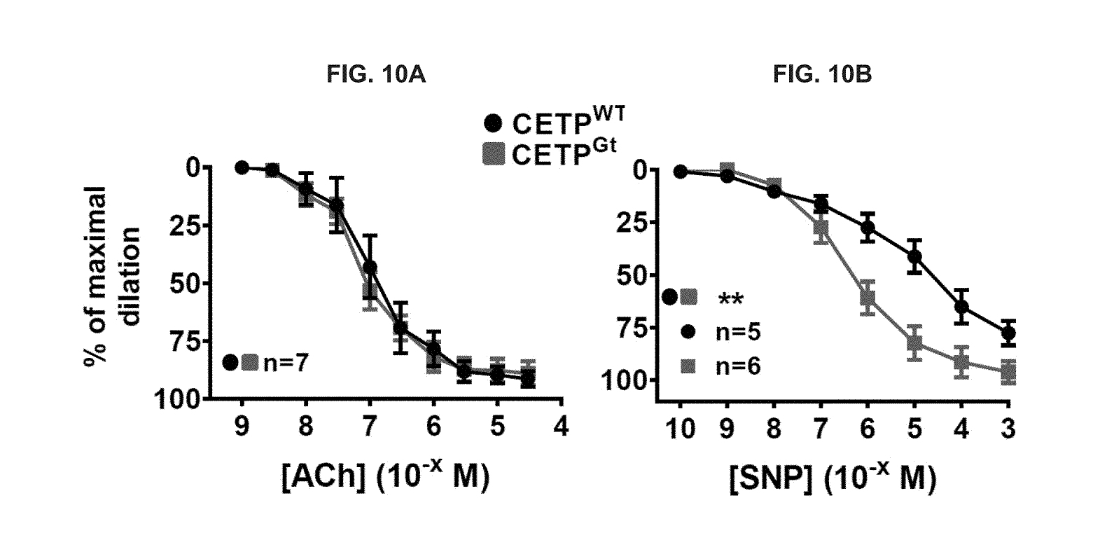

Given the loss ofAdcy9 inactivation-induced atheroprotection when the CETP gene is introduced, vasodilatation to ACh and SNP in femoral arteries from atherosclerotic CETP^{WT} and CETPGt (FIG. 10A and FIG. 10B) were studied. Adcy9 inactivation no longer modified endothelial-dependent vasodilatation in response to ACh when CETP was present (FIG. 10A). However, SNP-induced vasodilatation remained potentiated by Adcy9 inactivation in mice with CETP (P<0.01 for CETP^{Gt} versus CETP^{WT}, FIG. 10B). These results suggest that the atheroprotective effect of Adcy9 inactivation in the absence of CETP is associated with their role in the regulation of endothelial function.

### Example 10

### Adcy9 Inactivation-Induced Gains in Body Weight and Adipose Tissue Volume are Lost in Mice Expressing CETP

Quantification of Whole Body Adipose Tissue Volume by MRI

Whole mouse MRI was performed in the prone position on a 30 cm 7T horizontal MR scanner (Agilent, Palo Alto, Calif.), with a 12 cm inner diameter gradient coil insert, gradient strength 600 mT/m, rise-time 130 .is and an Agilent quadrature transmit/receive birdcage coil with an internal diameter of 38 mm. A pressure transducer for respiratory monitoring and gating (used for shimming) was placed under the abdomen near the sternum. Anesthesia was maintained with 2.0-3.0% isoflurane in pure oxygen to target a respiration rate between 80 and 120 BPM while heart rate and oxygen saturation were monitored with a pulse oximeter on the right hindpaw and body temperature was maintained at 37.0° C. with a rectal probe and a warm air fan (SA Instruments, Stony Brook, N.Y.). A 3D TFISP sequence was used to image the whole mouse (except the tail), with isotropic voxels and FOV 70×36×36 mm, matrix size 256×192×192, resolution 270×190×190 μm, an asymmetric 500 μs pulse with 30 degrees flip angle, TR/TE=4.8/2.4 ms, 83 kHz acquisition bandwidth, 8 frequency-shifted scans reconstructed by square-root-of-sum-of-squares, for an acquisition time of 24 minutes. Mouse fat was manually quantified using ITK-SNAP (Yushkevich et al., Neuroimage. (2006) 31:1116-28).

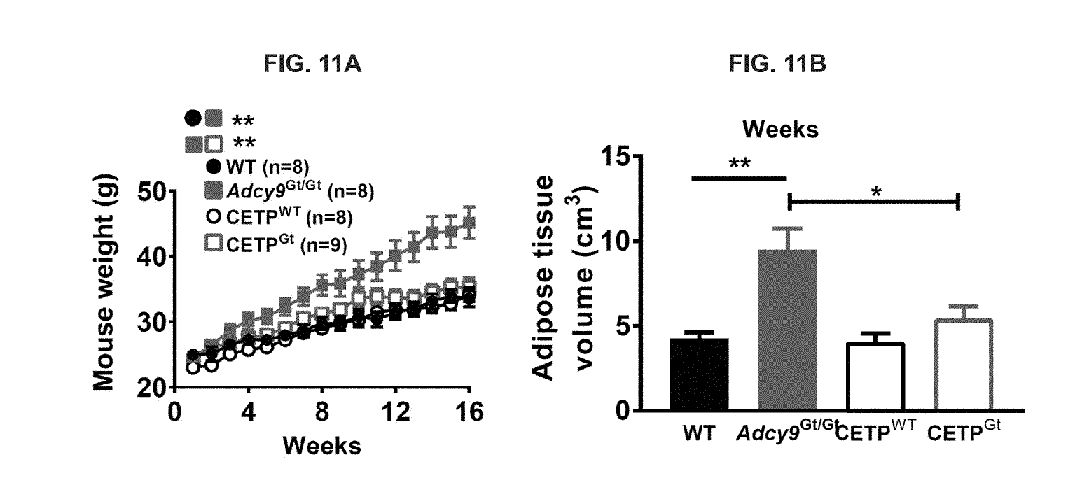

To further study the interactions between Adcy9 and CETP, the effect of Adcy9 inactivation on body weight change in mice expressing CETP during the atherogenic treatment was investigated. CETP^{WT} and CETP^{Gt} mice had similar progression of body weight upon atherogenic treatment (FIG. 11A). At sacrifice, their weights (CETP^{WT}: 34.1±1.1 g; CETP^{Gt}: 35.6±1.3 g) were similar to that of WT animals (33.5±1.2 g) and significantly lower than the weight of Adcy9^{Gt/Gt} mice (45.1±2.4 g, P<0.01, FIG. 11A). At week 14, adipose tissue volumes on MRI were similar in CETP^{WT} (4.1±0.5 cm³) and CETP^{Gt} mice (5.4±0.7 cm³, FIG. 11B). These results demonstrate that Adcy9 and CETP also interact in the regulation of weight and adiposity.

The entire disclosure of each of the patent documents and scientific publications referred to herein is incorporated by reference for all purposes.

1. A method for treating or preventing a cardiovascular disorder, comprising administering to a subject in need thereof an effective amount of:
a) a CETP inhibitor; and
b) an IsIDCY inhibitor.

2. (canceled)

3. The method of claim 1, wherein the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

4. The method of claim 1, wherein the CETP inhibitor is
S-[2-(1-isopentylc:ₛ7clohexa.necarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonyiamino)phenyl]chlorothioacetate;
S-[2-(1-isopentylcyclohex anecarbonytami no)phenyl ]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S-[4,5-dichloro-2-(1-isopentylcyclopentanecarbonylamino)-phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylaminophenyl monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-ditnethylthiopropionate;
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)-phenyl]2,2-dimethylthiopropionate;
S-[4,5-diehloro-2-(1-cyclopropylmethylcyclohexaneeathonyl amino)phenyl]2,2-dimethylthiopropionate;
S-[4,5-dichloro-2-(1-cyclohexylmethylcyclohexanecarbonylamino)phenyl]2,2-dimethylthiopropionate;
S-[4,5-dichloro-2-(1-isopropylcyclohexanecarbonylamino)-phenyl]2,2-dimethylthiopropionate;
S-[4.5-dichloro-2-(1-isopentyl cycloheptanecarbonylamino)-phenyl]2,2-dimethylthiopropionate;
S-[4,5-dichloro-2-(1-isopentylcyclobutanecarbonylamino)-phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethytthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate;
bi s[4,5-dichloro-2-(1-isopentylcyclohexanecarbonylamino)-phenyl]disulfide;
2-tetrahydrofurylmethyl 2-(1-isopentylcyclohexanecarbonylamino)phenyl disulfide;
N-(2-mercaptophenyl)-1-ethylcyclohexanecarboxamide;
N-(2-mercaptophenyI)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate;
bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methyl thiopropinate;
S-[2-(1-isobutylcyclohexanecarbonylamino)phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]chlorophenoxythioacetate;
S-[2-(1-isobutylcyclohexanecarbonylamino)phenyl]4-chlorophenoxythioacetate; or
S-[2-(1-isobutylcyclohexanecarbonylamino)phenyl]-1-acetyl-piperidine-4-thiocarboxylate, or a pharmaceutically acceptable salt of any of the foregoing.

5. The method of claim 1. wherein the ADCY inhibitor is an ADCY1, ADCY2, ADCY3, ADCY4, ADCY5, ADCY6, ADCY7, ADCY8, ADCY9 or ADCY10 inhibitor.

6. The method of claim 1, wherein the ADCY inhibitor is 9-(tetrahydro-2-furanyl)-adenine); 2′,5′-dideoxyadenosine; 9-cyclopentyladenine; 2′,5′-dideoxyadenosine 3′-diphosphate; 2′,5′-dideoxyadenosine 3′-monophosphate; cis-N-(2-phenylcyclopentyl)azacyclotridece-1-en-2-amine); 2-amino-7-(4-chlorophenyl)-7,8-dihydro-5 (6H)-quinazolinone; 2-amino-7-(4-methoxyphenyl)-7,8-dihydro-5(6H)-quinazolinone; 2-amino-7-phenyl-7,8-dihydro-5(6H)-quinazolinone; 2-amino-7-(2-furanyl)-7,8-dihydro-5(6H)-quinazolinone; 2-amino-7-(2-thienyl)-7,8-dihydro-5(6H)-quinazolinone); 2-amino-7-(4-methoxyphenyl)-7,8-dihydro-5(6H)-quinazolinone; 2-amino-7-phenyl-7,8-dihydro-5(6H)-quinazolinone; 2-amino-7-(2-(uranyl)-7,8-dihydro-5(6H)-quinazolinone; 2-amino-7-(2-thienyl)-7,8-dihydro-5(6H)-quinazolinone); MANT-ATP; MANT-ITP; MANT-GTP; .MANT-XTP; MANT-CTP; MANT-UTP; 2′-MANT-3′d.ATP; 3′-MANT-2′dATP; MANT-ATPγS; MANT-ITPγS; MANT-GTPγS; MANT-UTPγS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANY-ACIP; Bis-MANY-ITP; Bis-MANT-CTP; Bis-MANT-IDP; Bis-MANT-IMP; Bis-Cl-ANT-ATP; Bis-CI-ANT-ITP; Bis-Br-ANT-ATP; Bis-Br-ANT-ITP; Bis-Pr-ANT-ATP; Bis-Pr-ANT-ITP; .Bi s-AcNH-ANT-ATP; Bis-AcNH-ANT-ITP; NKY80; vidarabine; 2′,5′-dd-3′-ATP; AraAde; PJVIC6; NB001; BODIPY-FS; 1,9-dd-FS; 6AIDA-FS; Calmidazolium; Tyrphostin 125; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; 2′,5′-Dideoxyadenosine 3′-triphosphate tetrasodiutn salt; adrenocorticotropic hormone; brain natriuretic peptide (BNP); or pituitary adenylate cyclase-activating polypeptide; or a pharmaceutically acceptable salt of any of the foregoing.

7. The method of claim 1, wherein the cardiovascular disorder is acute coronary syndrome (ACS), atherosclerosis, peripheral vascular disease, dyslipidemia, hyperbetalipoproteinemia, hypoalphalipoproteinemia, hypercholesterollemia, hypertrigtyceridemia, familial-hypercholesterollemia, angina, ischemia, cardiac ischemia, stroke, myocardial infarction, reperfusion injury, angioplastic restenosis, hypertension, cardiovascular disease, coronary heart disease, coronary artery disease, hyperlipidemia, hyperlipidoproteinemia or a vascular complication of diabetes, obesity or endotoxemia.

8. The method of claim 1, wherein the subject is known to have genotype rs11647778/CC, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs8049452/GG, rs8061182/AA, rs2238448/TT, rs12920508/GG, rs2531.9711AA, or rs12599911/GG.

9. The method of claim 1, the subject is known to have genotype rs1967309/AA.

10. The method of claim 1, wherein the subject is known to have genotype rs11647778/CG, rs12595857/AG, rs13337675/AG, rs13337675/GG, rs1967309/AG, rs116478281AG, rs17136707/AG, rs2239310/AG, rs22834971CA, rs2531967/GA, rs3730119/GA, rs4786454/GA, rs8049452/GA, rs8061182/AG, rs2238448/TC, rs12920508/CG, rs2531971/AC, or rs12599911/GT.

11. The method of claim 1, wherein the subject is known to have genotype rs11647778/GG, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs8049452/AA, rs8061182/GG, rs2238448/CC, rs12920508/CC, rs2531971/CC, or rs12599911/TT.

12. A method for reducing risk of a cardiovascular event, comprising administering to a subject in need thereof an effective amount of:
a) a CETP inhibitor; and
b) an ADCY inhibitor.

13. (canceled)

14. The method of claim 12, wherein the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

15. The method of claim 12, wherein the CETP inhibitor is
S-[2-(1-isopentylcyclohexanecarbononylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S-[4,5-dichloro-2-(1-isopentylcyclopentanecarbonylamino)-phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylaminophenyl monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate;
S-[4,5-di chloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate,
S-[4,5-dichloro-2-(1-pentylcyclohexanecarbonylamino)-phenyl]2,2-ditnethylthiopropionate;
S-[4,5-dichloro-2-(1-cyclopropylmethylcyclohexanecarbonylamino)-phenyl]2,2-dimethylthiopropionate;
S-[4,5-dichloro-2-(1-cyclohexylmethylcyclohexanecarbonylamino)phenyl]2,2-dimethylthiopropionate;
S[4,5-dichloro-2-(1-isopropylcyclohexanecarbonylamino)-phenyl]2,2-dimethylthiopropionate;
S-[4,5-dichloro-2-(1-isopentylcycloheptanecarbonylamino)-phenyl]2,2-dimethylthiopropionate,
S-[4,5-dichloro-2-(1-isopentylcyclobutanecarbonylamino)-phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate,
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate;
bis-[4,5-dichloro-2-(1-isopentylcyclohexanecarbonylamino)-phenyl]disulfide;
2-tetrahydrofurylmethyl 2-(1-isopentylcyclohexanecarbonylamino)phenyl disulfide;
N-(2-mercaptophenyl)-1-ethylcyclohexanecarboxamide,
N-(2-mercaptophenyl)-1-propylcyclohexanecarboxamide,
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]ithioacetate;
bis-[2-(1-(2-ethylbutyl)cyclohexanecarbonylaminoiphenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-(2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyyl]-hydroxy-2-methylthiopropionate;
S-[2-[1-(2-ethylbutypcyclohexanecarbonylamino]phenyl]4-chlorophenoxythioacetate;
S-[2-(1-isobutylcyclohexanecarbonylamino)phenyl]-4-chlorophenoxythioacetate; or
S-[2-(1-isobutylcyclohexanecarbonylamino)phenyl]-1-acetyl-piperidine-4-thiocarboxylate, or a pharmaceutically acceptable salt of any of the foregoing.

16. The method of claim 12, wherein the ADCY inhibitor is an ADCY1, ADCY2, ADCY3, ADCY4, ADCY5, ADCY6, ADCY7, ADCY8, ADCY9 or ADCY10 inhibitor.

17. The method of claim 12, wherein the ADCY inhibitor is 9-(tetrahydro-2-furanyl)-adenine); 2′,5′-dideoxyadenosine; 9-cyclopentyladenine; 2′,5′-dideoxyadenosine 3′-diphosphate; 2′,5′-dideoxyadenosine 3′-monophosphate; cis-N-(2-phenylcyclopentyl)azacyclotridece-1-en-2-amine); 2-amino-7-(4-chlorophenyl)-7,8-dihydro-5 (6H)-quinazolinone; 2-amino-7-(4-methoxyphenyl)-7,8-dihydro-5(6H)-quinazolinone; 2-amino-7-phenyl-7,8-dihydro-5(6H)-quinazolinone; 2-amino-7-(2-furanyl)-7,8-dihydro-5(6H)-quinazolinone; 2-amino-7-(2-thienyl)-7,8-dihydro-5(6H)-quinazolinone); 2-amino-7-(4-methoxyphenyl)-7,8-dihydro-5(6H)-quinazolinone; 2-amino-7-phenyl-7,8-dihydro-5(6H)-quinazolinone; 2-amino-7-(2-furanyl)-7,8-dihydro-5(6H)-quinazolinone; 2-amino-7-(2-thienyl)-7,8-dihydro-5(6H)-quinazolinone); MANT-ATP; MANT-ITP; MANT-GTP; MANT-XTP; MANT-CTP; MANT-UTP; 2′-MANT-3′dATP; 3′-MANT-2′dATP; MANT-ATPγS; MANT-UPγS; MANT-GTPγS; MANT-UTPγS; ANT-ATP; Cl-ANT-ATP; Cl-ANT-ITP; Br-ANT-ITP; Pr-ANT-ATP; Pr ANT-M3; AcNH-ANT-ATP; AcNH-ANT-ITP; MANT-AppNHp; MANT-GppNHp; TNP-ATP; TNP-GTP; TNP-CTP; TNP-UTP, Bis-MANT-ATP; Bis-MANT-ITP, Bis-MANT-CTP; Bis-MANT-IDP, Bis-MANT-IMP; Bis-Cl-ANT-ATP; Bis-Cl-ANT-ITP; Bis-Br-ANT-ATP; Bis-Br-ANT-ITP, Bis-Pr-ANT-ATP; Bis-Pr-ANT-ITP; Bis-AcNH-ANT-ATP; Bis-AcNH-ANT-ITP; NKY80; vidarabine; 2′,5′-dd-3′-ATP; AraAde; PMC6; NB001, BODIPY-FS; 1,9-dd-FS, 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine, 2′,5′-Dideoxyadenosine 3′-triphosphate tetrasodium salt; adrenocorticotropic hormone; brain natriuretic peptide (BNP); or pituitary adenylate cyclase-activating polypeptide; or a pharmaceutically acceptable salt of any of the foregoing.

18. The method of claim 12, wherein the cardiovascular event is coronary heart disease, cardiac arrest, myocardial infarction, ischemic stroke, congestive heart failure, sudden cardiac death, cerebral infarction, syncope, transient ischemic attack, angina or coronary revascularization.

19. The method of claim 12, wherein the subject is known to have genotype rs116477781CC, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs8049452/GG, rs8061182/AA, rs2238448/TT, rs12920508/GG, rs2531.971/AA, or rs12599911/GG.

20. The method of claim 12, wherein the subject is known to have genotype rs1967309/AA.

21. The method of claim 12, wherein the subject is known to have genotype
rs11647778/CG, rs12595857/AG, rs13337675/AG, rs13337675/GG, rs1967309/AG, rs11647828/AG, rs17136707/AG, rs2239310AG, rs2283497/CA, rs2531967/GA, rs3730119/GA, rs4786454/GA, rs8049452/GA, rs8061182/AG, rs2238448/TC, rs12920508/CG, rs2531971/AC, or rs12599911/GT.

22. The method of claim 12, wherein the subject is known to have genotype rs11647778/GG, rs12595857/AA, rs13337675/AA, rs1967309/GG, rs111590482/AA, rs11647828/AA, rs12935810/GA, rs12935810AA, rs17136707/A.A, rs22393101AA, rs2283497/CC, rs2531967/GG, rs3730119/GG, rs4786454/GG, rs74702385/GG, rs8049452/AA, rs8061182/GG, rs2238448/CC, rs12920508/CC, rs25319711CC, or rs12599911/TT.

23. A method for treating or preventing a cardiovascular disorder, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor, wherein the ADCY is ADCY1, ADCY2, ADCY3, ADCY4, ADCY5, ADCY6, ADCY7, ADCY8, or ADCY10, and wherein the CETP inhibitor is dalcetrapib, torcetrapib, anacetrapib, evacetrapib, obicetrapib, BMS795311, CP-800,569, 1)LBS-1449, ATH-03, DRL-17822, JNJ-28545595, TM-28614872, BAY 19-4789, BAY 38-1315, or BAY 60-5521, or a pharmaceutically acceptable salt of any of the foregoing.

24. A method for treating or preventing a cardiovascular disorder, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor, wherein the ADCY is ADCY9, and wherein the CETP inhibitor is BMS795311, CP-800,569, JNJ-28545595, TM-28614872, BAY 19-4789, or BAY 38-1315, or a pharmaceutically acceptable salt of any of the foregoing.

25. The method of claim 23 or 24, wherein the subject is known to have reduced expression or activity level of ADCY in the subject's central nervous system compared to a control level.

26. The method of claim 25, wherein the subject is known to have reduced expression or activity level of ADCY in the subject's hypothalamus compared to a control level.

27. The method of claim 23 or 24, wherein the cardiovascular disorder is acute coronary syndrome (ACS), atherosclerosis, peripheral vascular disease, dyslipidemia, hyperbetalipoproteinemia, hypoalphalipoproteinemia, hypercholesterolemia, hypertriglyceridemia, familial-hypercholesterolemia, angina, ischemia, cardiac ischemia, stroke, myocardial infarction, reperfusion injury, angioplastic restenosis, hypertension, cardiovascular disease, coronary heart disease, coronary artery disease, hyperlipidemia, hyperlipidoproteinemia or a vascular complication of diabetes, obesity or endotoxemia.

28. A method for reducing the risk of a cardiovascular event, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor, wherein the ADCY is ADCY1, ADCY2, ADCY3, ADCY4, ADCY5, ADCY6, ADCY7, ADCY8, or ADCY10, and wherein the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

29. A method for reducing the risk of a cardiovascular event, comprising administering to a subject in need thereof an effective amount of a CETP inhibitor, wherein the subject is known to have reduced expression or activity level of ADCY compared to a control level, wherein the reduced expression or activity level of ADCY is indicative that the subject would benefit from administration of the CETP inhibitor, wherein the ADCY is ADCY9, and wherein the CETP inhibitor is BMS795311, CP-800,569, JNJ-28545595, JNJ-28614872, BAY 19-4789, or BAY 38-4315, or a pharmaceutically acceptable salt of any of the foregoing.

30. The method of claim 28 or 29, wherein the subject is known to have reduced expression or activity level of ADCY in the subject's central nervous system compared to a control level.

31. The method of claim 30, wherein the subject is known to have reduced expression or activity level of ADCY in the subject's hypothalamus compared to a control level.

32. The method of claim 28 or 29, wherein the cardiovascular event is coronary heart disease, cardiac arrest, myocardial infarction, ischemic stroke, congestive heart failure, sudden cardiac death, cerebral infarction, syncope, transient ischemic attack, angina or coronary revascularization.

33-36. (canceled)

37. A composition comprising
(a) an effective amount of a CETP inhibitor and an ADCY inhibitor; and
(b) a pharmaceutically acceptable carrier or vehicle.

38. The composition of claim 37, wherein the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

39. The composition of claim 37, wherein the CETP inhibitor is
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl ]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-cabamoylthiobutyrate,
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S-[4,5-dichloro-2-(1-isopentylcyclopentanecarbonylamino)-phenyl]2,2-dimethylthiopropionate;
S-[2-(1-isopentylcyclohexanecarbonylamino)-4-trifluoromethyl phenyl]2,2-dimethylthiopropionate;
O-methyl S-[2-(1-isopentylcyclohexanecarbonylaminophenyl monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate,
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)-phenyl]2,2-dimethylthiopropionate;
S-[4,5-dichloro-2-(1-cyclopropylmethylcyclohexanecarbonylamino)phenyl]12,2-dimethylthiopropionate;
S-[4,5-dichloro-2-(1-cyclohexylmethylcyclohexanecarbonylamino)phenyl]2,2-dimethylthiopropionate;
S-[4,5-dichloro-2-(1-isopropylcyclohexanecarbonylamino)-phenyl]2,2-dimethylthiopropionate;
S-[4,5-dichloro-2-(1-isopentylcycloheptanecarbpnylamino)-phenyl]2,2-dimethylthiopropionate;
S-[4,5-dichloro-2-(1-isopentylcyclocarbonylamino)-phenyl]2,2-dimethylthiopropionate;
S-[2-(-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate;
bis-[4,5-dichloro-2-(1-isopentylcyclohexanecarbonylamino)-phenyl]disulfide;
2-tetrahydrofurylmethyl 2-(1-isopentylcyclohexanecarbonylamino)phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyI)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate,
bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl:]1-acetylpiperidine-4-thiocarboxylate;
S-[2-[1-(2-ethylbutyl)cyclohexanecarbonylamino]phenyl]thioacetate,
S-[2-[1-(2-ethylbutyl)cyclohexanecarbonylamino]phenyl]2,2-dimethylthiopropionate;
S-[2-[1-(2-ethylbutypcyclohexanecarbonylamino]phenyl]methoxythioacetate,
S-[2-[1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; or
S-[2-(1-isobutylcyclohexanecarbonylamino)phenyl]-1-acetyl-piperidine-4-thiocarboxylate, or a pharmaceutically acceptable salt of any of the foregoing.

40. The composition of claim 37, wherein the ADCY inhibitor is an ADCY1, ADCY2, ADCY3, ADCY4, ADCY5, ADCY6, ADCY7, ADCY8, AI)CY9 or ADCY10 inhibitor.

41. The composition of claim 37, wherein the ADCY inhibitor is 9-(tetrahydro-2-furanyl)-adenine); 2′,5′-dideoxyadenosine, 9-cyclopentyladenine; 2′,5′-dideoxyadenosine 3′-diphosphate; 2′,5′-dideoxyadenosine 3′-monophosphate; cis-N-(2-phenylcyclopentyl)azacyclotridece-1-en-2-amine); 2-amino-7-(4-chlorophenyl)-7,8-dihydro-5 (6H)-quinazolinone; 2-amino-7-(4-methoxyphenyl)-7,8-dihydro-5(61I)-quinazolinone, 2-amino-7-phenyl-7,8-dihydro-5(6H)-quinazolinone; 2-amino-7-(2-furanyl)-7,8-dihydro-5(6H)-quinazolinone; 2-amino-7-(2-thienyl)-7,8-dihydro-5(6H)-quinazolinone; 2-amino-7-(4-methoxyphenyl)-7,8-dihydro-5(6H)-quinazolinone; 2-amino-7-phenyl-7,8-dihydro-5(6H)-quinazolinone; 2-amino-7-(2-furanyl)-7,8-dihydro-5(6H)-quinazolinone; 2-amino-7-(2-thienyl)-7,8-dihydro-5(6H)-quinazolinone); MANT-ATP; MANT-ITP; MANT-GTP; MANT-XTP; MANT-CTP; GIANT-UTP; 2′-MANT-3′dATP; 3′-MANT-2′ dATP; MANT-ATPγS; MANT-ITPγS; MANT-GTPγS, MANT-UTPγS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-LMP; Bis-Cl-ANT-ATP; Bis-Cl-ANT-ITP; Bis-Br-ANT-ATP; Bis-Br-ANT-ITP; Bis-Pr-ANT-ATP; Bis-Pr-ANT-ITP; Bis-AcNH-ANT-ATP; Bis-AcNH-ANT-ITP; NKY80; vidarabine; 2′, 5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; 2′,5′-Dideoxyadenosine 3′-triphosphate tetrasodium salt; adrenocorticotropic hormone; brain natriuretic peptide (BNP); or pituitary adenylate cyclase-activating polypeptide; or a pharmaceutically acceptable salt of any of the foregoing.

42-45. (canceled)
